(19) United States
(12) Patent Application Publication
(10) Pub. No.: US 2010/0086546 A1
(43) Pub. Date: Apr. 8, 2010

(54) Toll-LIke Receptor 4 Deficiency and Downstream Effectors Cause Pulmonary Emphysema

(75) Inventors: Patty Lee, Guilford, CT (US); Xuchen Zhang, Orange, CT (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Yale University

(21) Appl. No.: 12/558,308

(22) Filed: Sep. 11, 2009

Related U.S. Application Data
(63) Continuation-in-part of application No. PCT/US08/03418, filed on Mar. 13, 2008.
(60) Provisional application No. 60/906,900, filed on Mar. 13, 2007.

(51) Int. Cl. A61K 39/395; C12Q 1/37; G01N 33/573; C12Q 1/68; A61K 31/7088

(52) U.S. Cl. 424/133.1; 435/23; 435/7.4; 435/6; 424/130.1; 424/141.1; 514/44 A; 514/44 R

(57) ABSTRACT

The present invention provides compositions and methods for the detection, treatment, and prevention of emphysema/COPD. Compositions of the present invention comprise TLR4 activators, Nox3 inhibitors, and Cathepsin E inhibitors useful in the treatment or prevention of emphysema/COPD. Cathepsin E is a downstream effector of TLR4, wherein when cathepsin E is overexpressed in lung of an individual, the individual is at higher risk of developing emphysema/COPD. Cathepsin E is further identified as a biomarker useful in the identification of an individual with, or at-risk of developing emphysema/COPD.

### CROSS REFERENCE TO RELATED APPLICATIONS

This application is a Continuation-in-Part of International Patent Application No. PCT/US2008/003418, filed on Mar. 13, 2008, now published as WO/2008/112307 which claims priority to U.S. Provisional Patent Application No. 60/906,900, filed on Mar. 13, 2007, both of which applications are hereby incorporated by reference in their entirety.

### STATEMENT REGARDING FEDERALLY SPONSORED RESEARCH OR DEVELOPMENT

This invention was made, inpart, using funds obtained from the U.S. Government (National Institutes of Health Grant No. RO1 HL071595, and the U.S. Government therefore has certain rights in this invention.

### BACKGROUND OF THE INVENTION

The lungs are required to process and adapt to the constant exposure to the inhaled environment. The lungs are exposed continuously to oxidants generated either endogenously from phagocytes and other cell types or exogenously from inhaled oxygen as well as pollutants. In addition, intracellular oxidants, such as those derived from the NADPH oxidase (Nox) system, are involved in many cellular signaling pathways. Under normal circumstances lungs can withstand the oxidant challenges imposed by the ambient environment via the presence of well-developed enzymatic and nonenzymatic antioxidant systems. However, when the balance shifts in favor of oxidants, from either an excess of oxidants and/or depletion of its antioxidant responses, oxidative stress occurs.

Chronic obstructive pulmonary disease (COPD) is an umbrella term used to describe progressive, debilitating, or fatal lung disease associated with airflow obstruction. Emphysema and chronic bronchitis, either alone or combined, fall into this category. Emphysema as a major subset of the clinical entity chronic obstructive pulmonary disease (COPD) is defined anatomically as the destruction of the distal lung parenchyma and enlargement of the airspaces.

Pulmonary emphysema is a major cause of morbidity and death worldwide and COPD is the fourth leading cause of death in the U.S., claiming the lives of 122,283 Americans in 2003. It is projected that COPD will be the third leading cause of death in the U.S. by 2020. The National Heart Blood and Lung Institute reports that more than 12 million adults 25 and older are currently diagnosed with COPD with another 12 million harboring as yet undiagnosed COPD. About 1.5 million emergency department visits by and About 726,000 hospitalizations of adults 25 and older were made for COPD in 2000 alone. The total estimated cost of COPD in 2002 was $32.1 billion.

Although cigarette smoking has been recognized as an important factor in the development of COPD, only 10%-20% of heavy smokers develop clinically significant COPD. Furthermore, cigarette smoking is not a prerequisite in the approximately 20% of men who develop COPD. These trends indicate that the development of pulmonary emphysema is affected by other important factors. Several candidate genes have been implicated in determining susceptibility to pulmonary emphysema; however, the precise molecular mechanisms for maintaining the normal oxidant and protease balances remain unclear.

There is an urgent need in the art for new therapeutic targets for the treatment of COPD and emphysema. The present invention fills this need.

### SUMMARY OF THE INVENTION

In one embodiment the present invention comprises a method of identifying an individual at risk of developing emphysema/COPD, the method comprising the steps of: a) measuring the level of Cathepsin E present in a body sample obtained from an individual suspected to be at risk of developing emphysema/COPD; b) comparing the level of Cathepsin E in the body sample to the level of Cathepsin E present in a body sample obtained from an otherwise identical individual not at-risk of developing emphysema/COPD; and, when the level of Cathepsin E is elevated in the mammal suspected to be at risk, compared to the level of Cathepsin E present in a body sample obtained from an otherwise identical individual not at-risk of developing emphysema/COPD, the individual is at-risk for developing emphysema/COPD. In one aspect, the individual is a mammal. In another aspect, the mammal is a human. In still another aspect, the body sample is selected from the group consisting of a tissue, a cell, and a body fluid. In yet another aspect, the body fluid is urine. In one aspect, measuring of Cathepsin E comprises an immunoassay for assessing the level of Cathepsin E in the sample. In another aspect, the immunoassay is selected from the group consisting of Western blot, ELISA, immunoprecipitation, immunohistochemistry, immunofluorescence, radioimmunoassay, dot blotting, and FACS. In still another aspect, measuring Cathepsin E comprises a nucleic acid assay for assessing the level of a nucleic acid encoding said Cathepsin E in the sample. In another aspect, the nucleic assay is selected from the group consisting of a Northern blot, Southern blot, in situ hybridization, a PCR assay, an RT-PCR assay, a probe array, and a gene chip.

Another embodiment of the present invention comprises a method of identifying an individual with emphysema/COPD, the method comprising the steps of: a) measuring the level of Cathepsin E present in a body sample obtained from an individual suspected to have emphysema/COPD; b) comparing the level of Cathepsin E in the body sample to the level of Cathepsin E present in a body sample obtained from an otherwise identical individual who does not have emphysema/COPD; and, when the level of Cathepsin E is elevated in the individual suspected to have emphysema/COPD compared to the level of Cathepsin E present in a body sample obtained from an otherwise identical individual who does not have emphysema/COPD, the individual has emphysema/COPD. In one aspect, the individual is a mammal. In another aspect, the mammal is a human. In still another aspect, the body sample is selected from the group consisting of a tissue, a cell, and a body fluid. In yet another aspect, the body fluid is urine. In one aspect, measuring Cathepsin E comprises an immunoassay for assessing the level of Cathepsin E in the sample. In another aspect, the immunoassay is selected from the group consisting of Western blot, ELISA, immunoprecipitation, immunohistochemistry, immunofluorescence, radioimmunoassay, dot blotting, and FACS. In still another aspect, measuring Cathepsin E comprises a nucleic acid assay for assessing the level of a nucleic acid encoding the Cathepsin E in the sample. In yet another aspect, the nucleic assay is selected from the group consisting of a Northern blot, Southern blot, in situ hybridization, a PCR assay, an RT-PCR assay, a probe array, and a gene chip.

Still another embodiment of the present invention comprises a method of treating a mammal diagnosed with emphysema/COPD, wherein the emphysema/COPD is characterized by lung cell apoptosis, alveolar destruction, increase in lung volume, or loss of lung elasticity, the method comprising administering to the mammal a composition comprising a therapeutically effective amount of at least one Cathepsin E inhibitor wherein the composition attenuates, prevents, or halts the lung cell apoptosis, alveolar destruction, increase in lung volume, or loss of lung elasticity. In one aspect, the Cathepsin E inhibitor comprises an antibody, siRNA, a ribozyme, an antisense, an aptamer, a peptidomimetic, a small molecule, or any combination thereof. In another aspect, the antibody comprises an antibody selected from a polyclonal antibody, a monoclonal antibody, a humanized antibody, a synthetic antibody, a heavy chain antibody, a human antibody, and a biologically active fragment of an antibody. In still another aspect, the mammal is a human.

Still another embodiment of the present invention comprises a method of preventing a mammal at-risk of developing emphysema/COPD from developing emphysema/COPD, wherein the emphysema/COPD is characterized by lung cell apoptosis, alveolar destruction, increase in lung volume, or loss of lung elasticity, the method comprising administering to the mammal a composition comprising a therapeutically effective amount of at least one Cathepsin E inhibitor, wherein the composition prevents said mammal from developing the lung cell apoptosis, alveolar destruction, increase in lung volume, or loss of lung elasticity. In one aspect, the Cathepsin E inhibitor comprises an antibody, siRNA, a ribozyme, an antisense, an aptamer, a peptidomimetic, a small molecule, or any combination thereof. In another aspect, the antibody comprises an antibody selected from a polyclonal antibody, a monoclonal antibody, a humanized antibody, a synthetic antibody, a heavy chain antibody, a human antibody, and a biologically active fragment of an antibody. In still another aspect, the mammal is a human.

Another embodiment of the present invention comprises a method of treating a disease associated with dysregulation of the TLR4 pathway in lung, wherein the dysregulation includes increased Cathepsin E expression, the method comprising administering a therapeutically effective amount of at least one Cathepsin E inhibitor to a mammal wherein the Cathepsin E inhibitor attenuates, prevents, or halts the dysregulation of the TLR4 pathway, thereby reducing the Cathepsin E expression in the lungs of the mammal. In one aspect, the Cathepsin E inhibitor comprises an antibody, siRNA, a ribozyme, an antisense, an aptamer, a peptidomimetic, a small molecule, or any combination thereof. In another aspect, the antibody comprises an antibody selected from a polyclonal antibody, a monoclonal antibody, a humanized antibody, a synthetic antibody, a heavy chain antibody, a human antibody, and a biologically active fragment of an antibody. In another aspect, the mammal is a human.

Still another embodiment of the present invention comprises a method of preventing a disease associated with dysregulation of the TLR4 pathway in lung, wherein the dysregulation includes increased Cathepsin E expression, the method comprising administering a therapeutically effective amount of at least one Cathepsin E inhibitor to a mammal wherein the Cathepsin E inhibitor attenuates, prevents, or halts the dysregulation of the TLR4 pathway, thereby reducing Cathepsin E expression in the lungs of a mammal. In one aspect, the Cathepsin E inhibitor comprises an antibody, siRNA, a ribozyme, an antisense, an aptamer, a peptidomimetic, a small molecule, or any combination thereof. In another aspect, the antibody comprises an antibody selected from a polyclonal antibody, a monoclonal antibody, a humanized antibody, a synthetic antibody, a heavy chain antibody, a human antibody, and a biologically active fragment of an antibody. In still another aspect, the mammal is a human.

Another embodiment of the invention comprises a method of treating a mammal diagnosed with emphysema/COPD, wherein the emphysema/COPD is characterized by lung cell apoptosis, alveolar destruction, increase in lung volume, or loss of lung elasticity, the method comprising administering to the mammal a composition comprising a therapeutically effective amount of at least one Nox 3 inhibitor wherein the composition attenuates, prevents, or halts the lung cell apoptosis, alveolar destruction, increase in lung volume, or loss of lung elasticity. In one aspect, the Nox 3 inhibitor comprises an antibody, siRNA, a ribozyme, an antisense, an aptamer, a peptidomimetic, a small molecule, or any combination thereof. In another aspect, the antibody comprises an antibody selected from a polyclonal antibody, a monoclonal antibody, a humanized antibody, a synthetic antibody, a heavy chain antibody, a human antibody, and a biologically active fragment of an antibody. In still another aspect, the mammal is a human.

Yet another embodiment of the invention comprises a method of preventing a mammal at-risk of developing emphysema/COPD from developing emphysema/COPD, wherein the emphysema/COPD is characterized by lung cell apoptosis, alveolar destruction, increase in lung volume, or loss of lung elasticity, the method comprising administering to the mammal a composition comprising a therapeutically effective amount of at least one Nox 3 inhibitor, wherein the composition prevents the mammal from developing said lung cell apoptosis, alveolar destruction, increase in lung volume, or loss of lung elasticity. In one aspect, the Nox 3 inhibitor comprises an antibody, siRNA, a ribozyme, an antisense, an aptamer, a peptidomimetic, a small molecule, or any combination thereof. In another aspect, the antibody comprises an antibody selected from a polyclonal antibody, a monoclonal antibody, a humanized antibody, a synthetic antibody, a heavy chain antibody, a human antibody, and a biologically active fragment of an antibody. In still another aspect, the mammal is a human.

Still another embodiment of the invention comprises a method of treating a disease associated with dysregulation of the TLR4 pathway in lung, wherein the dysregulation includes increased Nox 3 expression, the method comprising administering a therapeutically effective amount of at least one Nox 3 inhibitor to a mammal wherein the Nox 3 inhibitor attenuates, prevents, or halts the dysregulation of the TLR4 pathway, thereby reducing the Nox 3 expression in the lungs of the mammal. In one aspect, the Nox 3 inhibitor comprises an antibody, siRNA, a ribozyme, an antisense, an aptamer, a peptidomimetic, a small molecule, or any combination thereof. In another aspect, the antibody comprises an antibody selected from a polyclonal antibody, a monoclonal antibody, a humanized antibody, a synthetic antibody, a heavy chain antibody, a human antibody, and a biologically active fragment of an antibody. In still another aspect, the mammal is a human.

Another embodiment of the invention comprises a method of preventing a disease associated with dysregulation of the TLR4 pathway in lung, wherein the dysregulation includes increased Nox 3 expression, the method comprising administering a therapeutically effective amount of at least one Nox 3 inhibitor to a mammal wherein the Nox 3 inhibitor attenuates, prevents, or halts the dysregulation of the TLR4 pathway, thereby reducing Nox 3 expression in the lungs of a mammal. In one aspect, the Nox 3 inhibitor comprises an antibody, siRNA, a ribozyme, an antisense, an aptamer, a peptidomimetic, a small molecule, or any combination thereof. In another aspect, the antibody comprises an antibody selected from a polyclonal antibody, a monoclonal antibody, a humanized antibody, a synthetic antibody, a heavy chain antibody, a human antibody, and a biologically active fragment of an antibody. In still another aspect, the mammal is a human.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIG. 1A through FIG. 1C, is a series of images depicting spontaneous pulmonary emphysema in Tlr4^{-/-} mice. FIG. 1A is a graph depicting lung volumes of WT, Tlr4^{-/-}, and MyD88^{-/-} mice. FIG. 1B is a series of photomicrographs depicting lung histology of WT and Tlr4^{-/-} mice. Original magnification, ×100. FIG. 1C is a graph depicting mean linear chord length in WT, Tlr4^{-/-}, and MyD88^{-/-} mice (n=5-7). Data are mean±SEM. *P<0.05 versus respective WT values.

FIG. 2, comprising FIG. 2A through FIG. 2D, is a series of images depicting decreased elastase inhibitory capacity (EIC) and increased elastin degradation in Tlr4^{-/-} mice. FIG. 2A is a graph depicting EIC detected in BAL of 3-month-old WT and Tlr4^{-/-} mice (n=5-6). FIG. 2B is a graph depicting elastolytic activity assayed in lung lysates of 3-month-old WT and Tlr4^{-/-} mice (n=3). FIG. 2C is a photomicrograph depicting Orcein staining for elastin in the elastic fibers of lung tissue obtained from 3-month-old WT and Tlr4^{-/-} mice. FIG. 2D is a photomicrograph depicting immunohistochemistry using an antibody directed against elastin in the elastic fibers of 3-month-old WT and Tlr4^{-/-} mice. Arrows indicate representative elastin staining in the fibers (brown-black). Original magnification, ×400. Data are mean±SEM. *P<0.05 versus WT.

FIG. 3, comprising FIG. 3A through FIG. 3H, is a series of images depicting decreased antioxidant capacity and increased oxidant burden in Tlr4^{-/-} mice. FIG. 3A is a graph depicting total antioxidant activity was measured in the BAL of 3-month-old WT and Tlr4^{-/-} mice (n=5-6). FIG. 3B is a graph depicting the ratio of reduced GSH to oxidized GSH was detected in BAL of 3-month-old WT and Tlr4^{-/-} mice (n=6). FIG. 3C is a graph depicting the quantitation of flow cytometric results from dihydroethidine staining in total lung cells isolated from 3-month-old WT and Tlr4^{-/-} mice (n=3). MFI, mean fluorescence intensity. FIG. 3D is a graph depicting detection of O₂⁻ production by cytochrome C reduction assays in total lung cells isolated from 3-month-old WT and Tlr4^{-/-} mice (n=3). FIG. 3E is a photomicrograph of lung tissue depicting representative DNA oxidation detection by 8-OH-dG immunohistochemical staining in the lungs of 3-month-old WT and Tlr4^{-/-} mice. Arrow indicates positive staining for 8-OH-dG. Original magnification, ×400. FIG. 3F is a graph depicting quantitation of 8-OH-dG-positive cells expressed as % of total cells in lung sections (n=3). FIG. 3G is a series of representative merged images of lung sections stained with TUNEL and DAPI stain from 3-month-old WT and Tlr4^{-/-} mice. Arrows indicate representative TUNEL-positive cells. Original magnification, ×200 (top panels); ×400 (bottom panels). FIG. 3H is a graph depicting quantitation of TUNEL-positive cells expressed as percent of total cells in lung sections (n=3). Data are mean±SEM. *P<0.05 versus WT.

FIG. 4, comprising FIG. 4A through FIG. 4D, is a series of graphs depicting data that demonstrates antioxidant administration restores the antioxidant activity, EIC, and cell survival in Tlr4^{-/-} mice. FIG. 4A is a graph depicting total antioxidant activity in BAL obtained from wild type, Tlr4^{-/-} mice fed NAC, apocynin, or vehicle-only water and sacrificed at 3 months of age; FIG. 4B is a graph depicting EIC in BAL obtained from wild type, Tlr4^{-/-} mice fed NAC, apocynin, or vehicle-only water and sacrificed at 3 months of age; FIG. 4C is a graph depicting the reduced GSH/oxidized GSH ratio in BAL obtained from wild type, Tlr4^{-/-} mice fed NAC, apocynin, or vehicle-only water and sacrificed at 3 months of age; and FIG. 4D is a graph depicting TUNEL-positive cells (expressed as percent of total cells) in lung sections obtained from wild type or obtained from Tlr4^{-/-} mice fed NAC, apocynin, or vehicle-only water and sacrificed at 3 months of age. *P<0.05 versus WT; **P<0.05 versus Tlr4^{-/-}, n=4-5.

FIG. 5, comprising FIG. 5A through FIG. 5D, is a series of images depicting data that demonstrates that antioxidant administration ameliorates alveolar enlargement in Tlr4−/− mice, whereas adoptive transfer of WT bone marrow cells has no effect. FIG. 5A is a graph depicting lung volume. FIG. 5B is a graph depicting mean linear chord length. n=4-5. FIG. 5C is a photomicrograph depicting representative lung histology stained with H&E. Original magnification, ×100. FIG. 5D is a graph depicting lung volume measured 2 months after adoptive transfer of bone marrow cells from WT mice to WT mice (WT→WT), Tlr4^{-/-} mice to Tlr4^{-/-} mice (Tlr4^{-/-}→Tlr4^{-/-}), WT mice to Tlr4^{-/-} mice (WT→Tlr4^{-/-}), and Tlr4^{-/-} mice to WT mice (Tlr4^{-/-}→WT). Data are mean±SEM. *P<0.05 versus WT or WT→WT; **P<0.05 versus Tlr4^{-/-}.

FIG. 6, comprising FIG. 6A through FIG. 6C, is a series of graphs depicting that TLR4 deficiency leads to increased Nox-mediated elastolytic activity in MLECs. FIG. 6A is a graph depicting elastolytic activity assayed in mouse lung endothelial cells (MLECs) isolated from Tlr4^{-/-} mice treated with DPI (10 μM), NAC (100 μM), or apocynin (10 μM) for 24 hours, (n=3). FIG. 6B is a graph depicting TLR4 mRNA expression in WT MLECs. TLR4 siRNA and nonspecific (NS) siRNA (80 nM) were transfected to WT MLECs, and TLR4 mRNA expression was analyzed by real-time RT-PCR (n=3-5). Ctrl, untransfected control. FIG. 6C is a graph depicting elastolytic activity was assayed in WT MLECs transfected with nonspecific siRNA or TLR4 siRNA (80 nM), and (n=3). Data are mean±SEM. *P<0.05 versus Tlr4^{-/-} (FIG. 6A), control and nonspecific siRNA (FIG. 6B), and WT and WT transfected with nonspecific siRNA (FIG. 6C).

FIG. 7, comprising FIG. 7A through FIG. 7F, is a series of images depicting that TLR4 deficiency leads to increased Nox3 expression in lung and lung endothelial cells. FIG. 7A is a graph depicting Nox3 mRNA expression in 3-month-old WT and Tlr4^{-/-} mouse lungs as detected by real-time RT-PCR (n=3). FIG. 7B is an image of a gel depicting Nox3 protein expression in 3-month-old WT and Tlr4^{-/-} mouse lungs (n=3). FIG. 7C is a graph depicting Nox3 mRNA expression in MLECs isolated from WT and Tlr4^{-/-} mice as detected by real-time RT-PCR (n=3). FIG. 7D is a graph depicting Nox3 mRNA expression in WT MLECs transfected with nonspecific siRNA or TLR4 siRNA (80 nM) as detected by real-time RT-PCR compared with untransfected controls (n=3). FIG. 7E is a graph depicting Nox3 mRNA expression in WT MLECs transfected with nonspecific siRNA or Nox3 siRNA (80 nM) as detected by real time RT-PCR compared with untransfected controls (n=3). FIG. 7F is a graph depicting elastolytic activity was detected in Tlr4^{-/-} MLECs transfected with Nox3 siRNA or nonspecific siRNA and WT MLECs transfected with TLR4 siRNA or nonspecific siRNA (n=3). Data are shown as mean±SEM. *P<0.05 versus WT (FIG. 7A and FIG. 7C), untransfected controls (FIG. 7D and FIG. 7E), and Tlr4^{-/-} (FIG. 7F).

FIG. 8 is a chart depicting the construct used to generate inducible, human Cathepsin E transgenic mice (hereafter referred to as iTg+, which when fed doxycycline-containing water will induce the human Cathepsin E transgene and result in increased levels of Cathepsin E protein).

FIG. 9, comprising FIG. 9A and FIG. 9B, is a series of images depicting the results of genotyping trangensic animals as well as the detection of inducible Cathepsin E in transgenic animals. FIG. 9A depicts the results of genotyping wildtype (Tg−) and inducible, Cathepsin E transgenic mice (iTg+). FIG. 9B depicts detection of inducible and increased levels of human Cathepsin E protein in iTg+ mice and Tg− mice using Western analysis. β-Actin levels were used to control for protein loading.

FIG. 10 is an image depicting organ specificity of Cathepsin E (Cathepsin E) protein expression in iTg+ mice compared to Tg− mice. Detection of inducible and increased levels of human Cathepsin E protein in iTg+ mice as compared to Tg− mice was done using Western analysis. β-Actin levels were used to control for protein loading.

FIG. 11 is a graph depicting increased levels of Cathepsin E protein levels in the broncho-alveolar lavage fluid (BAL) of iTg+ mice compared to Tg− mice.

FIG. 12 is a graph depicting detectable Cathepsin E (Cathepsin E) in urine of wild type and transgenic mice. Transgenic mice that expressed constitutively active Cathepsin E protein in a lung-targeted manner (Cathepsin E Tg+) show increased levels of Cathepsin E detectable in urine compared to wildtype (WT) mice.

FIG. 13, comprising FIG. 13A and FIG. 13B, is a pair of photomicrographs depicting immunohistochemical staining of lung sections from wild type (Cathepsin E Tg−) and Cathepsin E Tg+ mice. Staining of Cathepsin E protein in the lung epithelial cells is seen in lung sections is obtained from Cathepsin E Tg+ mice (arrow). The image is 20× original magnification.

FIG. 14 is a graph depicting the effect of targetted Cathepsin E expression in lungs of transgenic animals over time. The graph compares lung volumes measured in Cathepsin E Tg− and Cathpsin E Tg+ mice at 1, 2, and 3 months.

FIG. 15, comprising FIG. 15A and FIG. 15B, is a photomicrograph depicting Orcein staining for elastin in the elastic fibers of lung tissue obtained from Cathepsin E Tg⁻ and Cathepsin E Tg⁺ mice. Arrows indicate representative elastin staining in the fibers (brown-black). Original magnification, ×40.

FIG. 16, comprising FIG. 16A and FIG. 16B, is a pair of photomicrographs depicting immunohistochemical staining of lung sections for TUNEL staining in order to detect apoptosis in lungs from wild type (Cathepsin E Tg−) and Cathepsin E Tg+ mice. FIG. 16A depicts lung tissue obtained from wild type (Cathepsin E Tg−). FIG. 16B depicts lung tissue obtained from mice that are iTg+, in which Cathepsin E expression is induced. The lungs from iTg+ mice show increased numbers of TUNEL-positive/apoptotic cells (arrows) compared to Tg− mice.

FIG. 17, comprising FIG. 17A and FIG. 17B, is a series of images depicting increased levels of Bax, a pro-apoptotic protein, in the lungs of iTg+ mice. FIG. 17A is an image depicting increased levels of Bax protein detected in the lungs of iTG+ mice as compared to wild type (Cathepsin E Tg−) using Western analysis. β-Actin levels were used to control for protein loading. FIG. 17B is a graph depicting Bax protein concentration detected in lung obtained from iTG+ mice as compared to wild type (Cathepsin E Tg−). The y-axis represents fold induction of protein compared to wild-type mice.

FIG. 18, comprising FIG. 18A and FIG. 18B, is a series of images depicting increased levels of Bcl/xL, an anti-apoptotic protein, in the lungs of iTg+ mice. FIG. 18A is an image depicting increased levels of Bax protein detected in the lungs of wild type (Cathepsin E Tg−) as compared to iTG+ mice using Western analysis. β-Actin levels were used to control for protein loading. FIG. 18B is a graph depicting Bcl/xL protein concentration detected in lung obtained from iTG+ mice as compared to wild type (Cathepsin E Tg−). The y-axis represents fold induction of protein compared to wild-type mice.

FIG. 19 is a graph depicting the ability to silence Cathepsin E mRNA in lung endothelial cells using Lentiviral Cathepsin E siRNA.

FIG. 20, comprising FIG. 20A and FIG. 20B, is a series of graphs depicting the ability of Cathepsin E siRNA to decrease elastolytic activity in TLR4−/− mice. FIG. 20A is a graph depicts Cathepsin E siRNA dose response. Cat E mRNA expression in TLR4−/− Ec transfected with nonspecific (NS) siRNA (50 mM) or Cat E siRNA (25 nM to 50 nM) was detected by qRT-PCR compared with untransfected controls (CTRL) (n=3). FIG. 20B is a graph depicting the effect of Cathepsin E siRNA on elastolytic activity. Elastolytic activity was detected in TLR4−/− Ec transfected with Nox3 siRNA, Cathepsin E siRNA or NS siRNA and WT Ec transfected with TLR4 siRNA or NS siRNA (n=3). *P<0.05 vs CTRL (FIG. 20A) and vs TLR4−/− (FIG. 20B).

FIG. 21, comprising FIG. 21A and FIG. 21B, is a series of images depicting the ability of Cathepsin E siRNA to decrease elastolytic activity in TLR4−/− mice. FIG. 21A is an image of a gel depicting Cathepsin E protein levels detected by Western Blot analysis in TLR4−/− mice administered intranasal Cathepsin E siRNA or nonspecific (NS) siRNA (2 mg/kg body weight). FIG. 21B is a graph depicting elastolytic activity detected in TLR4−/− mouse BAL after Cathepsin E siRNA or NS siRNA administration (n=3). Data shown as mean±SEM. *P<0.05 vs wild type and Cathepsin E siRNA.

FIG. 22, comprising FIG. 22A and FIG. 22B, is a series of graphs depicting Cathepsin E and Carboxypeptidase activity in Cathepsin E Tg mice. FIG. 22A is a graph depicting Cathpesin E activity in lung tissues. Cat E activity in lung tissues was determined by its ability to cleave a fluorogenic Cathepsin E substrate, MOCAc-Gly-Ser-Pro-Ala-Phe-Leu-Ala-Lys (dnp)-D-Arg-NH2 at pH 3.5 and 7.4. Activity was expressed as ng Cat E/mg protein in the lysates, according to a standard curve. FIG. 22B is a graph depicting Carboxypeptidase A activity in lung tissues. Carboxypeptidase A activity in lung tissue was determined by its ability to cleave a fluorescent carboxypeptidase A substrate, (7-Methoxycoumarin-4-yl) acetyl-Arg-Pro-Pro-Gly-Phe-Ser-Ala-Phe-Lys (2,4-Dinitrophenyl)-OH and expressed as relative fluorescence unit (RFU). Data are shown as mean±SEM. *P<0.05 vs Cat E Tg− (n=4).

FIG. 23, comprising FIG. 23A through FIG. 23D, is a series of images depicting the positive association of Cathepsin E expression in human lung with cigarette smoking. FIG. 23A is a lung section obtained from a non-smoking human and exhibits normal Cathepsin E expression levels as well as normal histology. FIG. 23B and FIG. 23C both depict lung sections obtained from smokers. FIG. 23D is a graph depicting the relationship between Cathepsin E expression (Cathepsin E score) and whether an individual is a non-smoker, a current smoker, or an ex-smoker.

FIG. 24 is a graph depicting the association of Cathepsin E with emphysema and/or COPD in humans.

FIG. 25, comprising FIG. 25A through FIG. 25D, is a series of graphs depicting the effect of TLR deficiency on Nox3 expression in endothelial cells (EC) and lung. FIG. 25A is a graph depicting Nox3 mRNA expression in 3 month old WT and TLR4−/− mouse lungs as detected by qRT-PCR (n=3). FIG. 25B is an image of a gel depicting Nox3 protein expression in 3 month old WT and TLR4−/− mouse lungs (n=3). FIG. 25C is a graph depicting Nox3 mRNA expression in EC isolated from WT and TLR4−/− mice as detected by qRT-PCR (n=3). FIG. 25D is a graph depicting Nox3 mRNA expression in WT EC transfected with nonspecific (NS) siRNA or TLR4 siRNA (80 nM) as detected by qRT-PCR compared with untransfected controls (n=3). Data are shown as mean±SEM. *P<0.05 vs WT (FIG. 22A and FIG. 22 and C) and vs untransfected controls (FIG. 22D).

FIG. 26, comprising FIG. 26A and FIG. 26B, is a series of graphs depicting the effect of increased Nox3 expression on elastolytic activity in EC. FIG. 26A is a graph depicting Nox3 mRNA expression in WT Ec transfected with nonspecific (NS) siRNA or Nox3 siRNA (80 nM) as detected by qRT-PCR and compared to untransfected controls (CTRL) (n=3). FIG. 26B is a graph depicting elastolytic activity detected in TLR4−/− Ec transfected with Nox3 siRNA or NS siRNA and WT Ec transfected with TLR4 siRNA or NS siRNA (n=3). Data are shown as mean SEM. *P<0.05 vs CTRL (FIG. 26A) and vs TLR4−/− (FIG. 26B).

FIG. 27, comprising FIG. 27A through FIG. 27C, is a series of images depicting Nox3 localization in TLR4−/− mice. FIG. 27A is a photomicrograph depicting in situ hybridization of Nox3 mRNA in 3 month old TLR4−/− mouse lungs. The single arrow, double arrow and arrow head indicate the dark blue positively stained airway epithelium, endothelium and type II alveolar epithelium, respectively. A sense Nox3 probe was used as a negative control. FIG. 27B is a series of images depicting immunofluorescence of Nox3 in TLR4−/− lung. Antibodies targeting Nox3 (left panels) and cell-specific markers (middle panels) were used to identify Nox3 expression in airway epithelium (CC10), type II alveolar epithelium (Sp-C), and endothelium (CD31). The color of fluorescence detected varied depending on the secondary antibody used. Merged images are shown in the right panels. Arrows indicate double-positive cells. Negative controls were performed without the primary antibody (not shown). FIG. 27C is a series of graphs depicting Nox3 mRNA expression in alveolar epithelial cells and BAL macrophages isolated from WT and TLR4−/− mice as detected by qRT-PCR (n=3). Data are shown as mean±SEM. *P<0.05 vs WT.

FIG. 28, comprising FIG. 28A and FIG. 28B, is a series of images depicting the lentiviral expression system and subsequent expression of lentiviral GFP after intranasal administration in mice. FIG. 28A is a diagram of the lentiviral construct for green fluorescent protein (GFP) or Nox3 overexpression. FIG. 28B is a series of images depicting GFP expression in airway epithelium of mice given intranasal lentiviral GFP. Antibodies targeting GFP (left panels) and cell-specific markers (middle panels) were used to identify GFP expression in airway epithelium (CC10), type II alveolar epithelium (Sp-C), and endothelium (CD31). The color of fluorescence detected (green or red) varied depending on the secondary antibody used. Merged images are shown in the right panels. Arrows indicate doublepositive cells. Negative controls were performed without the primary antibody (not shown).

FIG. 29, comprising FIG. 29A through FIG. 29D, is a series of images depicting Lenti-Nox3 overexpression in lung. FIG. 29A1 is an image of a gel depicting increased Nox3 mRNA with lentiviral Nox 3 (lenti-Nox3). Nox3 mRNA expression was detected in lungs from naïve, lenti-GFP and lenti-Nox3-treated mice by RT-PCR. FIG. 29A2 is a graph depicting Nox3 mRNA expression was detected in lungs from naïve, lenti-GFP and lenti-Nox3-treated mice by qRT-PCR (n=4). FIG. 29B is a graph depicting lung lipid peroxidation detected at 3 months of age (n=4). FIG. 29C is a graph depicting lung volume assessed at 3 months of age (n=4). Data are shown as mean±SEM. *P<0.05, vs lenti-GFP.

FIG. 30, comprising FIG. 30A through FIG. 30C, is a series of graphs depicting distinct TLR4 pathways regulating Cathepsin E and Nox3. FIG. 30A is a graph depicting Cathepsin E and Nox3 mRNA detected by qRT-PCR in WT, Trif−/− and MyD88−/− mice, (n=5). FIG. 30B is a graph depicting total antioxidant activity measured in BAL of the same mice as in FIG. 30A (n=3-5). FIG. 30C is a graph depicting lung volumes of 1 month (1 m), 3 month (3 m), and 6 month (6 m) old WT or Trif−/− mice. Data are shown as mean±SEM. *P<0.05, vs WT.

FIG. 31 is a graph depicting elevated serum levels of cathepsin E in human subjects with COPD as compared to non-COPD subjects.

### DETAILED DESCRIPTION OF THE INVENTION

It is demonstrated herein that TLR4 expression in the lung as well as TLR4 downstream effectors are required to maintain lung structural integrity. TLR4 regulates reactive oxidant generation by suppressing NADPH oxidase (Nox3) in lung endothelial cells and thereby prevents lung cell death and tissue destruction. Cathepsin E is regulated by TLR4 and its increased expression in lung tissue is correlated with pathophysiological changes consistent with emphysema including increased lung cell apoptosis, increased lung volumes, and decreased lung elasticity. Cathepsin E is a secreted protein. Its elevated expression is detectable in several accessible biological compartments, making it not only an attractive therapeutic target in the treatment of emphysema, but a useful screening marker for emphysema.

Thus, the present invention includes compositions and methods for diagnosis and treatment of emphysema/COPD capitalizing on the role of Cathepsin E in this disease.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

The phrase “activator,” as used herein, means to increase a molecule, a reaction, an interaction, a gene, an mRNA, and/or a protein's expression, stability, function or activity by a measurable amount. Activators are compounds that, e.g., bind to, partially or totally stimulate, increase, promote, increase activation, activate, sensitize, or up-regulate a protein, a gene, and an mRNA stability, expression, function and activity.

The term “BAL,” as used herein, refers to bronchoalveolar lavage fluid.

The term “chronic obstructive pulmonary disease,” or COPD, is used herein to refer to two lung diseases, chronic bronchitis and emphysema, that are characterized by obstruction to airflow that interferes with normal breathing. Both of these conditions frequently co-exist.

The term “emphysema” is a major subset of the clinical entity known as COPD and is characterized by specific pathological changes in lung tissue over time. One hallmark of emphysema is the gradual, progressive, and irreversible destruction of the distal lung parenchyma leading to the destruction alveoli. Alveolar destruction leads to enlarged airspaces in the lung and consequently a reduced ability to transfer oxygen to the bloodstream. Emphysema is also characterized by a loss of elasticity in the lung making it difficult to maintain open airways. Both of these changes produce the clinical sequalae of emphysema comprising shortness of breath and difficulty exhaling, respectively.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

As used herein, the term “heavy chain antibody” or “heavy chain antibodies” comprises immunoglobulin molecules derived from camelid species, either by immunization with an antigen and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

By the term “applicator,” as the term is used herein, is meant any device including, but not limited to, a hypodermic syringe, a pipette, and the like, for administering the compounds and compositions of the invention.

As used herein, “aptamer” refers to a small molecule that can bind specifically to another molecule. Aptamers are typically either polynucleotide- or peptide-based molecules. A polynucleotidal aptamer is a DNA or RNA molecule, usually comprising several strands of nucleic acids, that adopt highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

The phrase “body sample” as used herein, is intended any sample comprising a cell, a tissue, or a bodily fluid in which expression of the biomarker, Cathepsin E, can be detected. Examples of such body samples include but are not limited to blood, lymph, urine, gynecological fluids, biopsies, amniotic fluid and smears. Samples that are liquid in nature are referred to herein as “bodily fluids.”Body samples may be obtained from a patient by a variety of techniques including, for example, by scraping or swabbing an area or by using a needle to aspirate bodily fluids. Methods for collecting various body samples are well known in the art.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, preferably at least about 60% and more preferably at least about 80% of corresponding positions in each of the molecules are occupied by nucleotides which normally base pair with each other (e.g., A:T and G:C nucleotide pairs).

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

Signal transduction is any process by which a cell converts one signal or stimulus into another, most often involving ordered sequences of biochemical reactions carried out within the cell. The number of proteins and molecules participating in these events increases as the process emanates from the initial stimulus resulting in a “signal cascade.” The phrase “downstream effector”, as used herein, refers to a protein or molecule acted upon during a signaling cascade, which in term acts upon another protein or molecule. The term “downstream” indicates the direction of the signaling cascade.

A disease or disorder is “alleviated” if the severity of a symptom of the disease, or disorder, the frequency with which such a symptom is experienced by a patient, or both, are reduced.

The terms “effective amount” and “pharmaceutically effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

The term “dysregulation” as used herein describes an over- or under-expression of a component of the TLR4 pathway essential for maintaining the structural integrity of the lung in an individual with emphysema/COPD as compared to a normal, not-at-risk individual.

A “putative at-risk individual” is a mammal, preferably a human, who is thought to be at risk of developing emphysema/COPD.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly five amino acids and/or sugars in size. One skilled in the art understands that generally the overall three-dimensional structure, rather than the specific linear sequence of the molecule, is the main criterion of antigenic specificity and therefore distinguishes one epitope from another.

As used herein, the term “exogenous” refers to any material introduced from or produced outside an organism, cell, tissue or system.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

By “expression cassette” is meant a nucleic acid molecule comprising a coding sequence operably linked to promoter/regulatory sequences necessary for transcription and, optionally, translation of the coding sequence.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a n inducible manner.

An “inducible” promoter is a nucleotide sequence which, when operably linked with a polynucleotide which encodes or specifies a gene product, causes the gene product to be produced substantially only when an inducer which corresponds to the promoter is present.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

As used herein, a “peptidomimetic” is a compound containing non-peptidic structural elements that is capable of mimicking the biological action of a parent peptide. A peptidomimetic may or may not comprise peptide bonds.

A “polynucleotide” means a single strand or parallel and anti-parallel strands of a nucleic acid. Thus, a polynucleotide may be either a single-stranded or a double-stranded nucleic acid. In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

The term “recombinant DNA” as used herein is defined as DNA produced by joining pieces of DNA from different sources.

The term “recombinant polypeptide” as used herein is defined as a polypeptide produced by using recombinant DNA methods.

“Ribozymes” as used herein are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules. Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053).

By the term “specifically binds,” as used herein, is meant a molecule, such as an antibody, which recognizes and binds to another molecule or feature, but does not substantially recognize or bind other molecules or features in a sample. As used herein, the term “transdominant negative mutant gene” refers to a gene encoding a protein product that prevents other copies of the same gene or gene product, which have not been mutated (i.e., which have the wild-type sequence) from functioning properly (e.g., by inhibiting wild type protein function). The product of a transdominant negative mutant gene is referred to herein as “dominant negative” or “DN” (e.g., a dominant negative protein, or a DN protein).

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

The phrase “Cathepsin E inhibitor,” as used herein, refers to a composition or compound that inhibits Cathepsin E activity, either directly or indirectly, using any method known to the skilled artisan. A Cathepsin E inhibitor may be any type of compound, including but not limited to, a polypeptide, a nucleic acid, an aptamer, a peptidometic, and a small molecule.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

It is understood that any and all whole or partial integers between any ranges set forth herein are included herein.

### DESCRIPTION

The invention is based in part on the discovery that disruption of the Toll-like receptor 4 (TLR4) pathway contributes to an individual's susceptibility to emphysema. As shown herein, the TLR4 pathway in lung comprises TLR4 regulation of oxidant stress via downstream effector regulation including regulation of both Nox3 and Cathepsin E activity or expression.

TLR4 expression is required to maintain normal lung structural integrity by modulating oxidant generation in the lung in response to both injury-induced as well as ambient oxidant stressors. Dysregulation of the TLR4 pathway, such as TLR4 deficiency in lung endothelial cells in Tlr4^{-/-} mice, lung-targeted Cathepsin E overexpression, or lung-targeted Nox3 overexpression, leads to pathophysiological changes in lung consistent with emphysema including: elastin degradation, lung cell apoptosis, alveolar destruction, increased lung volume, and decreased lung elasticity in the absence of overt lung inflammation. It is further demonstrated that Cathepsin E and Nox3 are useful biomarkers associated with smoking and emphysema/COPD in humans.

Accordingly, the present invention provides compositions and methods useful in identifying an individual with emphysema/COPD, or an individual at risk of developing emphysema/COPD. In one embodiment, the method comprises measuring the amount of Cathepsin E present in a body sample obtained from an individual diagnosed with emphysema/COPD, an individual at risk of developing emphysema/COPD, an individual suspected to have emphysema/COPD, or an individual thought to have emphysema/COPD. When the level of the Cathepsin E measured in the body sample is elevated compared to the level of Cathepsin E in an otherwise identical individual who does not have, nor is at risk for, emphysema/COPD, that individual has emphysema/COPD or is at risk of developing emphysema/COPD. In another embodiment, the method comprises measuring the amount of Nox3 present in a body sample obtained from an individual diagnosed with emphysema/COPD, an individual at risk of developing emphysema/COPD, an individual suspected to have emphysema/COPD, or an individual thought to have emphysema/COPD. When the level of the Nox3 measured in the body sample is elevated compared to the level of Nox3 in an otherwise identical individual who does not have, nor is at risk for, emphysema/COPD, that individual has emphysema/COPD or is at risk of developing emphysema/COPD. A body sample is any biological sample obtained from an individual including but not limited to urine, serum, sputum, and bronchoalveolar lavage fluid (BAL).

In one aspect of the invention, the individual is a mammal. In a preferred aspect of the invention, the mammal is a human.

In one embodiment of the invention, measuring Cathepsin E or Nox3 comprises an immunoassay for assessing the level of Cathepsin E of Nox3 protein in a body sample, wherein the immunoassay is selected from the group consisting of Western blot, ELISA, immunoprecipitation, immunohistochemistry, immunofluorescence, radioimmunoassay, dot blotting, and FACS.

In another embodiment of the invention, measuring of Cathepsin E or Nox3 comprises a nucleic acid assay for assessing the level of a nucleic acid encoding Cathepsin E or Nox3 in a body sample, wherein the nucleic assay is selected from the group consisting of a Northern blot, Southern blot, in situ hybridization, a PCR assay, an RT-PCR assay, a probe array, a gene chip, and a microarray.

The method is useful in identifying an individual with emphysema/COPD or at-risk of developing emphysema/COPD comprises detecting or measuring Cathepsin E in a body sample obtained from an individual diagnosed with emphysema/COPD, or a putative at-risk individual, then comparing the levels of Cathepsin E present in the test sample to Cathepsin E levels detected or measured in a sample obtained from one or more otherwise identical, normal, not-at-risk individuals. In some instances, the level of Cathepsin E expression is compared with an average value obtained from more than one not-at-risk individuals. In other instances, the level of Cathepsin E expression is compared with Cathepsin E assessed in a sample obtained from one normal, not-at-risk sample. In yet another instance, the level of Cathepsin E expression in the putative at-risk individual is compared with the level of Cathepsin E expression in a sample obtained from the same individual at a different time.

The method is also useful in identifying an individual with emphysema/COPD or at-risk of developing emphysema/COPD comprises detecting or measuring Nox3 in a body sample obtained from an individual diagnosed with emphysema/COPD, or a putative at-risk individual, then comparing the levels of Nox3 present in the test sample to Nox3 levels detected or measured in a sample obtained from one or more otherwise identical, normal, not-at-risk individuals. In some instances, the level of Nox3 expression is compared with an average value obtained from more than one not-at-risk individuals. In other instances, the level of Nox3 expression is compared with Nox3 assessed in a sample obtained from one normal, not-at-risk sample. In yet another instance, the level of Nox3 expression in the putative at-risk individual is compared with the level of Nox3 expression in a sample obtained from the same individual at a different time.

The invention further provides compositions and methods useful in treating an individual diagnosed with emphysema/COPD. Treating an individual diagnosed with emphysema/COPD encompasses a method of inhibiting the progression of emphysema/COPD in an individual diagnosed with emphysema/COPD. By “inhibiting the progression of emphysema/COPD” is intended to mean that the progressive histological and morphometric changes associated with the clinical sequalae of emphysema/COPD, including lung cell death, elastolysis, alveolar destruction and loss of lung elasticity, are halted, prevented, or attenuated. It will be appreciated that the method of the present invention may also be practiced in an individual at risk of developing COPD whereby an individual identified as being at risk of developing emphysema/COPD may be prevented from developing or experiencing lung cell death, elastolysis, alveolar destruction and loss of lung elasticity, that would subsequently lead to a clinical manifestation of emphysema/COPD.

The methods of the invention comprise administering a therapeutically effective amount of a Cathepsin E inhibitor to an individual with emphysema/COPD or an individual at risk of developing emphysema/COPD where the inhibitor reduces or prevents, halts, or attenuates lung cell death, elastolytic activity, alveolar destruction, or loss of lung elasticity.

The methods of the invention comprise administering a therapeutically effective amount of a Nox3 inhibitor to an individual with emphysema/COPD or an individual at risk of developing emphysema/COPD where the inhibitor reduces or prevents, halts, or attenuates lung cell death, elastolytic activity, alveolar destruction, or loss of lung elasticity.

The methods of the invention further comprise administering a therapeutically effective amount of a TLR4 activator to an individual with emphysema/COPD or an individual at risk of developing emphysema/COPD, wherein the TLR4 activator reduces, prevents, or attenuates lung cell death, elastolytic activity, alveolar destruction and loss of lung elasticity.

The methods of the invention further comprise administering a therapeutically effective amount of a TLR1 activator to an individual with emphysema/COPD or an individual at risk of developing emphysema/COPD, wherein the TLR4 activator reduces, prevents, or attenuates lung cell death, elastolytic activity, alveolar destruction and loss of lung elasticity.

The methods of the invention further comprise administering a therapeutically effective amount of a TLR2 inhibitor to an individual with emphysema/COPD or an individual at risk of developing emphysema/COPD, wherein the TLR4 activator reduces, prevents, or attenuates lung cell death, elastolytic activity, alveolar destruction and loss of lung elasticity.

The methods of the present invention may be practiced on any individual diagnosed with, or at risk of developing emphysema/COPD. Preferably the individual is a human. An individual may have emphysema/COPD, or be at risk of developing emphysema/COPD because of a history of smoking, exposure to environmental pollutants, toxins, infectious agents, or other compounds that may induce lung cell death, elastolysis, alveoli destruction, or loss of lung elasticity.

Inhibiting Cathepsin E activity can be accomplished using any method known to the skilled artisan. Examples of methods to inhibit Cathepsin E activity include, but are not limited to decreasing expression of an endogenous Cathepsin E gene, decreasing expression of Cathepsin E mRNA, and inhibiting activity of Cathepsin E protein. A Cathepsin E inhibitor may therefore be a compound or composition that decreases expression of a Cathepsin E gene, a compound or composition that decreases Cathepsin E mRNA half-life, stability and/or expression, or a compound or composition that inhibits Cathepsin E protein function. A Cathepsin E inhibitor may be any type of compound, including but not limited to, an antibody, a polypeptide, a nucleic acid, an aptamer, a peptidometic, and a small molecule, or combinations thereof.

Cathepsin E inhibition may be accomplished either directly or indirectly. For example, Cathepsin E may be directly inhibited by compounds or compositions that directly interact with Cathepsin E protein, such as antibodies or proteinase inhibitors. Alternatively, Cathepsin E may be inhibited indirectly by compounds or compositions that inhibit Cathepsin E downstream effectors, or upstream regulators which up-regulate Cathepsin E expression.

Decreasing expression of an endogenous Cathepsin E gene includes providing a specific inhibitor of Cathepsin E gene expression. Decreasing expression of Cathepsin E mRNA or Cathepsin E protein includes decreasing the half-life or stability of Cathepsin E mRNA or decreasing expression of Cathepsin E mRNA. Methods of decreasing expression of Cathepsin E include, but are not limited to, methods that use an siRNA, a microRNA, an antibody, a proteinase, an antisense nucleic acid, a ribozyme, an expression vector encoding a transdominant negative mutant, a peptide, a small molecule, other specific inhibitors of Cathepsin E gene, mRNA, and protein expression, and combinations thereof.

Inhibiting Nox3 expression or its activity may be accomplished by any means known in the art or as described herein in methods that are substantially the same as those described herein for inhibition of Cathpesin E activity or expression. Similarly, inhibiting TLR2 expression or activity may be accomplished by any means known in the art or as described herein in methods that are substantially the same as those described herein for inhibition of Cathpesin E activity or expression.

Enhancing or increasing TLR4 expression or activity can be accomplished using any method known to the skilled artisan. Examples of methods to enhance or increase TLR4 expression include, but are not limited to increasing expression of an endogenous TLR4 gene, increasing expression of TLR4 mRNA, and increasing expression of TLR4 protein. An agent, composition or compound that enhances or increases TLR4 expression or activity may be a compound or composition that increases expression of a TLR4 gene, a compound or composition that increases TLR4 mRNA half-life, stability and/or expression, or a compound or composition that enhances TLR4 protein function. An agent, composition or compound that enhances or increases TLR4 expression or activity may be any type of compound, including but not limited to, a polypeptide, a nucleic acid, an aptamer, a peptidometic, and a small molecule, or combinations thereof.

Activation of TLR1 may be accomplished by any means known in the art or as described herein in methods that are substantially the same as those described herein for activating TLR4 activity or expression.

The present invention should in no way be construed to be limited to the inhibitors or activators described herein, but rather should be construed to encompass any activator or inhibitor of the TLR system in lung, both known and unknown, that promotes lung structural integrity or prevents, attenuates, or halts the development of pathophysological changes in lung associated with emphysema/COPD.

### I. Compositions

A. Cathepsin E and Nox3 Inhibitors

1. Antibodies

In one embodiment of the invention, the Cathepsin E or Nox3 inhibitor is an antibody. It will be appreciated by one skilled in the art that an antibody comprises any immunoglobulin molecule, whether derived from natural sources or from recombinant sources, which is able to specifically bind to an epitope present on a target molecule. In the present invention, the target molecule may be Cathepsin E, Nox3, or fragments thereof. In one aspect of the invention, Cathepsin E is directly inhibited by an antibody that specifically binds to an epitope on Cathepsin E. In another aspect of the invention, the effects of Cathepsin E are blocked by an antibody that specifically binds to an epitope on a downstream effector such as extracellular matrix (ECM) proteins, proteases, anti-proteases, transcription factors, fibrogenetic cytokines, or apoptosis regulators. In still another aspect of the invention, the effects of Cathepsin E are blocked by an antibody that binds to an epitope of an upstream regulator of Cathepsin E.

In one aspect of the invention, Nox3 is directly inhibited by an antibody that specifically binds to an epitope on Nox3. In another aspect of the invention, the effects of Nox3 are blocked by an antibody that specifically binds to an epitope on a downstream effector such as extracellular matrix (ECM) proteins, proteases, anti-proteases, transcription factors, fibrogenetic cytokines, or apoptosis regulators. In still another aspect of the invention, the effects of Nox3 are blocked by an antibody that binds to an epitope of an upstream regulator of Nox3.

When the Cathepsin E inhibitor used in the compositions and methods of the invention is a polyclonal antibody (IgG), the antibody is generated by inoculating a suitable animal with a peptide comprising full length Cathepsin E protein (SEQ ID NO.: 1), or a fragment thereof, an upstream regulator, or fragments thereof. These polypeptides, or fragments thereof, may be obtained by any methods known in the art, including chemical synthesis and biological synthesis, as described elsewhere herein. In this regard, an exemplary human Cathepsin E sequence is SEQ ID NO.: 1. Antibodies produced in the inoculated animal which specifically bind to Cathepsin E, or fragments thereof, are then isolated from fluid obtained from the animal.

When the Nox3 inhibitor used in the compositions and methods of the invention is a polyclonal antibody (IgG), the antibody is generated by inoculating a suitable animal with a peptide comprising full length Nox3 protein (SEQ ID NO.: 76), or a fragment thereof, an upstream regulator, or fragments thereof. These polypeptides, or fragments thereof, may be obtained by any methods known in the art, including chemical synthesis and biological synthesis, as described elsewhere herein. In this regard, an exemplary human Nox3 sequence is SEQ ID NO.: 76. Antibodies produced in the inoculated animal which specifically bind to Nox3, or fragments thereof, are then isolated from fluid obtained from the animal.

Antibodies may be generated in this manner in several non-human mammals such as, but not limited to goat, sheep, horse, camel, rabbit, and donkey. Methods for generating polyclonal antibodies are well known in the art and are described, for example in Harlow, et al. (1998, In: Antibodies, A Laboratory Manual, Cold Spring Harbor, N.Y.).

Monoclonal antibodies directed against a full length Cathepsin E, Nox3, or fragments thereof, may be prepared using any well known monoclonal antibody preparation procedures, such as those described, for example, in Harlow et al. (1998, In: Antibodies, A Laboratory Manual, Cold Spring Harbor, N.Y.) and in Tuszynski et al. (1988, Blood, 72:109-115). Human monoclonal antibodies may be prepared by the method described in U.S. patent publication 2003/0224490. Monoclonal antibodies directed against an antigen are generated from mice immunized with the antigen using standard procedures as referenced herein. Nucleic acid encoding the monoclonal antibody obtained using the procedures described herein may be cloned and sequenced using technology which is available in the art, and is described, for example, in Wright et al. (1992, Critical Rev. in Immunol. 12(3,4):125-168) and the references cited therein.

When the antibody used in the methods of the invention is a biologically active antibody fragment or a synthetic antibody corresponding to antibody to a full length Cathepsin E, Nox3, or fragments thereof, the antibody is prepared as follows: a nucleic acid encoding the desired antibody or fragment thereof is cloned into a suitable vector. The vector is transfected into cells suitable for the generation of large quantities of the antibody or fragment thereof. DNA encoding the desired antibody is then expressed in the cell thereby producing the antibody. The nucleic acid encoding the desired peptide may be cloned and sequenced using technology which is available in the art, and described, for example, in Wright et al. (1992, Critical Rev. in Immunol. 12(3,4):125-168) and the references cited therein. Alternatively, quantities of the desired antibody or fragment thereof may also be synthesized using chemical synthesis technology. If the amino acid sequence of the antibody is known, the desired antibody can be chemically synthesized using methods known in the art as described elsewhere herein.

The present invention also includes the use of humanized antibodies specifically reactive with an epitope present on a target molecule. These antibodies are capable of binding to the target molecule. The humanized antibodies useful in the invention have a human framework and have one or more complementarity determining regions (CDRs) from an antibody, typically a mouse antibody, specifically reactive with a targeted cell surface molecule.

When the antibody used in the invention is humanized, the antibody can be generated as described in Queen, et al. (U.S. Pat. No. 6,180,370), Wright et al., (supra) and in the references cited therein, or in Gu et al. (1997, Thrombosis and Hematocyst 77(4):755-759), or using other methods of generating a humanized antibody known in the art. The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to humanized immunoglobulin coding sequences, including naturally-associated or heterologous promoter regions. The expression control sequences can be eukaryotic promoter systems in vectors capable of transforming or transfecting eukaryotic host cells, or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

Human constant region (CDR) DNA sequences from a variety of human cells can be isolated in accordance with well known procedures. Preferably, the human constant region DNA sequences are isolated from immortalized B-cells as described in WO 87/02671. CDRs useful in producing the antibodies of the present invention may be similarly derived from DNA encoding monoclonal antibodies capable of binding to the target molecule. Such humanized antibodies may be generated using well known methods in any convenient mammalian source capable of producing antibodies, including, but not limited to, mice, rats, camels, llamas, rabbits, or other vertebrates. Suitable cells for constant region and framework DNA sequences and host cells in which the antibodies are expressed and secreted, can be obtained from a number of sources, such as the American Type Culture Collection, Manassas, Va.

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies. Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species. As an example, the production of antibodies in mammals is detailed in such references as Harlow et al., (1998, Antibodies: A Laboratory Manual, Cold Spring Harbor, N.Y.).

V_{H} proteins isolated from other sources, such as animals with heavy chain disease (Seligmann et al., 1979, Immunological Rev. 48:145-167, incorporated herein by reference in its entirety), are also useful in the compositions and methods of the invention. The present invention further comprises variable heavy chain immunoglobulins produced from mice and other mammals, as detailed in Ward et al. (1989, Nature 341:544-546, incorporated herein by reference in its entirety). Briefly, V_{H} genes are isolated from mouse splenic preparations and expressed in E. coli. The present invention encompasses the use of such heavy chain immunoglobulins in the compositions and methods detailed herein.

Antibodies useful as Cathepsin E or Nox3 inhibitors in the invention may also be obtained from phage antibody libraries. To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (2001, Molecular Cloning: A Laboratory Manual, Cold Spring Harbor Laboratory Press, Cold Spring Harbor, N.Y.).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art and are described for example, in Wright et al., (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al., 1995, J. Mol. Biol. 248:97-105).

Once expressed, whole antibodies, dimers derived therefrom, individual light and heavy chains, or other forms of antibodies can be purified according to standard procedures known in the art. Such procedures include, but are not limited to, ammonium sulfate precipitation, the use of affinity columns, routine column chromatography, gel electrophoresis, and the like (see, generally, R. Scopes, “Protein Purification”, Springer-Verlag, N.Y. (1982)). Substantially pure antibodies of at least about 90% to 95% homogeneity are preferred, and antibodies having 98% to 99% or more homogeneity most preferred for pharmaceutical uses. Once purified, the antibodies may then be used to practice the method of the invention, or to prepare a pharmaceutical composition useful in practicing the method of the invention.

The antibodies of the present invention can be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g, Current Protocols in Molecular Biology, (Ausubel et al., eds.), Greene Publishing Associates and Wiley-Interscience, New York (2002)). Exemplary immunoassays are described briefly below (but are not intended to be in any way limiting).

2. Inhibitors of Cathepsin E or Nox3 Gene and mRNA Expression

a. Antisense Nucleic Acids

In one embodiment of the invention, an antisense nucleic acid sequence which is expressed by a plasmid vector is used to inhibit Cathepsin E or Nox3 expression. The antisense expressing vector is used to transfect a mammalian cell or the mammal itself, thereby causing reduced endogenous expression of Cathepsin E, or a regulator thereof, such as Nox3.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

b. Ribozymes

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In one embodiment of the invention, a ribozyme is used to inhibit Cathepsin E or Nox3 expression. Ribozymes useful for inhibiting the expression of a target molecule may be designed by incorporating target sequences into the basic ribozyme structure which are complementary, for example, to the mRNA sequence of Cathepsin E or Nox3 of the present invention. Ribozymes targeting Cathepsin E, or an upstream regulator thereof, may be synthesized using commercially available reagents (Applied Biosystems, Inc., Foster City, Calif.) or they may be genetically expressed from DNA encoding them.

c. siRNA

In one embodiment, siRNA is used to decrease the level of Cathepsin E protein. In another embodiment, siRNA is used to decrease the level of Nox3 protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, Pa. (2003); and Gregory J. Hannon, Ed., RNAi A Guide to Gene Silencing, Cold Spring Harbor Laboratory Press, Cold Spring Harbor, N.Y. (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of Cathepsin E protein using RNAi technology.

i. Modification of siRNA

Following the generation of the siRNA polynucleotide of the present invention, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987 Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide of the invention may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

ii. Vectors

In other related aspects, the invention includes an isolated nucleic acid encoding an inhibitor, wherein the inhibitor such as an siRNA, inhibits Cathepsin E, or a regulator thereof, operably linked to a nucleic acid comprising a promoter/regulatory sequence such that the nucleic acid is preferably capable of directing expression of the protein encoded by the nucleic acid. Thus, the invention encompasses expression vectors and methods for the introduction of exogenous DNA into cells with concomitant expression of the exogenous DNA in the cells such as those described, for example, in Sambrook et al. (2001, Molecular Cloning: A Laboratory Manual, Cold Spring Harbor Laboratory, New York), and in Ausubel et al. (1997, Current Protocols in Molecular Biology, John Wiley & Sons, New York). In another aspect of the invention, Cathepsin E, or a regulator thereof, can be inhibited by way of inactivating and/or sequestering Cathepsin E, or a regulator thereof. As such, inhibiting the effects of Cathepsin E can be accomplished by using a transdominant negative mutant.

In another aspect, the invention includes a vector comprising an siRNA polynucleotide. Preferably, the siRNA polynucleotide is capable of inhibiting the expression of a target polypeptide, wherein the target polypeptide is selected from the group consisting of Cathepsin E, or regulators thereof. The incorporation of a desired polynucleotide into a vector and the choice of vectors is well-known in the art as described in, for example, Sambrook et al., supra, and Ausubel et al., supra.

The siRNA polynucleotide can be cloned into a number of types of vectors. However, the present invention should not be construed to be limited to any particular vector. Instead, the present invention should be construed to encompass a wide plethora of vectors which are readily available and/or well-known in the art. For example, an siRNA polynucleotide of the invention can be cloned into a vector including, but not limited to a plasmid, a phagemid, a phage derivative, an animal viruses, and a cosmid. Vectors of particular interest include expression vectors, replication vectors, probe generation vectors, and sequencing vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

For expression of the siRNA, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 by upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 by apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. No. 4,683,202, U.S. Pat. No. 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2001). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

A promoter sequence exemplified in the experimental examples presented herein is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the invention should not be limited to the use of constitutive promoters. Inducible promoters are also contemplated as part of the invention. The use of an inducible promoter in the invention provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. Further, the invention includes the use of a tissue specific promoter, which promoter is active only in a desired tissue. Tissue specific promoters are well known in the art and include, but are not limited to, the HER-2 promoter and the PSA associated promoter sequences.

In order to assess the expression of the siRNA, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

3. Peptides

When the Cathepsin E or Nox3 inhibitor is a peptide, the peptide may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method, which utilizes tert-butyloxcarbonyl as the α-amino protecting group, and the FMOC method, which utilizes 9-fluorenylmethyloxcarbonyl to protect the α-amino of the amino acid residues. Both methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB (divinylbenzene), resin, which upon hydrofluoric acid (HF) treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by trifluoroacetic acid (TFA) in dicholoromethane. Esterification of the suitably activated carboxyl function, e.g. with dicyclohexylcarbodiimide (DCC), can then proceed by addition of the desired alcohol, followed by de-protection and isolation of the esterified peptide product.

Incorporation of N-terminal blocking groups may be achieved while the synthesized peptide is still attached to the resin, for instance by treatment with a suitable anhydride and nitrile. To incorporate an acetyl blocking group at the N-terminus, for instance, the resin-coupled peptide can be treated with 20% acetic anhydride in acetonitrile. The N-blocked peptide product may then be cleaved from the resin, de-protected and subsequently isolated.

Prior to its use as a Cathepsin E inhibitor in accordance with the invention, a peptide is purified to remove contaminants. Any one of a number of a conventional purification procedures may be used to attain the required level of purity including, for example, reversed-phase high-pressure liquid chromatography (HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can be also used to separate polypeptides based on their charge. Affinity chromatography is also useful in purification procedures.

Peptides may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

4. Small Molecules

When the Cathepsin E or Nox3 inhibitor is a small molecule, a small molecule activator may be obtained using standard methods known to the skilled artisan. Such methods include chemical organic synthesis or biological means. Biological means include purification from a biological source, recombinant synthesis and in vitro translation systems, using methods well known in the art.

Combinatorial libraries of molecularly diverse chemical compounds potentially useful in treating a variety of diseases and conditions are well known in the art as are method of making said libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

5. Identifying and Testing Candidate Cathepsin E or Nox3 Inhibitors

Cathepsin E or Nox3 inhibitors comprising inhibitors of gene expression, mRNA stability and expression, protein activity, function and expression of Cathepsin E, function and expression of Nox3, upstream regulators, and downstream effectors can be identified by screening test compounds. By way of a non-limiting example, inhibitors of endogenous Cathepsin E gene expression or of Cathepsin E mRNA expression can be identified by screening test compounds for their capacity to reduce or preclude Cathepsin E gene expression or Cathepsin E mRNA expression in a cell, preferably a pulmonary endothelial cell. The Cathepsin E coding sequence in such screening assays may include an in-frame fusion of a tag to the Cathepsin E coding sequence. Such tags enable monitoring of Cathepsin E expression by antibody detection of the tags or spectral methods of detection (e.g., fluorescence or luminescence).

Test compounds for use in such screening methods can be small molecules, nucleic acids including aptamers, peptides, peptidomimetics and other drugs. Peptide fragments are contemplated that can competitively inhibit, for example, the binding of full length Cathepsin E to a downstream effector molecule, thereby inhibiting Cathepsin E activity.

The test compounds of the present invention can be obtained using any of the numerous approaches in combinatorial library methods known in the art, including biological libraries, spatially-addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam, 1997, Anticancer Drug Des. 12:145). Inhibitors of Cathepsin E expression may be useful in therapeutic applications, or serve as lead drugs in the development of therapeutics. Synthetic techniques may be used to produce compounds, such as: chemical and enzymatic production of small molecules, peptides, nucleic acids, antibodies, and other therapeutic compositions useful in the practice of the methods of the invention. Other techniques may be used which are not described herein, but are known to those of skill in the art.

B. Toll-Like Receptor 4 (TLR4) Activators

1. TLR4 Agonists

TLR4 is a member of the toll-like receptors superfamily, a class of pattern recognition receptors (PRR) comprising single membrane-spanning non-catalytic receptors that recognize molecules that are broadly shared by pathogens but distinguishable from host molecules, collectively referred to as pathogen-associated molecular patterns.

When the TLR4 activator is a TLR4 agonist that binds to TLR4, TLR4 recruit adapter protein molecules within the cytoplasm of cells in order to propagate a signal. Four adapter protein molecules are known to be involved in TLR signaling: MyD88, Tirap (also called Mal), Trif, and Tram. The adapters activate other molecules within the cell, including certain protein kinases (IRAK1, IRAK4, TBK1, and IKKi) that amplify the signal, and ultimately lead to the induction or suppression of genes that orchestrate the desired response.

Naturally-occurring TLR4 agonists can be molecules associated with microbial threats to an organism (i.e. pathogen or cell stress) and are highly specific to these threats (i.e. cannot be mistaken for self molecules). Examples of well-conserved features in pathogens that act as TLR agonists include bacterial cell-surface lipopolysaccharides (LPS), lipoproteins, lipopeptides and lipoarabinomannan; proteins such as flagellin from bacterial flagella; double-stranded RNA of viruses or the unmethylated CpG islands of bacterial and viral DNA, and certain other RNA and DNA (Table I). TLR agonists may also be synthetic molecules, provided that they specifically bind a TLR4 and supress Nox3 and Cathepsin E expression or activity in lung.

TLR4 agonists useful in the invention are well known in the art. Naturally occurring TLR4 ligands include lipopolysaccharides, viral glycoproteins, mycobacterial glycolipid lipoarrabinomannan (LAM), bacterial lipoproteins, peptidoglycans, zymosan (Akira et al., 2001, Nature Immunol 2:675-680; Aderem and Ulevitch, 2000, Nature 406:782-787) and Enterobacterial LPS.

Hyaluronan (HA) is a potential endogenous TLR4 ligand. HA is a glycosaminoglycan polymer that is present in all tissues and a major component of lung extracellular matrix. Aerosolized HA protects against elastase- and cigarette smoke-induced emphysema (Cantor et al., 2005, Exp. Lung Res. 31:417-430). CD44 is a major cell surface HA receptor, however, HA can also signal through TLR4 in endothelial (Taylor et al., 2004, J. Biol. Chem. 279:17079-17084) as well as in lung epithelial cells (Jianag et al., 2005, Nature Med. 11:1173-1179).

Because TLR4s are pattern recognition receptors and the structure of their agonists are well known, the invention shall not be construed to be limited to those agonists recited herein, but should be construed to encompass any compound, small molecule, peptide, or nucleic acid that specifically binds to a TLR4 and is able to inhibit Nox3 or Cathepsin E expression or activity. New TLR4 agonists may be discovered using standard screening techniques well-known in the art. Test compounds for use in such screening methods can be small molecules, peptides, nucleic acids, or other drugs.

Known TLR4 agonists are commercially available. In addition, TLR4 agonists may be obtained using standard methods known to the skilled artisan. Such methods include chemical organic synthesis or biological means. Biological means include purification from a biological source, recombinant synthesis and in vitro translation systems, using methods well known in the art.

2. Peptides

When the TLR4 activator is a peptide, the peptide may be chemically synthesized or modified as described elsewhere herein.

3. Nucleic Acids

When the TLR4 activator comprises a nucleic acid, any number of procedures may be used for the generation of an isolated nucleic acid encoding the agonist as well as derivative or variant forms of the isolated nucleic acid, using recombinant DNA methodology well known in the art (see Sambrook et al., 2001, Molecular Cloning: A Laboratory Manual, Cold Spring Harbor Laboratory Press, New York; Ausubel et al., 2001, Current Protocols in Molecular Biology, Green & Wiley, New York) and by direct synthesis. For recombinant and in vitro transcription, DNA encoding RNA molecules can be obtained from known clones of TLR4 activator, by synthesizing a DNA molecule encoding an RNA molecule, or by cloning the gene encoding the RNA molecule. Techniques for in vitro transcription of RNA molecules and methods for cloning genes encoding known RNA molecules are described by, for example, Sambrook et al.

An isolated nucleic acid of the present invention can be produced using conventional nucleic acid synthesis or by recombinant nucleic acid methods known in the art and described elsewhere herein (Sambrook et al., 2001, Molecular Cloning: A Laboratory Manual, Cold Spring Harbor Laboratory Press, New York) and Ausubel et al. (2001, Current Protocols in Molecular Biology, Green & Wiley, New York).

As an example, a method for synthesizing nucleic acids de novo involves the organic synthesis of a nucleic acid from nucleoside derivatives. This synthesis may be performed in solution or on a solid support. One type of organic synthesis is the phosphotriester method, which has been used to prepare gene fragments or short genes. In the phosphotriester method, oligonucleotides are prepared which can then be joined together to form longer nucleic acids. For a description of this method, see Narang et al., (1979, Meth. Enzymol., 68: 90) and U.S. Pat. No. 4,356,270. The phosphotriester method can be used in the present invention to synthesize an isolated TLR4 activator nucleic acid.

In addition, the compositions of the present invention can be synthesized in whole or in part, or an isolated TLR4 activator nucleic acid can be conjugated to another nucleic acid using organic synthesis such as the phosphodiester method, which has been used to prepare a tRNA gene. See Brown et al. (1979, Meth. Enzymol., 68: 109) for a description of this method. As in the phosphotriester method, the phosphodiester method involves synthesis of oligonucleotides which are subsequently joined together to form the desired nucleic acid.

A third method for synthesizing nucleic acids, described in U.S. Pat. No. 4,293,652, is a hybrid of the above-described organic synthesis and molecular cloning methods. In this process, the appropriate number of oligonucleotides to make up the desired nucleic acid sequence is organically synthesized and inserted sequentially into a vector which is amplified by growth prior to each succeeding insertion.

In addition, molecular biological methods, such as using a nucleic acid as a template for a PCR or LCR reaction, or cloning a nucleic acid into a vector and transforming a cell with the vector can be used to make large amounts of the nucleic acid of the present invention.

TLR4 activators may include small synthetic nucleic acid compounds. Thus, oligonucleotide agents are incorporated herein and include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, preferably as occur naturally in the human body. The art has referred to rare or unusual, but naturally occurring, RNAs as modified RNAs, see, e.g., Limbach et al. (1994, Nucleic Acids Res. 22: 2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, preferably different from that which occurs in the human body.

As nucleic acids are polymers of subunits or monomers, many of the modifications described below occur at a position which is repeated within a nucleic acid, e.g., a modification of a base, or a phosphate moiety, or a non-linking O of a phosphate moiety. In some cases the modification will occur at all of the subject positions in the nucleic acid but in many, and in fact in most cases it will not. By way of example, a modification may only occur at a 3′ or 5′ terminal position, in a terminal region, e.g., at a position on a terminal nucleotide, or in the last 2, 3, 4, 5, or 10 nucleotides of a strand. A component can be attached at the 3′ end, the 5′ end, or at an internal position, or at a combination of these positions. For example, the component can be at the 3′ end and the 5′ end; at the 3′ end and at one or more internal positions; at the 5′ end and at one or more internal positions; or at the 3′ end, the 5′ end, and at one or more internal positions. For example, a phosphorothioate modification at a non-linking O position may only occur at one or both termini, or may only occur in a terminal region, e.g., at a position on a terminal nucleotide or in the last 2, 3, 4, 5, or nucleotides of the oligonucleotide. The 5′ end can be phosphorylated.

For increased nuclease resistance and/or binding affinity to the target, an oligonucleotide agent, can include, for example, T-modified ribose units and/or phosphorothioate linkages. E.g., the 2′ hydroxyl group (OH) can be modified or replaced with a number of different “oxy” or “deoxy” substituents.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; amine, O-AMINE and aminoalkoxy, O(CH₂)ₙAMINE, (e.g., AMINE=NH₂; alkylamino, dialkylamino, heterocyclyl amino, arylamino, diaryl amino, heteroaryl amino, or diheteroaryl amino, ethylene diamine, polyamino). It is noteworthy that oligonucleotides containing only the methoxyethyl group (MOE), (OCH₂CH₂OCH₃, a PEG derivative), exhibit nuclease stabilities comparable to those modified with the robust phosphorothioate modification.

Preferred substitutents include but are not limited to 2′-methoxyethyl, 2′-OCH3, 2′-O-allyl, 2′-C— allyl, and 2′-fluoro.

“Deoxy” modifications include hydrogen (i.e. deoxyribose sugars); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl amino, arylamino, diaryl amino, heteroaryl amino, or diheteroaryl amino), —NHC(O)R(R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality.

One way to increase resistance is to identify cleavage sites and modify such sites to inhibit cleavage. For example, the dinucleotides 5′-UA-3′,5′-UG-3′,5′-CA-3′,5′-UU-3′, or 5′-CC-3′ can serve as cleavage sites. Enhanced nuclease resistance can therefore be achieved by modifying the 5′ nucleotide, resulting, for example, in at least one 5′-uridine-adenine-3′ (5′-UA-3′) dinucleotide wherein the uridine is a 2′-modified nucleotide; at least one 5′-uridine-guanine-3′ (5′-UG-3′) dinucleotide, wherein the 5′-uridine is a 2′-modified nucleotide; at least one 5′-cytidine-adenine-3′ (5′-CA-3′) dinucleotide, wherein the 5′-cytidine is a 2′-modified nucleotide; at least one 5′-uridine-uridine-3′ (5′-UU-3′) dinucleotide, wherein the 5′-uridine is a 2′-modified nucleotide; or at least one 5′-cytidine-cytidine-3′ (5′-CC-3′) dinucleotide, wherein the 5′-cytidine is a 2′-modified nucleotide. In certain embodiments, all the pyrimidines of the miRNA inhibitor carry a 2′-modification, and the miRNA inhibitor therefore has enhanced resistance to endonucleases.

In addition, to increase nuclease resistance, the 2′ modifications can be used in combination with one or more phosphate linker modifications (e.g., phosphorothioate). The so-called “chimeric” oligonucleotides are those that contain two or more different modifications.

With respect to phosphorothioate linkages that serve to increase protection against RNase activity, the miRNA inhibitor can include a phosphorothioate at least the first, second, or third internucleotide linkage at the 5′ or 3′ end of the nucleotide sequence. In one embodiment, the miRNA inhibitor includes a 2′-modified nucleotide, e.g., a 2′-deoxy, 2′-deoxy-2′-fluoro, 2′-O-methyl, 2′-O -methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O -dimethylaminoethyloxyethyl (2′-O-DMAEOE), or 2′-O—N-methylacetamido (2′-O-NMA). In a preferred embodiment, the miRNA inhibitor includes at least one 2′-O -methyl-modified nucleotide, and in some embodiments, all of the nucleotides of the miRNA inhibitor include a 2′-O-methyl modification.

The 5′-terminus can be blocked with an aminoalkyl group, e.g., a 5′-O-alkylamino substituent. Other 5′ conjugates can inhibit 5′-3′ exonucleolytic cleavage. While not being bound by theory, a 5′ conjugate, such as naproxen or ibuprofen, may inhibit exonucleolytic cleavage by sterically blocking the exonuclease from binding to the 5′ end of the oligonucleotide. Even small alkyl chains, aryl groups, or heterocyclic conjugates or modified sugars (D-ribose, deoxyribose, glucose etc.) can block 3′-5′-exonucleases.

The oligonucleotide can be constructed using chemical synthesis and/or enzymatic ligation reactions using procedures known in the art. For example, an oligonucleotide can be chemically synthesized using naturally occurring nucleotides or variously modified nucleotides designed to increase the biological stability of the molecules or to increase the physical stability of the duplex formed between the oligonucleotide and target nucleic acids, e.g., phosphorothioate derivatives and acridine substituted nucleotides can be used. Other appropriate nucleic acid modifications are described herein. Alternatively, the oligonucleotide can be produced biologically using an expression vector into which a nucleic acid has been subcloned in an antisense orientation (i.e., RNA transcribed from the inserted nucleic acid will be of an antisense orientation to a target nucleic acid of interest (e.g., an mRNA, pre-mRNA, or an miRNA).

Any polynucleotide of the invention may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

4. Small Molecules

When the TLR4 activator is a small molecule, a small molecule activator may be obtained using standard methods known to the skilled artisan, and described elsewhere herein.

5. Identifying and Testing Candidate TLR4 Activators

A test compound useful in the present invention is a potential TLR4 activator and may be a peptide, a nucleic acid, a small molecule, or other drug that specifically binds to or activates TLR4, increases TLR4 expression, stability or function, or regulates TLR4 downstream effectors. For example, a TLR4 activator might inhibit Nox3 or Cathepsin E expression or activity. Test molecules may be obtained using any of the numerous approaches in combinatorial library methods known in the art, including biological libraries, spatially-addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam, 1997, Anticancer Drug Des. 12:145).

Examples of methods for the synthesis of molecular libraries may be found in the art, for example, in: DeWitt et al., 1993, Proc. Natl. Acad. Sci. USA 90:6909-6913; Erb et al., 1994, Proc. Natl. Acad. Sci. USA 91:11422-11426; Zuckermann et al., 1994, J. Med. Chem. 37:2678-2685; Cho et al., 1992, Science 261:1303-1305; Carell et al., 1994, Angew. Chem. Int. Ed. Engl. 33:2059-2061; Carell et al., 1994, Angew. Chem. Int. Ed. Engl. 33:2061-2064; and Gallop et al., 1994, J. Med. Chem. 37:1233-1251.

Libraries of compounds may be presented in solution (e.g., Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869), or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310).

The resulting libraries of candidate molecules may be screened to determine their efficacy as TLR4 activators using any technique well known in the art. Such techniques include, but are not limited to, high-throughput bioassays, such as binding assays or activity based assays, to determine a molecule's ability to specifically bind to or activate a TLR; structural analysis such as X-ray crystallography; drug fragment-based analysis, including binding assays; computational analysis (e.g. Target Infomatics Platform, Eidogen; Passadena, Calif.); animal-based, tissue-based, or cell-based assays, to determine a molecule's effect on as a TLR4 activator.

### II. Methods

A. Methods of Detecting an Individual with Emphysema/COPD or an Individual At-Risk of Developing Emphysema/COPD

In particular embodiments, the diagnostic methods of the invention comprise collecting a sample from a patient, contacting the sample with at least one antibody specific for Cathepsin E, and detecting antibody binding thereto. Samples that contain elevated Cathepsin E identify an individual at risk of experiencing hyperglycemia whether or riot during pregnancy, an individual with emphysema/COPD or an individual at-risk of developing emphysema/COPD.

Any methods available in the art for identification or detection of Cathepsin E are encompassed herein. Cathepsin E can be detected at a nucleic acid level or a protein level. In order to determine up-regulation of Cathepsin E expression, levels of the Cathepsin E are measured in the body sample to be examined and compared with a corresponding body sample that originates from a normal, not-at-risk individual. In another embodiment of the invention, up-regulation of Cathepsin E is determined by measuring levels of Cathepsin E in the body sample to be examined and comparing with an average value obtained from more than one not-at-risk individuals. In still another embodiment of the invention, up-regulation of Cathepsin E is determined by measuring levels of Cathepsin E in the body sample to be examined and comparing with levels of Cathepsin E obtained from a body sample obtained from the same individual at a different time.

Methods for detecting Cathepsin E comprise any method that determines the quantity or the presence of Cathepsin E either at the nucleic acid or protein level. Such methods are well known in the art and include but are not limited to western blots, northern blots, southern blots, ELISA, immunoprecipitation, immunofluorescence, flow cytometry, immunocytochemistry, nucleic acid hybridization techniques, nucleic acid reverse transcription methods, and nucleic acid amplification methods. In particular embodiments, Cathepsin E is detected on a protein level using, for example, antibodies that are directed against Cathepsin E protein. These antibodies can be used in various methods such as Western blot, ELISA, immunoprecipitation, or immunocytochemistry techniques.

The invention should not be limited to any one method of protein or nucleic acid detection method recited herein, but rather should encompass all known or heretofore unknown methods of detection as are, or become, known in the art.

### 1. Methods of Detecting Cathepsin E Protein

In one embodiment, antibodies specific for Cathepsin E protein are used to detect Cathepsin E protein in a body sample. The method comprises obtaining a body sample from a patient, contacting the body sample with at least one antibody directed to a Cathepsin E to determine if Cathepsin E is up-regulated in the patient sample. One of skill in the art will recognize that the immunocytochemistry method described herein below is performed manually or in an automated fashion.

Samples may need to be modified in order to render the Cathepsin E antigens accessible to antibody binding. In a particular aspect of the immunocytochemistry methods, slides are transferred to a pretreatment buffer, for example phosphate buffered saline containing Triton-X. Incubating the sample in the pretreatment buffer rapidly disrupts the lipid bilayer of the cells and renders the antigens (i.e., biomarker proteins) more accessible for antibody binding. The pretreatment buffer may comprise a polymer, a detergent, or a nonionic or anionic surfactant such as, for example, an ethyloxylated anionic or nonionic surfactant, an alkanoate or an alkoxylate or even blends of these surfactants or even the use of a bile salt. The pretreatment buffers of the invention are used in methods for making antigens more accessible for antibody binding in an immunoassay, such as, for example, an immunocytochemistry method or an immunohistochemistry method.

Any method for making antigens more accessible for antibody binding may be used in the practice of the invention, including antigen retrieval methods known in the art. See, for example, Bibbo, 2002, Acta. Cytol. 46:25 29; Saqi, 2003, Diagn. Cytopathol. 27:365 370; Bibbo, 2003, Anal. Quant. Cytol. Histol. 25:8 11. In some embodiments, antigen retrieval comprises storing the slides in 95% ethanol for at least 24 hours, immersing the slides one time in Target Retrieval Solution pH 6.0 (DAKO S1699)/dH2O bath preheated to 95° C., and placing the slides in a steamer for 25 minutes.

Following pretreatment or antigen retrieval to increase antigen accessibility, samples are blocked using an appropriate blocking agent, e.g., a peroxidase blocking reagent such as hydrogen peroxide. In some embodiments, the samples are blocked using a protein blocking reagent to prevent non-specific binding of the antibody. The protein blocking reagent may comprise, for example, purified casein, serum or solution of milk proteins. An antibody directed to a Cathepsin E is then incubated with the sample.

Techniques for detecting antibody binding are well known in the art. Antibody binding to Cathepsin E may be detected through the use of chemical reagents that generate a detectable signal that corresponds to the level of antibody binding and, accordingly, to the level of Cathepsin E protein expression. In one of the preferred immunocytochemistry methods of the invention, antibody binding is detected through the use of a secondary antibody that is conjugated to a labeled polymer. Examples of labeled polymers include but are not limited to polymer-enzyme conjugates. The enzymes in these complexes are typically used to catalyze the deposition of a chromogen at the antigen-antibody binding site, thereby resulting in cell staining that corresponds to expression level of the biomarker of interest. Enzymes of particular interest include horseradish peroxidase (HRP) and alkaline phosphatase (AP). Commercial antibody detection systems, such as, for example the Dako Envision+ system (Dako North America, Inc., Carpinteria, Calif.) and Mach 3 system (Biocare Medical, Walnut Creek, Calif.), may be used to practice the present invention.

In one particular immunocytochemistry method of the invention, antibody binding to a biomarker is detected through the use of an HRP-labeled polymer that is conjugated to a secondary antibody. Antibody binding can also be detected through the use of a mouse probe reagent, which binds to mouse monoclonal antibodies, and a polymer conjugated to HRP, which binds to the mouse probe reagent. Slides are stained for antibody binding using the chromogen 3,3-diaminobenzidine (DAB) and then counterstained with hematoxylin and, optionally, a bluing agent such as ammonium hydroxide or TBS/Tween-20. In some aspects of the invention, slides are reviewed microscopically by a cytotechnologist and/or a pathologist to assess cell staining (i.e., biomarker overexpression). Alternatively, samples may be reviewed via automated microscopy or by personnel with the assistance of computer software that facilitates the identification of positive staining cells.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

In regard to detection of antibody staining in the immunocytochemistry methods of the invention, there also exist in the art video-microscopy and software methods for the quantitative determination of an amount of multiple molecular species (e.g., biomarker proteins) in a biological sample, wherein each molecular species present is indicated by a representative dye marker having a specific color. Such methods are also known in the art as colorimetric analysis methods. In these methods, video-microscopy is used to provide an image of the biological sample after it has been stained to visually indicate the presence of a particular biomarker of interest. Some of these methods, such as those disclosed in U.S. patent application Ser. No. 09/957,446 and U.S. patent application Ser. No. 10/057,729 to Marcelpoil., incorporated herein by reference, disclose the use of an imaging system and associated software to determine the relative amounts of each molecular species present based on the presence of representative color dye markers as indicated by those color dye markers' optical density or transmittance value, respectively, as determined by an imaging system and associated software. These techniques provide quantitative determinations of the relative amounts of each molecular species in a stained biological sample using a single video image that is “deconstructed” into its component color parts.

The antibodies used to practice the invention are selected to have high specificity for Cathepsin E protein of interest. Methods for making antibodies and for selecting appropriate antibodies are known in the art. See, for example, Celis, J. E. ed. (in press) Cell Biology & Laboratory Handbook, 3rd edition (Academic Press, New York), which is herein incorporated in its entirety by reference. In some embodiments, commercial antibodies directed to specific biomarker proteins may be used to practice the invention. The antibodies of the invention may be selected on the basis of desirable staining of cytological, rather than histological, samples. That is, in particular embodiments the antibodies are selected with the end sample type (i.e., cytology preparations) in mind and for binding specificity.

One of skill in the art will recognize that optimization of antibody titer and detection chemistry is needed to maximize the signal to noise ratio for a particular antibody. Antibody concentrations that maximize specific binding to the biomarkers of the invention and minimize non-specific binding (or “background”) will be determined in reference to the type of biological sample being tested. In particular embodiments, appropriate antibody titers for use cytology preparations are determined by initially testing various antibody dilutions on formalin-fixed paraffin-embedded normal tissue samples. Optimal antibody concentrations and detection chemistry conditions are first determined for formalin-fixed paraffin-embedded tissue samples. The design of assays to optimize antibody titer and detection conditions is standard and well within the routine capabilities of those of ordinary skill in the art. After the optimal conditions for fixed tissue samples are determined, each antibody is then used in cytology preparations under the same conditions. Some antibodies require additional optimization to reduce background staining and/or to increase specificity and sensitivity of staining in the cytology samples.

Furthermore, one of skill in the art will recognize that the concentration of a particular antibody used to practice the methods of the invention will vary depending on such factors as time for binding, level of specificity of the antibody for the Cathepsin E protein, and method of body sample preparation. Furthermore, the detection chemistry used to visualize antibody binding to a Cathepsin E protein must also be optimized to produce the desired signal to noise ratio.

### Immunoassays

Immunoassays, in their simplest and most direct sense, are binding assays. Certain preferred immunoassays are the various types of enzyme linked immunosorbent assays (ELISA) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, antibodies binding to the Cathepsin E proteins of the invention are immobilized onto a selected surface exhibiting protein affinity, such as a well in a polystyrene microtiter plate. Then, a test composition suspected of containing the biomarker antigen, such as a clinical sample, is added to the wells. After binding and washing to remove non-specifically bound immunecomplexes, the bound antibody may be detected. Detection is generally achieved by the addition of a second antibody specific for the target protein, that is linked to a detectable label. This type of ELISA is a simple “sandwich ELISA”. Detection may also be achieved by the addition of a second antibody, followed by the addition of a third antibody that has binding affinity for the second antibody, with the third antibody being linked to a detectable label.

In another exemplary ELISA, the samples suspected of containing the Cathepsin E antigen are immobilized onto the well surface and then contacted with the antibodies of the invention. After binding and washing to remove non-specifically bound immunecomplexes, the bound antigen is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Another ELISA in which the proteins or peptides are immobilized, involves the use of antibody competition in the detection. In this ELISA, labeled antibodies are added to the wells, allowed to bind to the Cathepsin E protein, and detected by means of their label. The amount of marker antigen in an unknown sample is then determined by mixing the sample with the labeled antibodies before or during incubation with coated wells. The presence of Cathepsin E antigen in the sample acts to reduce the amount of antibody available for binding to the well and thus reduces the ultimate signal. This is appropriate for detecting antibodies in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immunecomplexes. These are described as follows:

In coating a plate with either antigen or antibody, the wells of the plate are incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating of nonspecific adsorption sites on the immobilizing surface reduces the background caused by nonspecific binding of antisera to the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control and/or clinical or biological sample to be tested under conditions effective to allow immunecomplex (antigen/antibody) formation. Detection of the immunecomplex then requires a labeled secondary binding ligand or antibody, or a secondary binding ligand or antibody in conjunction with a labeled tertiary antibody or third binding ligand.

“Under conditions effective to allow immunecomplex (antigen/antibody) formation” means that the conditions preferably include diluting the antigens and antibodies with solutions such as, but not limited to, BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hours, at temperatures preferably on the order of 25° to 27° C., or may be overnight at about 4° C.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this label is an enzyme that generates a color or other detectable signal upon incubating with an appropriate chromogenic or other substrate. Thus, for example, the first or second immunecomplex can be detected with a urease, glucose oxidase, alkaline phosphatase or hydrogen peroxidase-conjugated antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation is then achieved by measuring the degree of color generation, e.g., using a visible spectra spectrophotometer.

2. Nucleic Acid-Based Techniques

In other embodiments, the expression of Cathepsin E is detected at the nucleic acid level. Nucleic acid-based techniques for assessing expression are well known in the art and include, for example, determining the level of Cathepsin E mRNA in a body sample. Many expression detection methods use isolated RNA. Any RNA isolation technique that does not select against the isolation of mRNA can be utilized for the purification of RNA from body samples (see, e.g., Ausubel, ed., 1999, Current Protocols in Molecular Biology (John Wiley & Sons, New York). Additionally, large numbers of tissue samples can readily be processed using techniques well known to those of skill in the art, such as, for example, the single-step RNA isolation process of Chomczynski, 1989, U.S. Pat. No. 4,843,155).

The term “probe” refers to any molecule that is capable of selectively binding to a specifically intended target biomolecule, for example, a nucleotide transcript or a protein encoded by or corresponding to Cathepsin E. Probes can be synthesized by one of skill in the art, or derived from appropriate biological preparations. Probes may be specifically designed to be labeled with a detectable label. Examples of molecules that can be used as probes include, but are not limited to, RNA, DNA, proteins, antibodies, and organic molecules.

Isolated mRNA can be detected in hybridization or amplification assays that include, but are not limited to, Southern or Northern analyses, polymerase chain reaction analyses and probe arrays. One method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding Cathepsin E of the present invention. Hybridization of an mRNA with the probe indicates that the Cathepsin E in question is being expressed.

In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array (Santa Clara, Calif.). A skilled artisan can readily adapt known mRNA detection methods for use in detecting the level of mRNA encoded by the biomarkers of the present invention.

An alternative method for determining the level of Cathepsin E mRNA in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189 193), self sustained sequence replication (Guatelli, 1990, Proc. Natl. Acad. Sci. USA, 87:1874 1878), transcriptional amplification system (Kwoh, 1989, Proc. Natl. Acad. Sci. USA, 86:1173 1177), Q-Beta Replicase (Lizardi, 1988, Bio/Technology, 6:1197), rolling circle replication (Lizardi, U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, biomarker expression is assessed by quantitative fluorogenic RT-PCR (i.e., the TaqMan® System). Such methods typically use pairs of oligonucleotide primers that are specific for the biomarker of interest. Methods for designing oligonucleotide primers specific for a known sequence are well known in the art.

Cathepsin E expression levels of RNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The detection of biomarker expression may also comprise using nucleic acid probes in solution.

### Preparation of Nucleic Acid Probes

Using the sequence information provided herein, the nucleic acids may be synthesized according to a number of standard methods known in the art. Oligonucleotide synthesis, is carried out on commercially available solid phase oligonucleotide synthesis machines or manually synthesized using the solid phase phosphoramidite triester method described by Beaucage, 1981, Tetrahedron Letters, 22: 1859-1862.

Once a nucleic acid encoding a biomarker is synthesized, it may be amplified and/or cloned according to standard methods in order to produce recombinant polypeptides. Molecular cloning techniques to achieve these ends are known in the art. A wide variety of cloning and in vitro amplification methods suitable for the construction of recombinant nucleic acids are known to those skilled in the art.

Examples of techniques sufficient to direct persons of skill through in vitro amplification methods, including the polymerase chain reaction (PCR), the ligase chain reaction (LCR), and other DNA or RNA polymerase-mediated techniques are found in Sambrook, 2001, Molecular Cloning: A Laboratory Manuel, 3^{rd} ed. (Cold Spring Harbor Laboratory Press, Cold Spring Harbor, N.Y.

Once the nucleic acid for a biomarker is cloned, a skilled artisan may express the recombinant gene(s) in a variety of engineered cells. Examples of such cells include bacteria, yeast, filamentous fungi, insect (especially employing baculoviral vectors), and mammalian cells. It is expected that those of skill in the art are knowledgeable in the numerous expression systems available for expressing the biomarker proteins of the invention.

B. Methods of Treating or Preventing COPD/Emphysema

The present invention provides a method of treating emphysema/COPD in a mammal. In one embodiment, the present invention provides a method of preventing a mammal at-risk of developing emphysema/COPD from developing pathophysiological changes and clinical sequalae associated with emphysema/COPD.

The method of the invention comprises administering a therapeutically effective amount of at least one TLR4 activator, at least one Cathepsin E inhibitor, or a combination thereof, to a mammal wherein a TLR4 activator, a Cathepsin E inhibitor, or combination thereof prevents, attenuates, or halts the pathophysiological changes associated with dysregulation of the TLR4 pathway in lung, including but not limited to decreased TLR4 expression, increased Cathepsin E expression, increased lung cell death, increased lung volume, alveolar destruction, and decreased lung elasticity.

In another embodiment, the method of the invention comprises administering a therapeutically effective amount of at least one TLR4 activator, at least one Cathepsin E inhibitor, or a combination thereof, to a mammal wherein a TLR4 activator, a Cathepsin E inhibitor, or combination thereof is used to treat a mammal diagnosed with a disease or disorder wherein dysregulation of the TLR4 pathway in lung is a component of the disease or disorder.

In still another embodiment, the method of the invention comprises administering a therapeutically effective amount of at least one TLR4 activator, at least one Cathepsin E inhibitor, or a combination thereof, is used to treat emphysema/COPD.

In still another embodiment, the method of the invention comprises administering a therapeutically effective amount of at least one TLR4 activator, at least one Cathepsin E inhibitor, or a combination thereof; is used to prevent the development of emphysema/COPD in an individual at-risk of developing emphysema/COPD.

The subject may be diagnosed with a disease or disorder wherein the disease or disorder has a dysregulation of the TLR4 pathway in lung as part of the disease's clinical features. Alternatively, the subject may be at-risk of developing a disease or disorder wherein the disease or disorder has a dysregulation of the TLR4 pathway in lung as part of the disease's clinical features. Examples of a disease or disorder which may be treated using the methods of the present invention include but are not limited to chronic obstructive pulmonary disease (COPD) and emphysema. In a preferred embodiment the subject is a mammal. In a more preferred embodiment the subject is a human.

Methods of prophylaxis (i.e., prevention or decreased risk of disease), as well as reduction in the frequency or severity of symptoms associated with emphysema/COPD or any related disease or disorder, are encompassed by the present invention.

The method of the invention comprises administering a therapeutically effective amount of at least one TLR4 activator, at least one Cathepsin E inhibitor, or a combination thereof, to a mammal wherein a composition of the present invention comprising a TLR4 activator, a Cathepsin E inhibitor, or a combination thereof is used either alone or in combination with other therapeutic agents to treat a subject. A TLR4 activator, a Cathepsin E inhibitor, or a combination thereof may be administered either, before, during, after, or throughout the administration of said therapeutic agent. The compositions and methods of the present invention can be used in combination with other treatment regimens, including virostatic and virotoxic agents, antibiotic agents, antifungal agents, anti-inflammatory agents (steroidal and non-steroidal), antidepressants, anxiolytics, pain management agents, (acetaminophen, aspirin, ibuprofen, opiates (including morphine, hydrocodone, codeine, fentanyl, methadone), steroids (including prednisone and dexamethasone), and antidepressants (including gabapentin, amitriptyline, imipramine, doxepin) antihistamines, antitussives, muscle relaxants, bronchodilators, beta-agonists, anticholinergics, corticosteroids, mast cell stabilizers, leukotriene modifiers, methylxanthines, as well as combination therapies, and the like. The invention can also be used in combination with other treatment modalities, such as chemotherapy, cryotherapy, hyperthermia, radiation therapy, and the like.

C. Methods of Delivering a Cathepsin E Inhibitor or Tlr4 Activator to a Cell

The present invention comprises a method for treating or preventing the development of emphysema/COPD in a mammal, said method comprising administering a therapeutic amount of a TLR4 activator, a Cathepsin E inhibitor, or a combination thereof to said mammal. In particular, the invention includes a method for attenuating lung cell apoptosis, alveolar destruction, increased lung volume, reduced lung elasticity, all of which are features of emphysema/COPD.

Isolated nucleic acid-based TLR4 activators or Cathepsin E inhibitors can be delivered to a cell in vitro or in vivo using viral vectors comprising one or more isolated TLR4 activator or Cathepsin E inhibitor sequences. Generally, the nucleic acid sequence has been incorporated into the genome of the viral vector. The viral vector comprising an isolated TLR4 activator or Cathepsin E inhibitor nucleic acid described herein can be contacted with a cell in vitro or in vivo and infection can occur. The cell can then be used experimentally to study, for example, the effect of an isolated TLR4 activator or Cathepsin E inhibitor in vitro, or the cells can be implanted into a subject for therapeutic use. The cell can be migratory, such as a hematopoietic cell, or non-migratory. The cell can be present in a biological sample obtained from the subject (e.g., blood, bone marrow, tissue, fluids, organs, etc.) and used in the treatment of disease, or can be obtained from cell culture.

After contact with the viral vector comprising an isolated TLR4 activator or Cathepsin E inhibitor nucleic acid sequence, the sample can be returned to the subject or re-administered to a culture of subject cells according to methods known to those practiced in the art. In the case of delivery to a subject or experimental animal model (e.g., rat, mouse, monkey, chimpanzee), such a treatment procedure is sometimes referred to as ex vivo treatment or therapy. Frequently, the cell is removed from the subject or animal and returned to the subject or animal once contacted with the viral vector comprising the isolated TLR4 activator or Cathepsin E inhibitor nucleic acid of the present invention. Ex vivo gene therapy has been described, for example, in Kasid et al., Proc. Natl. Acad. Sci. USA 87:473 (1990); Rosenberg et al, New Engl. J. Med. 323:570 (1990); Williams et al., Nature 310476 (1984); Dick et al., Cell 42:71 (1985); Keller et al., Nature 318:149 (1985) and Anderson et al., U.S. Pat. No. 5,399,346 (1994).

Where a cell is contacted in vitro, the cell incorporating the viral vector comprising an isolated TLR4 activator or Cathepsin E inhibitor nucleic acid can be implanted into a subject or experimental animal model for delivery or used in in vitro experimentation to study cellular events mediated by TLR4 activator or Cathepsin E inhibitor activity.

Various viral vectors can be used to introduce an isolated TLR4 activator or Cathepsin E inhibitor nucleic acid into mammalian cells. Viral vectors include retrovirus, adenovirus, parvovirus (e.g., adeno-associated viruses), coronavirus, negative-strand RNA viruses such as orthomyxovirus (e.g., influenza virus), rhabdovirus (e.g., rabies and vesicular stomatitis virus), paramyxovirus (e.g. measles and Sendai), positive-strand RNA viruses such as picornavirus and alphavirus, and double stranded DNA viruses including adenovirus, herpesvirus (e.g., herpes simplex virus types 1 and 2, Epstein-Barr virus, cytomegalovirus), and poxvirus (e.g. vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus, lentiviruses and baculoviruses.

In addition, an engineered viral vector can be used to deliver an isolated TLR4 activator or Cathepsin E inhibitor nucleic acid of the present invention. These vectors provide a means to introduce nucleic acids into cycling and quiescent cells, and have been modified to reduce cytotoxicity and to improve genetic stability. The preparation and use of engineered Herpes simplex virus type 1 (Krisky et al., 1997, Gene Therapy 4:1120-1125), adenoviral (Amalfitanl et al., 1998, Journal of Virology 72:926-933) attenuated lentiviral (Zufferey et al., 1997, Nature Biotechnology 15:871-875) and adenoviral/retroviral chimeric (Feng et al., 1997, Nature Biotechnology 15:866-870) vectors are known to the skilled artisan. In addition to delivery through the use of vectors, an isolated TLR4 activator or Cathepsin E inhibitor nucleic acid can be delivered to cells without vectors, e.g. as “naked” nucleic acid delivery using methods known to those of skill in the art. See, for example, U.S. Pat. Nos. 5,350,674 and 5,585,362.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York), and in Ausubel et al. (2001, Current Protocols in Molecular Biology, John Wiley & Sons, New York).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Various forms of an isolated TLR4 activator or Cathepsin E inhibitor nucleic acid, as described herein, can be administered or delivered to a mammalian cell (e.g., by virus, direct injection, or liposomes, or by any other suitable methods known in the art or later developed). The methods of delivery can be modified to target certain cells, and in particular, cell surface receptor molecules. As an example, the use of cationic lipids as a carrier for nucleic acid constructs provides an efficient means of delivering the isolated TLR agonist nucleic acid of the present invention.

Various formulations of cationic lipids have been used to deliver nucleic acids to cells (WO 91/17424; WO 91/16024; U.S. Pat. Nos. 4,897,355; 4,946,787; 5,049,386; and 5,208,036). Cationic lipids have also been used to introduce foreign polynucleotides into frog and rat cells in vivo (Holt et al., Neuron 4:203-214 (1990); Hazinski et al., Am. J. Respr. Cell. Mol. Biol. 4:206-209 (1991)). Therefore, cationic lipids may be used, generally, as pharmaceutical carriers to provide biologically active substances (for example, see WO 91/17424; WO 91/16024; and WO 93/03709). Thus, cationic liposomes can provide an efficient carrier for the introduction of polynucleotides into a cell.

Further, liposomes can be used as carriers to deliver a nucleic acid to a cell, tissue or organ. Liposomes comprising neutral or anionic lipids do not generally fuse with the target cell surface, but are taken up phagocytically, and the polynucleotides are subsequently subjected to the degradative enzymes of the lysosomal compartment (Straubinger et al., 1983, Methods Enzymol. 101:512-527; Mannino et al., 1988, Biotechniques 6:682-690). However, as demonstrated by the data disclosed herein, an isolated snRNA of the present invention is a stable nucleic acid, and thus, may not be susceptible to degradative enzymes. Further, despite the fact that the aqueous space of typical liposomes may be too small to accommodate large macromolecules, the isolated TLR4 activator or Cathepsin E inhibitor nucleic acid of the present invention is relatively small, and therefore, liposomes are a suitable delivery vehicle for the present invention. Methods of delivering a nucleic acid to a cell, tissue or organism, including liposome-mediated delivery, are known in the art and are described in, for example, Feigner (Gene Transfer and Expression Protocols Vol. 7, Murray, E. J. Ed., Humana Press, New Jersey, (1991)).

In other related aspects, the invention includes an isolated TLR4 activator or Cathepsin E inhibitor nucleic acid operably linked to a nucleic acid comprising a promoter/regulatory sequence such that the nucleic acid is preferably capable of delivering an isolated TLR4 activator or Cathepsin E inhibitor nucleic acid. Thus, the invention encompasses expression vectors and methods for the introduction of an isolated TLR4 activator or Cathepsin E inhibitor nucleic acid into or to cells.

Such delivery can be accomplished by generating a plasmid, viral, or other type of vector comprising an isolated TLR4 activator or Cathepsin E inhibitor nucleic acid operably linked to a promoter/regulatory sequence which serves to introduce the TLR4 activator or Cathepsin E inhibitor into cells in which the vector is introduced. Many promoter/regulatory sequences useful for the methods of the present invention are available in the art and include, but are not limited to, for example, the cytomegalovirus immediate early promoter enhancer sequence, the SV40 early promoter, as well as the Rous sarcoma virus promoter, and the like. Moreover, inducible and tissue specific expression of an isolated TLR4 activator or Cathepsin E inhibitor nucleic acid may be accomplished by placing an isolated TLR4 activator or Cathepsin E inhibitor nucleic acid, with or without a tag, under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In addition, promoters which are well known in the art which are induced in response to inducing agents such as metals, glucocorticoids, and the like, are also contemplated in the invention. Thus, it will be appreciated that the invention includes the use of any promoter/regulatory sequence, which is either known or unknown, and which is capable of driving expression of the desired protein operably linked thereto.

Selection of any particular plasmid vector or other vector is not a limiting factor in this invention and a wide plethora of vectors are well-known in the art. Further, it is well within the skill of the artisan to choose particular promoter/regulatory sequences and operably link those promoter/regulatory sequences to a DNA sequence encoding a desired polypeptide. Such technology is well known in the art and is described, for example, in Sambrook et al. (2001, Molecular Cloning: A Laboratory Manual, Cold Spring Harbor Laboratory, New York), and in Ausubel et al. (2001, Current Protocols in Molecular Biology, John Wiley & Sons, New York) and elsewhere herein.

### III. Pharmaceutical Compositions and Therapies

Administration of a TLR4 activator or Cathepsin E inhibitor comprising one or more peptides, small molecules, antisense nucleic acids, or antibodies of the invention in a method of treatment can be achieved in a number of different ways, using methods known in the art. Such methods include, but are not limited to, providing exogenous peptide inhibitor, small molecule, or an antibody to a subject or expressing a recombinant peptide inhibitor, small molecule, soluble receptor, or an antibody expression cassette.

In one embodiment, an exogenous TLR4 activator or Cathepsin E inhibitor peptide is administered to a subject. The exogenous peptide may also be a hybrid or fusion protein to facilitate, for instance, delivery to target cells or efficacy. In one embodiment, a hybrid protein may comprise a tissue-specific targeting sequence.

In another embodiment, an expression vector comprising an expression cassette encoding a TLR4 activator or Cathepsin E inhibitor protein, or fragment there of, or an antibody that will bind an epitope specific to Cathepsin E, or a fragment thereof, is administered to a subject. An expression cassette may comprise a constitutive or inducible promoter. Such promoters are well known in the art, as are means for genetic modification. Expression vectors and methods for the introduction of exogenous DNA into cells with concomitant expression of the exogenous DNA in the cells are described, for example, in Sambrook et al. (2001, Molecular Cloning: A Laboratory Manual, Cold Spring Harbor Laboratory Press, Cold Spring Harbor, N.Y.), and in Ausubel et al. (eds, 2005, Current Protocols in Molecular Biology, John Wiley & Sons, New York, N.Y.). In one embodiment, a cell comprising an expression vector of the invention is administered to a subject. Thus, the invention encompasses a cell comprising an isolated nucleic acid encoding a TLR4 activator or Cathepsin E inhibitor peptide, fusion protein or antibody of the invention.

Any expression vector compatible with the expression of a TLR4 activator or Cathepsin E inhibitor peptide, fusion protein, soluble receptor, or antibody of the invention is suitable for use in the instant invention, and can be selected from the group consisting of a plasmid DNA, a viral vector, and a mammalian vector. The expression vector, or a vector that is co-introduced with the expression vector, can further comprise a marker gene. Marker genes are useful, for instance, to monitor transfection efficiencies. Marker genes include: genes for selectable markers, including but not limited to, G418, hygromycin, and methotrexate, and genes for detectable markers, including, but not limited to, luciferase and GFP. The expression vector can further comprise an integration signal sequence which facilitates integration of the isolated polynucleotide into the genome of a target cell.

The therapeutic and prophylactic methods of the invention thus encompass the use of pharmaceutical compositions comprising TLR4 activator or Cathepsin E inhibitor peptide, fusion protein, small molecule, or antibody of the invention and/or an isolated nucleic acid encoding a TLR4 activator or Cathepsin E inhibitory peptide, fusion protein small molecule, or antibody of the invention to practice the methods of the invention. The pharmaceutical compositions useful for practicing the invention may be administered to deliver a dose of between 1 ng/kg/day and 100 mg/kg/day. In one embodiment, the invention envisions administration of a dose which results in a concentration of the compound of the present invention between 1 μM and 10 μM in a mammal.

Typically, dosages which may be administered in a method of the invention to an animal, preferably a human, range in amount from 0.5 μg to about 50 mg per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 μg to about 10 mg per kilogram of body weight of the animal. More preferably, the dosage will vary from about 3 μg to about 1 mg per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Other active agents useful in the treatment of fibrosis include anti-inflammatories, including corticosteroids, and immunosuppressants.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxy benzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, Pa.), which is incorporated herein by reference.

### Kits

The invention also includes a kit comprising a TLR4 activator, Cathepsin E inhibitor, or a combination thereof, of the invention and an instructional material which describes, for instance, administering the TLR4 activator, Cathepsin E inhibitor, or a combination thereof to a subject as a prophylactic or therapeutic treatment or a non-treatment use as described elsewhere herein. In an embodiment, this kit further comprises a (preferably sterile) pharmaceutically acceptable carrier suitable for dissolving or suspending the therapeutic composition, comprising a TLR4 activator, a Cathepsin E inhibitor, or a combination thereof of the invention, for instance, prior to administering the molecule to a subject. Optionally, the kit comprises an applicator for administering the inhibitor. In one embodiment of the invention, the applicator is designed for pulmonary administration of the TLR4 activator, Cathepsin E inhibitor, or combination thereof. In another embodiment, the kit comprises an antibody that specifically binds an epitope on TLR4 activator, Cathepsin E inhibitor, or a combination thereof. Preferably, the antibody recognizes a human Cathepsin E.

A kit providing a nucleic acid encoding a peptide or antibody of the invention and an instructional material is also provided.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The materials and methods employed in the experiments disclosed herein are now described.

### Generation of Transgenic Constructs and Mice:

### A. TLR4-Deficient

Tlr4^{-/-} and MyD88^{-/-} mice have been previously described (Zhang X., et al., 2005, J. Immunol. 175:4834-4838; Adachi O., et al., 1998, Immunity. 9:143-150; Hoshino K., et al., 1999, J. Immunol. 162:3749-3752). TLR4-deficient mouse lines C3H/HeJ and C57BL/10ScNJ and their respective controls were purchased from The Jackson Laboratory. C57BL/6J Tlr4^{-/-} mice were generated after greater than 10 generations of backcrossing onto a C57BL/6J background. WT littermates were used as controls. For antioxidant therapy, Tlr4^{-/-} mice were randomized into 3 groups at 3 weeks of age—NAC (1 g/kg body weight; Sigma-Aldrich) (apocynin (3 mg/kg body weight; Calbiochem; EMD Biosciences) (or vehicle-only drinking water—and then sacrificed at 3 months. Mice were maintained under specific pathogen-free conditions at the animal facility of Yale University School of Medicine. Animal protocols were reviewed and approved by the Animal Care and Use Committee at Yale University.

### B. CC10-Cathepsin E Transgenic Mice

CC10-Cathepsin E (CC10-Cathepsin E) transgenic mice were generated using human Cathepsin E (Cathepsin E) cDNA. A 1.207 kb Xba I and Spe I fragment containing the human Cathepsin E cDNA was amplified by PCR. This fragment was then ligated to INS-CC10-hGH (Dr. Paul Noble), which had been digested previously from pBS vector with Xba I and Spe I to create INS-CC10-Cathepsin E. The INS-CC10-Cathepsin E DNA fragment was then isolated by digestion with BssH II, purified, and used for microinjection. Tie2-rtTA were generated by ligating rtTA and poly A fragments (from pTet-ON, Clontech) into pSPtg.TsFXK (Dr. Flavell), which contains the Tie2 promoter/enhancer. The Tie2-rtTA DNA fragment was then isolated by digestion with Sal I, purified, and can be used for microinjection. Inducible, CC10-Cathepsin E transgenic mice were created using CC10-rtTA (Dr. Jack Elias) and a tetracyline-response element (TRE)-CatE construct which we generated by amplifying and digesting a 1.19 kb Sac II and Xba I fragment containing human CatE cDNA using Sac II and Xba I. The Cathepsin E cDNA was ligated to pTRE2 vector (BD Biosciences), which had been digested previously with Sac II and Xba I, to create TRE-Cathepsin E. The TRE-Cathepsin E DNA fragment was then isolated by digestion with Xho I and Ase I, purified, and used for microinjection performed by our Animal Genomics core.

Tail DNA was extracted and PCR performed using the following primers: INS-CC10-hCatE sense, 5′-ACACGCATACCCACACATAC-3′ (SEQ ID NO:3); antisense, 5′-TACTGGAGTCACTCCTCCCA-3′(SEQ ID NO:4). For Tie2-rtTA: sense, 5-GTCGCTAAAGAAGAAAGGGAAACAC-3 (SEQ ID NO:5); antisense, 5-TTCCAAGGGCATCGGTAAACATCTG-3 (SEQ ID NO:6). For TRE-Cre: sense, 5′-TGC CAC GAC CAA GTG ACA GCA ATG-3′ (SEQ ID NO:7); antisense, 5′-AGA GAC GGA AAT CCA TCG (SEQ ID NO:8). For Tie2-Cre: sense, 5′-CGA TGC AAC GAG TGA TGA GG-3′ (SEQ ID NO:9); antisense, 5′-CGC ATA ACC AGT GAA ACA GC-3′ (SEQ ID NO:10). For inducible CC 10-Cathepsin E tg mice: sense, 5-GTCGCTAAAGAAGAAAGGGAAACAC-3 (SEQ ID NO:11); antisense, 5-TTCCAAGGGCATCGGTAAACATCTG-3 (SEQ ID NO:12); hCathepsin E: sense, 5-TCCCCTCTGTGTACTGCACT-3 (SEQ ID NO:13); antisense, 5′-TACTGGAGTCACTCCTCCCA-3′ (SEQ ID NO:14). The following PCR protocol was used: 95° C. for 5 min; 30 cycles of 95° C. for 30 sec, 56° C. for 30 sec, and 72° C. for 45 sec; and a final extension at 72° C. for 10 min.

### Genotyping and Sequencing of Transgenic and Knockout Mice

Tail DNA was extracted and PCR performed using the following: INS-CC10-hCatE: sense, 5′-ACACGCATACCCACACATAC-3′(SEQ ID NO:3); antisense, 5′-TACTGGAGTCACTCCTCCCA-3′(SEQ ID NO:4). For Tie2-rtTA: sense, 5-GTCGCTAAAGAAGAAAGGGAAACAC-3(SEQ ID NO:5); antisense, 5-TTCCAAGGGCATCGGTAAACATCTG-3(SEQ ID NO:6). For TRE-Cre: sense, 5′-TGC CAC GAC CAA GTG ACA GCA ATG-3′(SEQ ID NO:7); antisense, 5′-AGA GAC GGA AAT CCA TCG (SEQ ID NO:8). For Tie2-Cre: sense, 5′-CGA TGC AAC GAG TGA TGA GG-3′ (SEQ ID NO:9); antisense, 5′-CGC ATA ACC AGT GAA ACA GC-3′ (SEQ ID NO:10).

For inducible CC10-Cat E tg mice: sense, 5-GTCGCTAAAGAAGAAAGGGAAACAC-3 (SEQ ID NO:11); antisense, 5-TTCCAAGGGCATCGGTAAACATCTG-3 (SEQ ID NO:12); hCat E: sense, 5-TCCCCTCTGTGTACTGCACT-3 (SEQ ID NO:17); antisense, 5′-TACTGGAGTCACTCCTCCCA-3′ (SEQ ID NO:18).

Nox3 null mice were subjected to gene sequencing using mouse tail DNA to amplify Nox3 by PCR. Nox3 primers were 5′-GCTAGCAGTGGATGGGCCC-3′ (SEQ ID NO:19) and 5′-ATCAGCCACTGAGTGGAGCT-3′ (SEQ ID NO:20).

The following PCR protocol was used: 95° C. for 5 min; 30 cycles of 95° C. for 30 sec, 56° C. for 30 sec, and 72° C. for 45 sec; and a final extension at 72° C. for 10 min. The fragment length is 525 bp. A 1% agarose gel was run, the DNA purified and the PCR fragment sequenced by using primer 5′-GCTAGCAGTGGATGGGCCC-3′ (SEQ ID NO:20). A T-to-A transversion was identified in the splice donor consensus sequence at the 3′ end of exon 10.

### Irradiation and Bone Marrow Transplantation

Whole-body irradiation of recipient mice and harvesting of donor bone marrow was performed as described previously (Cohn L. et al., 2001, J. Immunol. 166:2760-2767). Briefly, donor bone marrow was flushed from the femurs, tibias, and humeri of 6- to 8-week-old mice with cold sterile PBS. The cell suspension was repetitively aspirated until no visible fragments remained. Cells were pelleted at 300 g for 10 minutes at 4° C. before counting. Recipient mice at 3 weeks of age underwent whole-body irradiation (1,000 cGy) followed by intravenous injection of whole bone marrow cells (9×10⁶ cells in 0.2 ml PBS). After bone marrow transplantation, mice were maintained until 3 months of age under specific pathogen-free conditions at the Yale University School of Medicine animal facility and fed acidic water.

### Histology

Animals were anesthetized, the pulmonary intravascular space was cleared. Lungs sections were processed for H&E, orcein, TUNEL, and 8-OH-dG staining as described previously (Zhang X. et al., 2005, J. Immunol. 175:4834-4838). Immunohistochemistry was also applied to stain the elastin in the lungs. Briefly, formalin-fixed, paraffin-embedded lung tissue sections were deparaffinized with xylene, rehydrated gradually with graded alcohol solutions, and then washed with deionized water. Sections were incubated with a 1:200 dilution of anti-elastin monoclonal antibody (Chemicon International) at 37° C. for 2 hours. After PBS washes, sections were incubated with a 1:200 dilution of biotinylated anti-mouse IgG at 37° C. for 60 minutes, and peroxidase-conjugated streptavidin-biotin complex was incubated at 37° C. for 60 minutes. After further washing the sections with PBS, 3,3′-diaminobenzidine tetrachloride/nickel-cobalt substrate was applied as the chromogen, yielding a brown-black-colored reaction product, and counterstained the sections with methyl green (Vector Laboratories).

For Cathepsin E staining, formalin-fixed, paraffin-embedded lung tissue sections were deparaffinized, rehydrated, washed with deionized water, digested with trypsin, and incubated with 3% hydrogen peroxide. For detection of Cathepsin E, lung tissue sections were blocked with 10% normal goat serum in PBS for 30 minutes. Sections were incubated 1 h at 37° C. with a rabbit polyclonal anti-CatE antibody (Novus Biologicals), washed with PBS, and incubated at room temperature for 45 minutes with PowerVision poly-HRP anti-rabbit antibody (ImmunoVision Technologies). After further washing the sections with PBS, diaminobenzidine substrate was applied as the chromogen, giving a brown reaction product, and counterstained the sections with Mayer's hematoxylin (Zymed). The rabbit IgG instead of the Cathepsin E antibody was used for negative control.

### Immunofluorescence Studies

Formalin-fixed, paraffin-embedded lung tissue sections were deparaffinized, rehydrated, and washed with deionized water. Antigen retrieval was performed by placing the slides in BD Retrivagen A solution (BD Biosciences) at 89° C. for 10 minutes and cooled slowly to room temperature. After blocking with 5% normal donkey serum for 30 min at room temperature, the slides were incubated with primary antibody at 4° C. overnight. For Nox3, lung sections were double labeled with rabbit anti-Nox3 (1:300; Santa Cruz) and goat anti-CC10 (1:500) or goat anti-Sp-C (1:500) or goat anti-CD31 (1:300) with dilutions made in serum-blocking buffer. Lenti-GFP was detected in lungs using rabbit anti-GFP (1:500; Invitrogen) and the cell-specific antibodies listed above. Donkey anti-rabbit-IgG Cy3 and donkey anti-goat- or anti-rabbit IgG FITC were used as secondary antibodies, depending on the origin of the primary antibody. Cat E expression was detected using rabbit anti-Cat E (1:300; Santa Cruz) and goat anti-Sp-C (1:500) or biotin-labeled rat anti-F4/80 (1:400; BD). Secondary antibodies were donkey anti-rabbit-IgG Cy3 and donkey anti-goat-IgG FITC (Sp-C) or donkey SA Alexa 555 (for F4/80). All secondary antibodies were purchased from Jackson ImmunoResearch Labs. Negative control sections were processed in the same way without primary antibody. The sections were washed in PBS and immunoreactive cells were identified after 45 min incubation at room temperature with secondary antibody. The sections were washed with PBS, covered with Prolong Gold Antifade Reagents (Molecular Probes) and examined under fluorescence microscopy using the appropriate excitation wavelength.

### Lung Volume, Morphometric Assessment, and Chord Length Measurements

Mice were anesthetized, the trachea was cannulated, and the lungs were removed and inflated with PBS at 25 cm H₂O. The size of the lung was evaluated by volume displacement. Hematoxylin and eosin (H&E) or periodic acid-Schiff with diastase (PAS) staining was performed after pressure fixation with Streck solution (Streck Laboratories) in the Research Pathology Laboratory at Yale University. Alveolar size was estimated from the mean chord length of the airspaces.

### Total Lung Cell and Inflammatory Cell Isolation.

For total lung cell isolation, mice were anesthetized, and the pulmonary vasculature was perfused with PBS until free of blood. The lungs were filled with 1-2 ml of dispase II (Roche Diagnostics) and then placed into a conical tube containing 2 ml of dispase II solution at room temperature for 45 minutes. The lungs were then transferred to a petri dish containing PBS with 50 U/ml DNase I (Sigma-Aldrich), 5% FBS, and 1 mg/ml collagenase A (Roche Diagnostics). After separating the digested tissue, lungs were incubated for 60 minutes at 37° C., and the resulting cell suspensions were filtered through 100-μm cell strainers (BD Falcon; BD Biosciences). The total lung inflammatory cell isolation has been described previously (Cohn L., et al., 2001, J. Immunol. 166:2760-2767).

### Isolation of Primary MLECs.

MLECs were isolated by as described by Kuhlencordt et al. (Kuhlencordt P. J., et al., 2004, Am. J. Physiol. Cell Physiol. 286:C1195C1202.), with some modifications. Briefly, lungs were extracted, minced, and digested for 1 hour at 37° C. with 0.1% collagenase (Roche Diagnostics) in RPMI-1640 with 100 U/ml penicillin G and 100 μg/ml streptomycin. The digest was passed through a 100-μm cell strainer to remove undigested tissue fragments. Cells were pelleted at 200 g for 5 minutes; resuspended in MLEC medium containing 20% FBS, 40% DMEM, and 40% F12 with 100 U/ml penicillin G and 100 μg/ml streptomycin; and plated onto 0.1% gelatin-coated T75 flasks. Cells were washed after 24 hours and cultured for 2-4 days. Cells were trypsinized with 2 ml trypsin/EDTA, PBS added, and spun for 5 minutes at 200 g to remove the supernatant. Cells were resuspended in 2% FBS containing 10 μl biotin-labeled rat anti-mouse CD31 (PECAM-1) antibody (BD Biosciences—Pharmingen). After incubation on ice for 30 minutes, the cells were washed with streptavidin magnetic beads (New England Biolabs Inc.). Cells were washed with 2% FBS, resuspended in 5 ml of 2% FBS, and incubated on ice for 30 minutes. The cells were then placed on the magnet for 5 minutes, and unbound cells were removed while bound cells were resuspended in medium and plated onto a 0.1% gelatin-coated T25 flask. CD31 staining and flow cytometry confirmed that greater than 95% of the cells were endothelial cells. Cells were maintained in 40% DMEM and 40% F12 tissue culture medium supplemented with 20% FBS.

### Flow Cytometric Analysis for TUNEL and Annexin V-PI Staining in Lung Cells.

Total lung cells of Tlr4^{-/-} and WT mice were resuspended in PBS and adjusted to 2×10⁷ cells/ml. After fixation with 2% paraformaldehyde and permeabilization with 0.1% Triton X-100, the lung cells were incubated with TUNEL reaction mixture for 60 minutes. Negative control was incubated with TUNEL reaction buffer without terminal deoxynucleotidyl transferase. Cells were washed twice with PBS, and TUNEL staining was detected by flow cytometry (BD) and then analyzed using CellQuest FACSConvert software (version 7.5.3; BD). For annexin V-PI staining, 1×10⁶ cells were resuspended in 1× binding buffer (10 mM HEPES/NaOH, pH 7.4; 140 mM NaCl; and 2.5 mM CaCl₂), 5 μl annexin V and 5 μl PI were added, and cells were incubated for 15 minutes in the dark. Binding buffer (400 μl) was then added to each tube and analyzed by flow cytometry (BD).

### Protein Analysis

Western Blots and BAL ELISAs as described in Lee et al., 2006, J. Clin. Invest. 116:163-173, incorporated herein in its entirety. Cathepsin E (Cathepsin E) in the BAL and serum was assayed by sandwich ELISA. The capture antibody was anti-mouse or anti-human Cathepsin E antibody (R&D Systems). Biotinylated anti-mouse or anti-human Cathepsin E antibody (R&D Systems) and Streptavidin-HRP (Invitrogen) were used for detection. Recombinant mouse Cathepsin E or human Cathepsin E (R&D Systems) was used to create the standard curve. Briefly, 96-well microplates were coated with the capture antibody (1.5 mg/ml in PBS) at room temperature overnight. Plates were then washed with wash buffer (0.05% Tween 20 in PBS) and blocked with 1% bovine serum albumin (BSA) for 1 h. BAL samples (100 μl, diluted in PBS) were incubated at room temperature for 2 h. Plates were washed with wash buffer, the biotinylated anti-mouse Cathepsin E antibody (0.1 μg/ml in PBS) was added, and plates were incubated at room temperature for 2 h. The bound antibodies were detected via sequential incubation with Streptavidin-HRP (100 μl, 1:3000 dilution in PBS) for 30 min and the enzyme substrate TMB solution (50 ul). The reaction was stopped by the addition of 6N HCl (50 ml/well). Plates were read on an ELISA reader at 450 nm.

### Caspase 3 Expression by Flow Cytometry

Cells were centrifuged and fixed with 2% formaldehyde for 10 minutes at 37° C. Tubes were chilled on ice for 1 minute, and cells were permeabilized by adding ice-cold 100% methanol slowly, while gently vortexing, so that the final concentration was 90% methanol. After incubation on ice for 30 minutes, cells were counted using a hemacytometer, and 1×10⁶ cells were aliquoted into each assay tube. Incubation buffer (0.5% BSA in 1×PBS; 2 ml) was added to each tube and rinsed by centrifugation. Cells were resuspended in 100 μl incubation buffer per assay tube and allowed to sit at room temperature for 10 minutes. Cleaved caspase 3 rabbit monoclonal antibody was added to the assay tubes at 1:100 dilution and incubated for 60 minutes at room temperature. Cells were rinsed in incubation buffer by centrifugation, resuspended in Alexa Fluor 555 F(ab′)2 fragment of goat anti-rabbit IgG (H+L) antibody (1:1,000 dilution), and incubated for 30 minutes at room temperature. After an additional rinse in incubation buffer by centrifugation, the cells were resuspended in 0.5 ml PBS and analyzed by flow cytometry (BD).

Preparation of siRNA and Transfection of siRNA Duplexes

Nox3 and TLR4 siRNA was purchased from Santa Cruz Biotechnology Inc. Nonspecific siRNA scrambled duplex probes (sense, 5′-GCGCGCUUUGUAGGAUUCG-3′ (SEQ ID NO:21); antisense, 5′-CGAAUCCUACAAAGCGCGC-3′ (SEQ ID NO:22)) were synthesized by Dharmacon Research Inc. as previously described (Zhang X., et al., 2004, J. Biol. Chem. 279:10677-10684.). MLECs were seeded onto 6-well plates 1 day prior to transfection using 40% DMEM and 40% F12 tissue culture medium supplemented with 20% FBS, without antibiotics. At the time of transfection with the specific siRNA, the cells were 50%-60% confluent. Oligofectamine Reagent (Invitrogen) was used as the transfection agent, and cells were then incubated for 6 hours, after which FBS was added to reach a final concentration of 20% FBS in the wells. After 48 hours' incubation, the cells were collected and subjected to the assays described below.

RNA Extraction and mRNA Analyses

Total RNA from lung tissue was extracted using TRI_{ZOL} reagent (Invitrogen) according to the manufacturer's instructions. The following primers were used in RT-PCR: mouse caspase 3,5′-TGTCATCTCGCTCTGGTACG-3′ (SEQ ID NO:23) and 5′-TAACGCGAGTGAGAATGTGC-3′ (SEQ ID NO:24); Nox-3,5′-GCCTACGGGATAGCTGTCAA-3′ (SEQ ID NO:25) and 5′-GGACTGCAGATGGGTGACTC-3′ (SEQ ID NO:26); β-actin, 5′-GTGGGCCGCTCTAGGCACCAA-3′ (SEQ ID NO:27) and 5′-CTCTTTGATGTCACGCACGATTTC-3′ (SEQ ID NO:28). Primers for MMPs, Cathepsins, and TIMPs were described previously (Zheng T., et al., 2000, J. Clin. Invest. 106:1081-1093). RT-PCR was performed using RT-PCR Master Mix (USB). Conditions for RT-PCR were as follows: 1 cycle at 42° C. for 30 minutes; 1 cycle at 95° C. for 3 minutes; 30 cycles at 95° C. for 30 seconds, 60° C. for 1 minute, and 68° C. for 90 seconds; and 1 cycle at 68° C. for 5 minutes. Each reaction product (10 μl) was then separated on a 1% agarose gel containing 0.5 μg/ml ethidium bromide. Real-time RT-PCR was performed using a QuantiTect SYBR Green RT-PCR kit (QIAGEN) according to the manufacturer's instructions. Reactions were made by a combination of 12.5 μl SYBR RT-PCR Master Mix (QIAGEN), 0.25 μl QuantiTect RT Mix (QIAGEN), 1 μl upstream primer, 1 μl downstream primer, 8.75 μl RNase-free water, and 1.5 μl (200 ng/μl) RNA template. A negative control containing no RNA template was introduced in each run. Mouse Gapdh gene was amplified as an internal control. RT-PCR was performed using ABI Sequence Detection System (Applied Biosystems), in which the mixture was heated to 50° C. for 30 minutes for reverse transcription and 95° C. for 15 minutes, then cycled 40 times at 94° C. for 15 seconds, 60° C. for 30 seconds, and 72° C. for 30 seconds. The primers used are depicted in Table I.

TABLE 1

| | SEQ | | SEQ | |
|---|---|---|---|---|
| | ID | | ID | |
| Target | NO. | Primer 1 (5′-3′) | NO. | Primer 2 (5′-3′) |
| Cathepsin B | 29 | GCAGGACTTCCAAAAGAACGA | 30 | GACGAATGCCTGCCACAAG |
| Cathepsin S | 31 | GAGACCCTACCCTGGACTACCA | 32 | CCCAGATGAGACGCCGTACT |
| Cathepsin D | 33 | CCCCTCCATTCATTGCAAGAT | 34 | CAAAGGACGTGCCGTTCTTC |
| Cathepsin H | 35 | GATTGTGCCCAAGCCTTCAA | 36 | CTGTCTTCTTCCATGATGCCC |
| MMP-12 | 37 | CATTTCGCCTCTCTGCTGATG | 38 | TTGATGGTGGACTGCTAGGTTTT |
| MM13-9, | 39 | CCCTGGAACTCACACGACA | 40 | GGAAACTCACACGCCAGAAG |
| MMP-13 | 41 | TCCCTGGAATTGGCAACAAAG | 42 | GGAATTTGTTGGCATGACTCTCAC |
| MMP-14 | 43 | AAGGCTGATTTGGCAACCAT | 44 | AGCGCTTCCTCCGAACATT |
| Nox1 | 45 | CCTTCCATAAGCTGGTGGCA | 46 | GGCCTGTTGGCTTCTTCTGTAG |
| Nox2 | 47 | CTTATCACAGCCACAAGCATTGAA | 48 | CACCCATTCACACTGACCTCTG |
| Nox3 | 49 | CTCGTTGCCTACGGGATAGC | 50 | CCTTCAGCATCCTTGGCCT |
| Nox4 | 51 | ACAACCAAGGGCCAGAATACTACTAC | 52 | GGATGAGGCTGCAGTTGAGG |
| TLR4 | 53 | TCTGATCATGGCACTGTTCTTCTC | 54 | GGGATTCAAGCTTCCTGGTGT |
| IL-1β | 55 | CTGAACTCAACTGTGAAATGCCA | 56 | AAAGGTTTGGAAGCAGCCCT |
| TNF-α | 57 | AGTTCCCAAATGGCCTCCC | 58 | GCTACGACGTGGGCTACAGG |
| IL-6 | 59 | AGAAAACAATCTGAAACTTCCAGAGAT | 60 | GAAGACCAGAGGAAATTTTCAATAGG |

The relative quantification values for these gene expressions were calculated from the accurate threshold cycle (C_{T}), which is the PCR cycle at which an increase in reporter fluorescence from SYBR green dye can first be detected above a baseline signal. The C_{T} values for GAPDH were subtracted from the C_{T} values for MMP-2, -9, -12, -13, and -14; Cathepsin B, S, D, and H; and TLR4 in each well to calculate ΔC_{T}. The triplicate ΔC_{T} values for each sample were averaged. To calculate the fold induction of MMPs, Cathepsins, Noxs, TLR4, IL-1β, TNF-α, and IL-6 mRNA over controls (ΔΔC_{T}), the average ΔC_{T} values calculated for WT animals were subtracted from ΔC_{T} values calculated for Tlr4^{-/-} animals. Next, the fold induction for each well was calculated using the 2^{-(ΔΔCT)} formula (Lee P. J., et al., 2006, J. Clin. Invest. 116:163-173.). The fold induction values for triplicate wells were averaged, and data were presented as the mean±SEM of triplicate wells.

### Elastolytic Activity

The elastolytic activities in lung tissues, BAL, and cell lysates was determined by hydrolysis of insoluble elastin and detected spectrophotometrically using elastin-Congo red as a substrate according to the method described previously (Rust L., et al., 1994, Methods Enzymol. 235:554-562). Briefly, elastin-Congo red (Sigma-Aldrich) was suspended in 0.1 M Tris-HCl buffer (pH 7.4) at a concentration of 10 mg/ml. A total 200 μl of reaction mixture consisted of elastin-Congo red suspension (final concentration, 5 mg/ml), varying concentrations of porcine elastase (Sigma-Aldrich) to generate a standard curve, and varying amounts of the sample of interest diluted in the same buffer. The mixture was incubated for 24 hours at 37° C., and the reaction mixture was filtered with a 40-μm mesh and then centrifuged for 5 minutes at 850 g at room temperature. The optical density of the supernatant was measured at 485 nm with a DYNEX Revelation version 3.2 spectrophotometer. The elastolytic activity was calculated as ng elastase/μg protein in the lysates according to the standard curve established by the reaction of porcine elastase and elastin-Congo red.

### Assessment of Antiprotease Activity

EIC was assessed in BAL and serum using previously described methods (Klumpp T. et al., 1979, Clin. Chem. 25:969-97; Cavarra E., et al., 2001, Am. J. Respir. Crit. Care Med. 164:886-890). Effects of cigarette smoke in mice with different levels of alpha(I)-prot). Briefly, BAL and serum were tested for EIC against porcine pancreatic elastase (PPE). EICs against PPE (type III; Sigma-Aldrich) in BAL and serum were determined using succinyl-trialanine-p-nitroanilide (SAPNA; Sigma-Aldrich) as the elastase substrate. EIC was expressed as percentage of enzyme activity inhibited by serum or BAL samples. Serum or BAL samples were incubated with PPE at room temperature for 20 minutes before adding the substrate SAPNA. Substrate hydrolysis was checked spectrophotometrically for 3 minutes at 410 nm. The percentage of inhibition was calculated as follows: [(uninhibited rate−inhibited rate)/uninhibited rate]×100, where the uninhibited rate reflects the rate at which the PPE degrades its substrate, SAPNA, and the inhibited rate reflects the rate at which the PPE degrades SAPNA in the presence of serum or BAL.

### Total Antioxidant Capacity

Total antioxidant capacity was measured in cell-free BAL and serum using the Quantitative Assay for Total Antioxidant Potential kit (Oxis International Inc.) according to the manufacturer's instructions. Briefly, equal amounts of BAL or serum were incubated with Bathocuproine (2,9-dimethyl-4,7-diphenyl-1,10-phenanthroline) and Cu²⁺-containing reagents. Bathocuproine selectively forms a 2:1 complex with Cu⁺, which has a maximum absorbance at 490 nm. A standard of a known uric acid concentration was used to create a calibration curve. Results are expressed as μM copper-reducing equivalents.

### Reduced and Oxidized GSH Content

The ratio of reduced GSH to oxidized GSH was measured in cell-free BAL using GSH/GSSG-412 kit (Oxis International Inc.) according to the manufacturer's instructions.

### Lipid Peroxidation

Malondialdehyde and 4-hydroxyalkenals were measured using Lipid Peroxidation Assay kit (Calbiochem; EMD Biosciences) according to the manufacturer's instructions.

### Cathepsin E Detection in Urine

Cathepsin E in the urine was assayed by sandwich ELISA. The capture antibody was an anti-mouse Cathepsin E antibody obtained from R&D systems (Minneapolis, Minn.). Biotinylated anti-mouse Cathepsin E antibody (R&D Systems)) and Streptavidin-HRP (Invitrogen) were used for detection in this assay and recombinant mouse Cathepsin E (R&D Systems) was used to create the standard curve. Briefly, 96-well microplates were coated with the capture antibody (1.5 g/ml in PBS) at room temperature overnight. Plate were then washed with wash buffer (0.05% Tween 20 in PBS) and blocked with 1% bovine serum albumin (BSA) for 1 h. Urine samples (1000, diluted in PBS) was added and plates were incubated at room temperature for 2 h. The bound antibodies were detected via sequential incubation with Streptavidin-HRP (100 μl 1:3000 dilution in PBS) for 30 min and the enzyme substrate TMB solution (100 ul). Reaction was stopped by addition of 6N HCl (50 μl/well). Plates were read on an ELISA reader at 450 nm.

### Cathepsin E Activity

Cathepsin E activity in lung lysates using acid or neutral pH conditions was determined using a fluorogenic peptide substrate for Cathepsin E, MOCAc-Gly-Ser-Pro-Ala-Phe-Leu-Ala-Lys (dnp)-D-Arg-NH2 (Peptide Institute, Inc.). Briefly, lung tissue was homogenized in 50 mM NaOAc, 0.1M Nacl (pH 3.5) buffer or PBS (pH 7.4) buffer. A total 120 ml of reaction mixture consisted of lung lysates in different pH conditions (pH 3.5 or 7.4), sample buffers (pH 3.5 or 7.4), and MOCAc-Gly-Ser-Pro-Ala-Phe-Leu-Ala-Lys (dnp)-D-Arg-NH2 substrate. The mixture was incubated for 30 min at room temperature. Recombinant mouse Cathepsin E is used to generate a standard curve. The relative fluorescence unit of the supernatant was measured at excitation and emission wavelengths, 320 nm and 400 nm, respectively. Cathepsin E activity was calculated as ng Cathepsin E protein/mg lung protein in the lysates according to the standard curve established by the reaction of recombinant mouse Cathepsin E and MOCAc-Gly-Ser-Pro-Ala-Phe-Leu-Ala-Lys (dnp)-D-Arg-NH2 substrate.

### Carboxypeptidase A Activity

Carboxypeptidase A activity in lung lysates was determined by its ability to cleave a fluorescent peptide substrate, (7-Methoxycoumarin-4-yl) acetyl-Arg-Pro-Pro-Gly-Phe-Ser-Ala-Phe-Lys (2,4-Dinitrophenyl)-OH(R&D Systems). Briefly, lung tissue was homogenized in 25 mM 2-(Nmorpholino) ethanesulfonic acid (MES) and 5 mM dithiothreitol (DTT) (pH 5.5) buffer. A total 100 ml of reaction mixture consisted of equal amounts of lung lysate, sample buffer (pH 5.5), and (7-Methoxycoumarin-4-yl) acetyl-Arg-Pro-Pro-Gly-Phe-Ser-Ala-Phe-Lys (2,4-Dinitrophenyl)-OH substrate. The mixture was incubated for 60 min at room temperature. The relative fluorescence unit (RFU) of the supernatant was measured at excitation and emission wavelength at 320 nm and 400 nm, respectively.

### Cell Surface HA Expression

As described previously (7), a biotinylated HA binding protein (12.5 mg/ml) was added to 5×105 cultured lung epithelial cells and incubated at 4° C. for 30 min. The cells were then washed and incubated with PerCP-labeled anti-streptavidin for another 20 min. Cells were washed and then subjected to flow cytometry analysis.

Construction of Lentiviral shRNA Vectors

A lenti-Cat E-shRNA using two oligos, 5′GATCCCATACACATTACAGGACTCGTTGATATCCGCGAGTCCTGTAATG TGTATTTTTTTCCAAC3′ (SEQ ID NO. 61) and 5′TCGAGTTGGAAAAAA ATACACATTACAGGACTCG CGGATATCAA CGAGTCCTGTAATGTGTATGG3′ (SEQ ID NO. 62) (Siner et al., 2007, FASEB J. 21:1422-1432), which were annealed and ligated into pRNAT-U6.1/Lenti. The luciferase shRNA gene was inserted into the same lentiviral plasmid to generate a control vector (Lenti-Luc shRNA). These shRNA expression vectors are driven by mouse U6 promoter on pRNAT-U6.1/Lenti backbone plasmid and terminated by six T following the shRNA sequence. Lenti-Nox3-shRNA was constructed using the target sequence 5′-GCACCTTTGATATGGGCACA-3′ (SEQ ID NO. 63) from the mouse Nox3 gene. Two oligos, 5′-GATCCCGCACCTTTGATATGGGCACAACTCGAGTTGTGC CCATATCAAAGGTGCTTTTTCCAAC-3′ (SEQ ID NO. 64) and 5′-TCGAGTTGGAAAAAGCACCTTTGATATGGGCACAACTCGAGTTGTGCCCA TATCAAAGGTGCGG-3′ (SEQ ID NO. 65), were cloned into pRNAT-U6.1/Lenti. The vectors were then placed into ViraPower lentiviral expression system (Invitrogen). The vector plasmids and ViraPower Package mixture were co-transfected into 293T cells.

### Construction of Lentiviral Overexpression Vectors

The lentiviral protein overexpression vector backbone (pFUW self-inactivating vector) and lentiviral GFP vector pFUGW self-inactivating vector were kindly provided by Dr. David Baltimore (California Institute of Technology). These two vectors contain the same human polyubiquitin C promoter, which produces high and reliable expression. For lentiviral Nox3, Nox3 cDNA from ATG to the stop codon was amplified by PCR and ligated into Hap I and Asc I sites on pFUW vector. Lentiviral GFP or lentiviral Nox3 was produced by transfecting the transfer vector pFUGW or pFUW-Nox3, the HIV-1 packaging vector delta 8.9, and the VSVG envelope glycoprotein into 293T fibroblasts.

### Lentiviral Concentration, Titration, and Intranasal Administration

Forty-eight hours after transfection of 293T cells with the lentiviral vector, viral particles in the supernatants were harvested and concentrated by ultracentrifugation at 25,000 rpm for 90 at 4° C. The resulting lentiviral pellet is dissolved into cold PBS (with Ca and Mg). Lentiviral titration is performed by using QuickTite Lentiviral Quantitation Kit (Cell Biolabs). The final titer of concentrated lentivirus is normalized to 1.5×1012VP/ml in PBS for intranasal administration. Each mouse is given 6×109 VP of the control lentivirus or targeting lentivirus intranasally.

### Statistics

Data are expressed as mean±SEM and were analyzed by 2-tailed Student's t test. P values less than 0.05 were considered to be significant.

The results of the experiments presented in this Example are now described.

### Experimental Example 1

### TLR4 and MyD88 Deficiencies Result in Emphysema

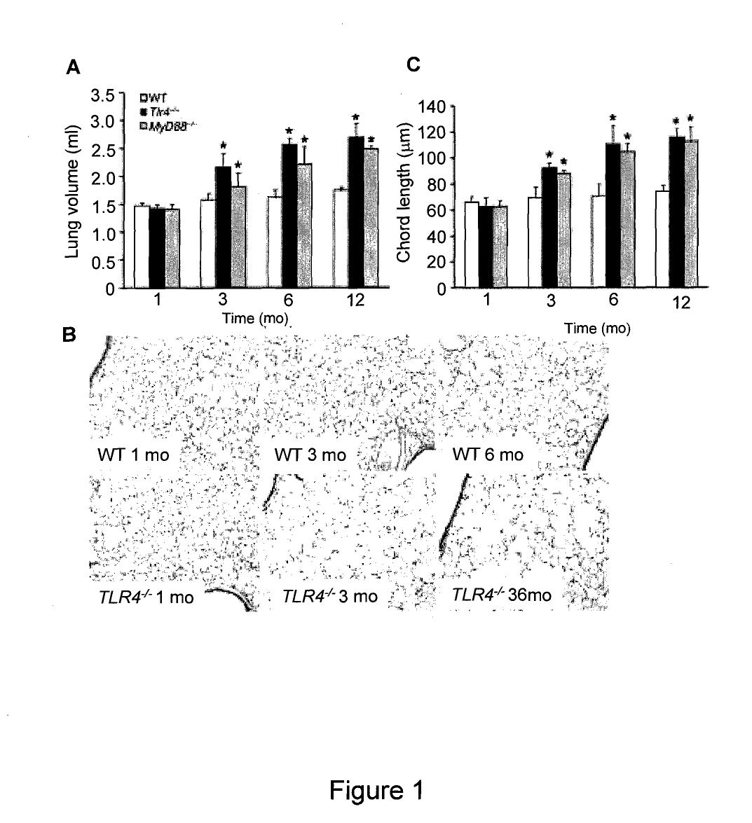

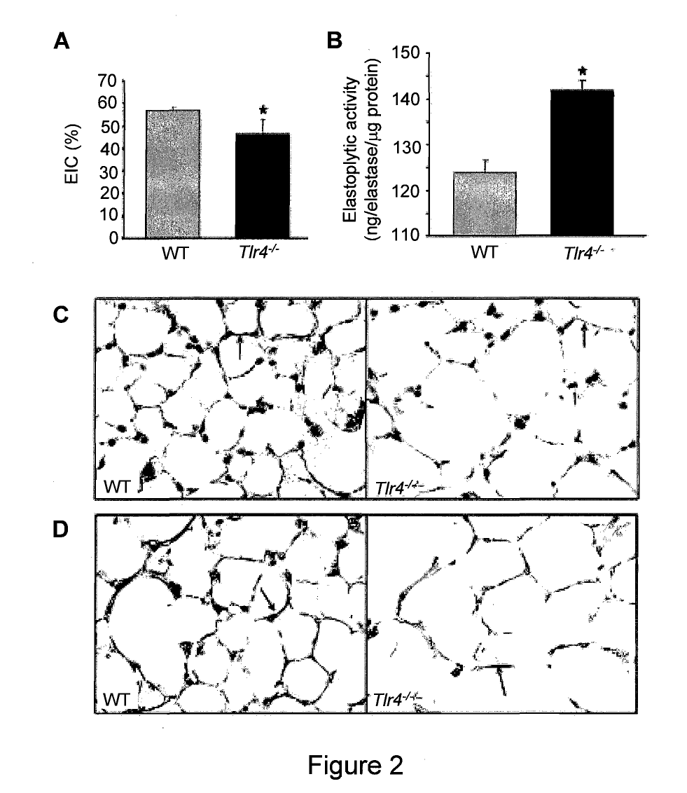

The role of TLR4 in conditions of ambient oxygen were investigated by examining the lungs of Tlr4^{-/-} mice in an unchallenged state. There were no differences between the lungs of WT and Tlr4^{-/-} mice at 1 and 2 months of age, but by 3 months Tlr4^{-/-} mice showed significantly increased lung volumes (FIG. 1A). Emphysema progressed and appeared to peak between 6 months and 1 year of age. There were no differences between the body weights of WT and Tlr4^{-/-} mice at 1, 3, 6, and 12 months of age. Histologic evaluation revealed enlargement of the air spaces distal to the terminal bronchioles accompanied by destruction of the normal alveolar architecture in Tlr4^{-/-} mice, characteristic of emphysema (FIG. 1B). Morphometric quantitation of the airspace enlargement revealed increased chord lengths in Tlr4^{-/-} mice (FIG. 1C). Using 3 months of age as a representative time point, data were confirmed in different strains of TLR4-deficient mice, indicating that the observed phenotype was not strain dependent. C3H/HeJ, C57BL/10ScNJ, and C57BL/6J Tlr4^{-/-} mice all exhibited increased lung volumes (Supplemental FIGS. 2-4). Mice deficient in MyD88, a central TLR adaptor protein, were also examined. MyD88^{-/-} mice showed progressive emphysema as well, suggesting that a Myd88-dependent TLR4 pathway is responsible for TLR4-mediated maintenance of lung integrity (FIG. 1, A and B).

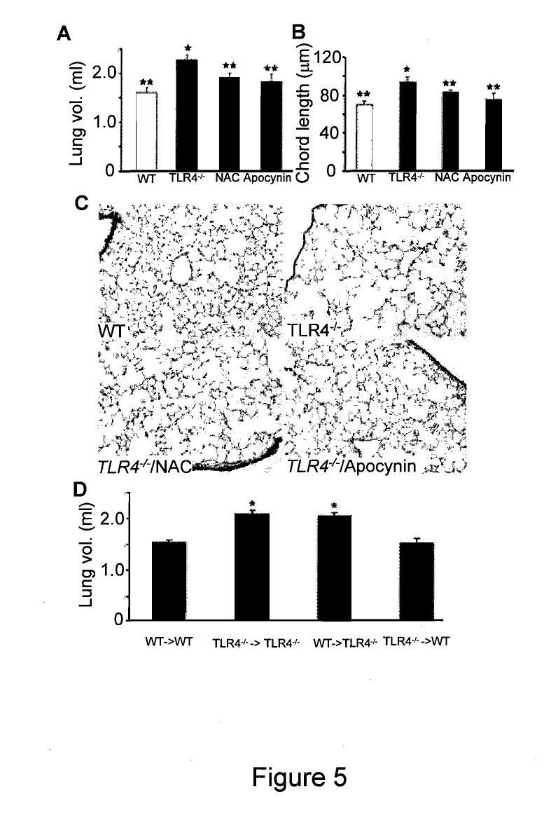

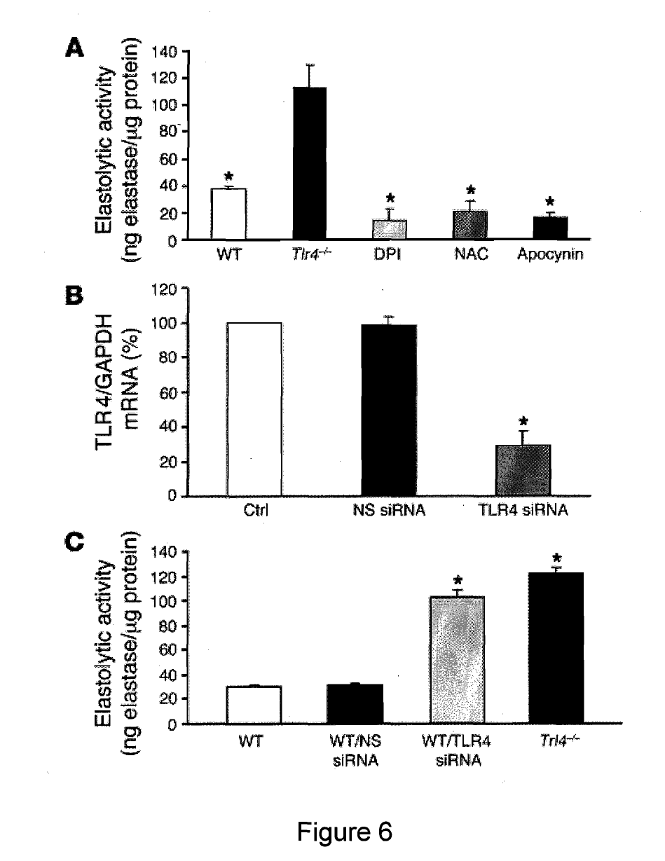

Increased lung inflammation and the overexpression of soluble mediators such as IL-13, IL-1β, IFN-γ, and VEGF are important in the pathogenesis of human as well as experimental emphysema (14. Lappalainen U., et al., 2005, Am. J. Respir. Cell Mol. Biol. 32:311-318; Lee C. G., et al., 2004, Nat. Med. 10:1095-1103; Zheng T., et al. 2000, J. Clin. Invest. 106:1081-1093; Zheng T., et al., 2005, J. Immunol. 174:8106-8115). Therefore, the question of whether Tlr4^{-/-} mice exhibited increased lung inflammation and increased cytokines and growth factors was investigated. Interestingly, there were no significant differences in the number of inflammatory cells isolated from bronchoalveolar lavage fluid (BAL) or from total lung digests of Tlr4^{-/-} and WT mice (Supplemental FIG. 5). In addition, Tlr4^{-/-} mouse lungs did not exhibit increased IL-1β, TNF-α, IL-6, VEGF, IL-13 and IFN-γ mRNA or protein compared with WT lungs (Supplemental FIG. 6). These data indicated that the etiology of emphysema in Tlr4^{-/-} mice could not be attributed to increased inflammation or cytokine and growth factor expression.

### Experimental Example 2

### TLR4-Deficient Mice Show Decreased Elastase Inhibitory Capacity

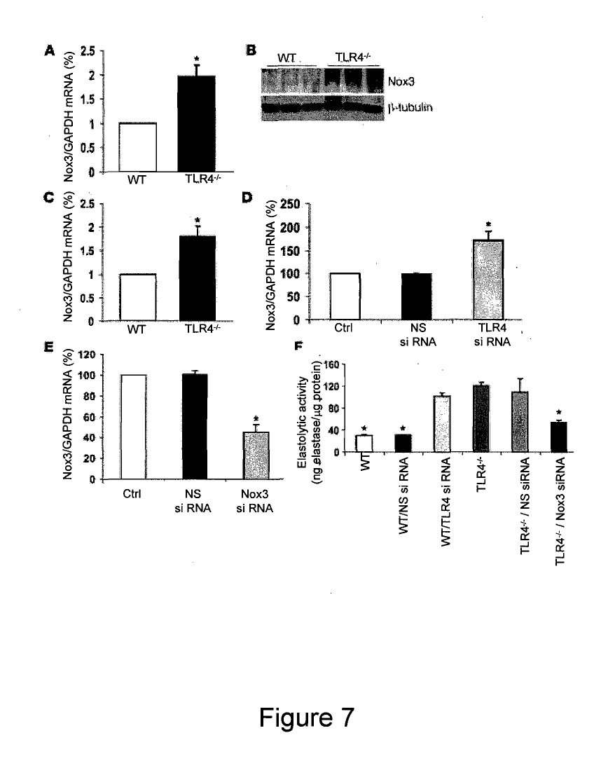

A protease/antiprotease imbalance leading to the breakdown of lung elastin is thought to play a key role in the pathogenesis of pulmonary emphysema. Therefore, antiprotease activity was quantitated by measuring the elastase inhibitory activity in BAL and serum of Tlr4^{-/-} and WT mice. Tlr4^{-/-} mice showed a marked decrease in their elastase inhibitory capacity (EIC) in BAL (FIG. 2A), as well as in serum (data not shown), compared with WT mice. The presence of elastolytic activity in the lung lysates of Tlr4^{-/-} mice was also assayed. Markedly increased elastolytic activity was found in Tlr4^{-/-} mice compared with WT mice (FIG. 2B). Similar results were also found in BAL (data not shown). The relationship between decreased elastase inhibitory activity and increased elastolytic activity was examined in the lungs of Tlr4^{-/-} mice to determine if decreased elastase inhibitory activity led to the destruction of elastin-containing elastic fibers in the lung parenchyma with subsequent increase in lung compliance, a hallmark of human emphysema (Barnes P. J., et al., 2003, Eur. Respir. J. 22:672-688). Histochemical analyses for elastin in the elastic fibers as assessed by orcein and elastin staining showed thin, fragmented elastin segments in Tlr4^{-/-} mice, whereas the elastic fibers in WT mice appeared intact (FIG. 2, C and D). Pulmonary function testing also revealed increased lung compliance in Tlr4^{-/-} mice compared with WT mice (Supplemental FIG. 7). α1-AT is the prototypic member of the serine protease inhibitor (serpin) superfamily of proteins, which play a major role in inactivating neutrophil elastase and other proteases and thus maintaining the protease-antiprotease balance in the lung. Therefore, α1-AT concentrations were measured in the serum and BAL of Tlr4^{-/-} and WT mice but no differences were found.

There is accumulating evidence for the role of MMPs and Cathepsins in the pathogenesis of pulmonary emphysema (Zheng T., et al., 2005, J. Immunol. 174:8106-8115; Shapiro S. D., 2002, Biochem. Soc. Trans. 30:98-102; Elkington P. T., et al., 2006, Thorax. 261:259-266). Patients with COPD exhibit increased concentrations of MMP-9 (gelatinase B) and MMP-12 in BAL and sputum, respectively (Finlay G. A., et al., 1997, Am. J. Respir. Crit. Care Med. 156:240-247; Demedts I. K., et al., 2006, Thorax. 61:196-201). Animal models have shown that cigarette smoke-induced emphysema does not occur in mice lacking MMP-12 (Hautamaki R. D., et al., 1997, Science 277:2002-2004). However, when the mRNA and protein levels of key MMPs, TIMPs, and Cathepsins associated with the development of emphysema were examined in Tlr4^{-/-} and WT mouse lungs, no significant differences were found. Taken together, the lack of increased inflammatory cells, normal levels of the major lung antiprotease α1-AT, and normal levels of key MMPs, TIMPs, and Cathepsins in Tlr4^{-/-} mice indicated that TLR4 deficiency leads to increased lung elastolytic activity via alternative mechanisms.

### Experimental Example 3

### TLR4-Deficient Mice Show Decreased Antioxidant Activity

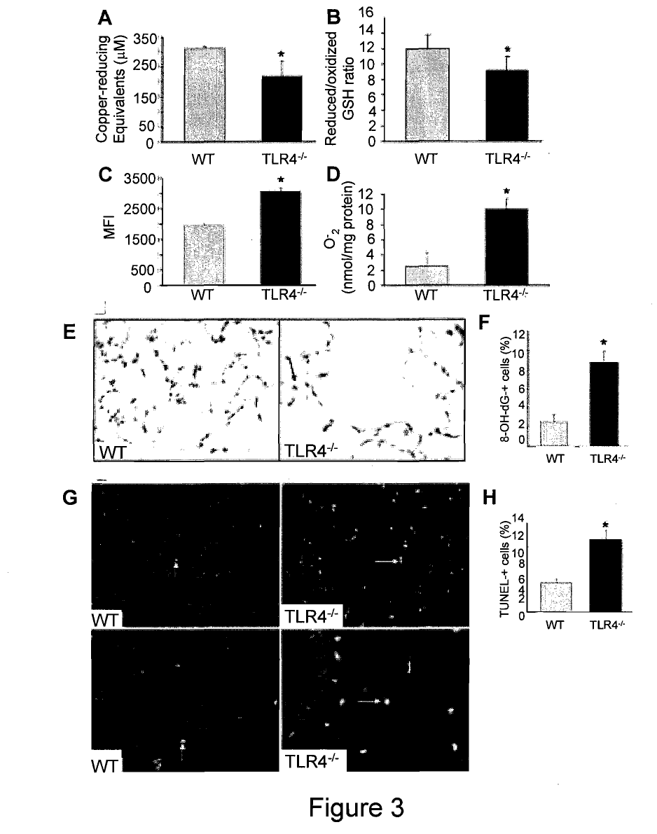

Increased ROS, or oxidant stress, not only is important in the pathogenesis of emphysema but also can directly inactivate antiproteases. Therefore, levels of oxidants in Tlr4^{-/-} mice were examined by measuring the total antioxidant capacity in BAL and serum. The total antioxidant capacity was dramatically decreased in both the BAL (FIG. 3A) and serum (data not shown) of Tlr4^{-/-} mice compared with WT mice. Consistent with the reduced antioxidant capacity, levels of reduced glutathione (GSH), the major antioxidant in the lung, were markedly decreased in BAL from Tlr4^{-/-} mice (FIG. 3B). It has been previously shown by the inventors of the instant invention that Nox-dependent formation of the oxidant superoxide (O₂⁻) plays an important role in oxidant-induced lung injury. Therefore, it was investigated whether TLR4 deficiency in vivo leads to altered Nox-mediated O₂⁻ formation. Using fluorescence microscopy and flow cytometry detection for dihydroethidine, a measurement of O₂⁻ anion, marked increases of O₂⁻ were found in isolated lung cells of Tlr4^{-/-} mice compared with WT mice (FIG. 3C). These Nox-mediated O₂⁻ measurements were confirmed with a cytochrome C reduction assay. Again, the production of O₂⁻ in isolated total lung cells from Tlr4^{-/-} mice was significantly higher than that of WT mice (FIG. 3D). These data suggested that Tlr4^{-/-} mice accumulated exaggerated levels of the ROS O₂⁻ by 3 months of age via Nox-mediated mechanisms.

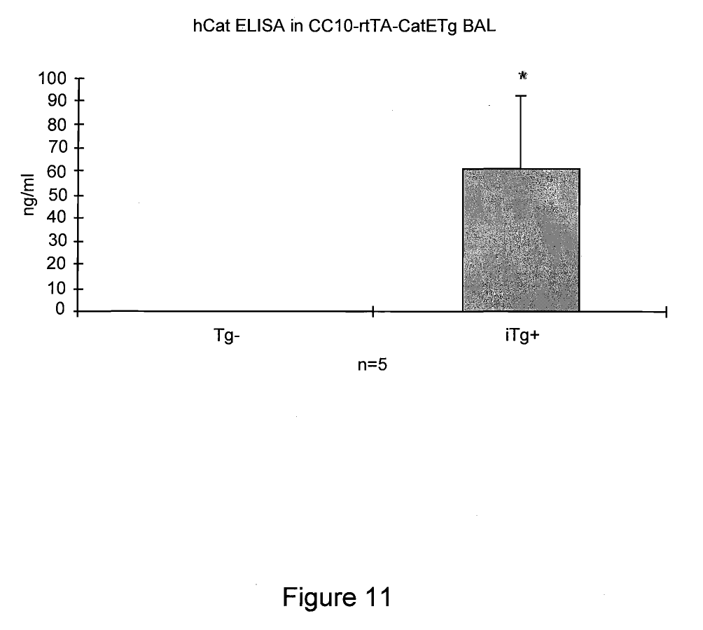

Targets of ROS include macromolecules such as lipids, proteins, and DNA, which become oxidized and lead to cellular dysfunction. Markers of lipid peroxidation have been found to be increased in patients with emphysema. Similarly, BAL from Tlr4^{-/-} mice showed increased lipid oxidation, as assessed by malondialdehyde and 4-hydroxyalkenals production (data not shown). Increased DNA oxidation in vivo was detected, as measured by levels of 8-hydroxy-2′-deoxyguanosine (8-OH-dG), the oxidized form of guanine (FIG. 3, E and F). Oxidants are key mediators of apoptosis, and lung apoptosis is thought to contribute to the development of emphysema in animal models and in humans. Therefore, Tlr4^{-/-} mice were evaluated for DNA strand breaks in situ by fluorescent TUNEL staining and found an increased number of TUNEL-positive cells in the lungs of Tlr4^{-/-} mice compared with WT lungs (FIG. 3, G and H). Results were confirmed using flow cytometric quantitation of TUNEL as well as annexin V-propidium iodide (annexin V-PI) staining in isolated lung cells of Tlr4^{-/-} mice, which showed significantly increased lung cell death (Supplemental FIG. 11). Next, the question of whether a caspase 3-mediated cell death was occurring in Tlr4^{-/-} mouse lungs was addressed. It was found that there were no differences in caspase 3 mRNA expression or levels of activated caspase 3 between WT and Tlr4^{-/-} mice. In addition, no differences were found in caspase 8 or caspase 9 activation. Thus far, data indicate that TLR4 deficiency leads to a pro-oxidant state and is associated with decreased antiprotease activity, increased elastin degradation, DNA oxidation, and cell death, and ultimately lung destruction.

### Experimental Example 4

### Antioxidants Reverse TLR4 Deficiency-Induced Emphysema

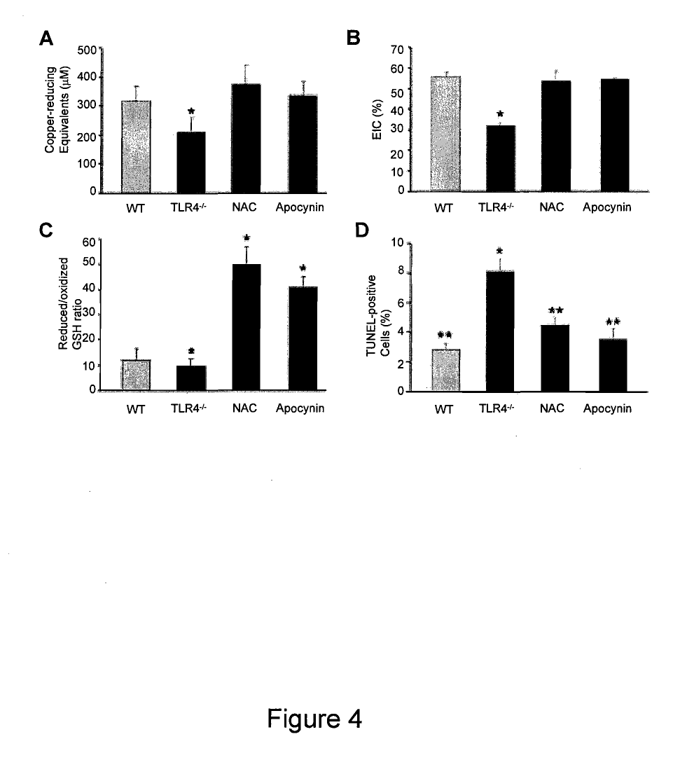

To determine whether increased oxidants are responsible for the emphysema phenotype in Tlr4^{-/-} mice, antioxidant treatment of the mice was attempted to rescue the mice. In addition to administering NAC, a GSH precursor, the Nox inhibitor apocynin was also administered because the present data suggested a Nox-mediated mechanism of ROS generation in TLR4 deficiency. Both NAC and apocynin treatment increased the total antioxidant content in BAL (FIG. 4A) and serum (data not shown) of Tlr4^{-/-} mice to the levels of WT mice. This was associated with an increase in the EIC and with increased GSH levels in Tlr4^{-/-} mice (FIG. 4). Antioxidant therapy also decreased the number of lung TUNEL-positive cells (FIG. 4D) and restored the antineutrophil elastase activity of α1-AT in Tlr4^{-/-} mice. Importantly, both NAC and apocynin treatment prevented TLR4-mediated emphysema (FIG. 5, AC). These data demonstrated a causal role for oxidants in the lung destruction observed in Tlr4^{-/-} mice.

### Experimental Example 5

### TLR4 Deficiency on Lung Parenchyma Induces Mouse Lung Emphysema

The contribution of cells from hematopoietic versus nonhematopoietic lineages in the pathogenesis of emphysema is unclear. Studies have shown that hematopoietic cell lineages, such as neutrophils, macrophages, and lymphocytes, play important roles in the pathogenesis of emphysema. However, studies have also shown that lung structural cells, such as endothelial and epithelial cells, are also involved in the pathogenesis of emphysema. The relative importance of TLR4 expression in these cell lineages was examined by creating bone marrow-chimeric mice. Tlr4^{-/-} mice were transplanted with WT bone marrow, which would restore TLR4 expression in hematopoietic cells but not in lung structural cells, and conversely, WT mice were transplanted with TLR4-deficient bone marrow, which would result in TLR4 expression in lung structural cells but not in hematopoietic cells. WT bone marrow was observed to not rescue Tlr4^{-/-} mice from emphysema and Tlr4^{-/-} bone marrow did not induce emphysema in WT mice (FIG. 5D). This indicates that TLR4 deficiency in nonhematopoietic cells (such as endothelial, epithelial, and fibroblast cells, which have been implicated in the pathogenesis of emphysema) is responsible for emphysema. Lung endothelial cells for subsequent studies were focused on because (a) endothelial cells account for the majority (46%) of all lung cells; (b) endothelial cells are important in the pathogenesis of emphysema as well as in oxidant responses; (c) endothelial cells have elastase-like activities; and (d) endothelial cells express TLR4 (Faure E., et al., 2000, J. Biol. Chem. 275:11058-11063).

### Experimental Example 6

### Nox Regulates the Increased Elastolytic Activity in Tlr4^{-/-} Lung Endothelial Cells

Mouse lung endothelial cells (MLECs) were isolated from Tlr4^{-/-} mice exhibit increased elastolytic activity. Tlr4^{-/-} MLECs showed markedly increased elastolytic activity compared with WT MLECs (FIG. 6A). In order to confirm that the absence of TLR4 in endothelial cells leads to increased elastolytic activity, siRNA was used to knock down endogenous TLR4 expression (FIG. 6B). MLECs transfected with TLR4 siRNA exhibited significantly increased elastolytic activity compared with WT MLECs and MLECs transfected with nonspecific siRNA (FIG. 6C).

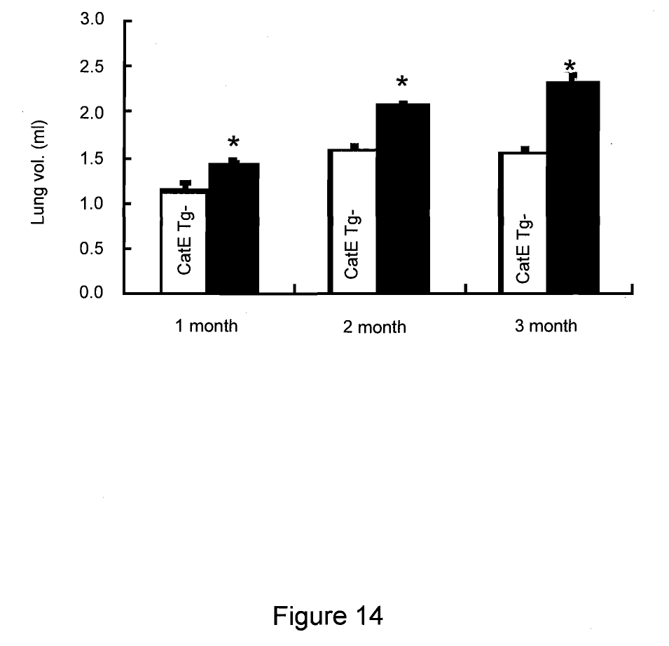

Although apocynin is widely used as a Nox inhibitor in vitro and in vivo, our data was confirmed with a second Nox inhibitor, diphenylene iodonium (DPI). Consistent with the inventors' in vivo data using NAC and apocynin (FIG. 4), the elastolytic activity observed in Tlr4^{-/-} MLECs was reversed to levels achieved by the broad-acting antioxidant NAC using apocynin or DPI (FIG. 6A). These data suggested that a Nox-mediated mechanism was responsible for the increased oxidants observed in Tlr4^{-/-} MLECs. Nox is a multimeric enzyme system of which Nox2 (gp91^{phox}) is the most commonly described catalytic component, especially in phagocytic cells. Recently, several novel homologs of Nox2 have been described in a variety of nonphagocytic cells (Nox1-Nox5, Duox1, and Duox2). Nox1 and Nox3 appear to be the closest structural homologs of Nox2, but the precise function and tissue distribution of these new Nox members are poorly defined. To determine whether TLR4 deficiency regulates Nox, mRNA expression of various Nox proteins was examined in lung lysates from Tlr4^{-/-} and WT mice. Nox3 was present at a very low level in WT lungs, whereas Tlr4^{-/-} lung lysates showed increased expression of Nox3 mRNA and protein (FIG. 7, A and B). Nox1, -2, and -4 expression levels were similar between Tlr4^{-/-} and WT lungs (Supplemental FIG. 14). Next, Nox3 mRNA expression was examined in MLECs and found that, similar to the lung lysates, Tlr4^{-/-} MLECs expressed increased Nox3 mRNA expression (FIG. 7C). In order to confirm that the increased Nox3 expression is due to the absence of TLR4 in MLECs, TLR4 siRNA was used to knock down TLR4 expression in WT MLECs (FIG. 6B). MLECs transfected with TLR4 siRNA expressed increased Nox3 mRNA compared with untreated control and nonspecific siRNA treated MLECs (FIG. 7D). TLR4 deficiency-mediated Nox3 expression was examined to determine if it was responsible for increased elastolytic activity. Nox3 siRNA was used to knock down Nox3 expression in Tlr4^{-/-} MLECs (FIG. 7E) and then examined levels of elastolytic activity. Nox3 siRNA significantly decreased the elastolytic activity observed in Tlr4^{-/-} MLECs compared with untransfected or nonspecific siRNA transfected MLECs (FIG. 7F). As expected, MLECs transfected with TLR4 siRNA exhibited increased elastolytic activity to levels comparable to that of MLECs isolated from Tlr4^{-/-} mice (FIG. 7F). These data indicated that TLR4 deficiency leads to increased Nox activity via the induction of Nox3 and, consequently, increased elastolytic activity in MLECs.

### Experimental Example 7

### Lung-Targeted Transgenic Overexpression of Cathepsin E Results in Emphysema In Vivo

Cathepsin E is regulated by TLR4. In Tlr4^{-/-} mice there is increased Cathepsin E expression in vitro and in vivo. Microarray gene expression analysis (Affymetrix Inc.) on lung lysates isolated from 3 month old Tlr4^{-/-} and wild type mice revealed a 3-fold induction in Cat E mRNA expression in TLR4−/− lung lysates, confirmed with quantitative real-time PCR (qRT-PCR) analysis of lung lysates isolated from mice of different ages. Cat E induction preceded the development of overt emphysema by months. Cat E protein expression was also increased in 3 month old TLR4−/− lung lysates. Cat E protein was detected using ELISA in BAL and serum of TLR4−/− mice, indicating that it is secreted. Immunohistochemical staining for Cat E demonstrated that various cell types express Cat E protein in TLR4−/− mice.

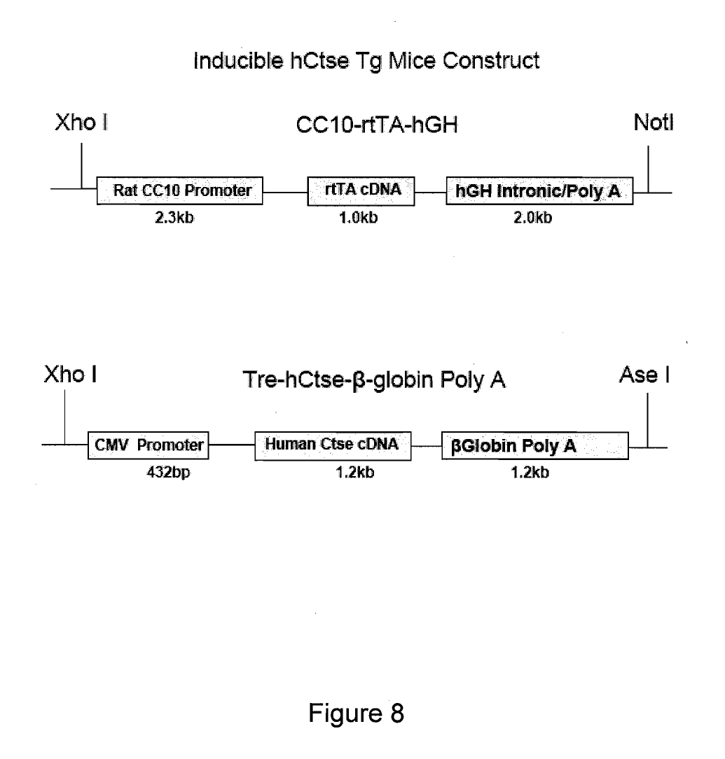

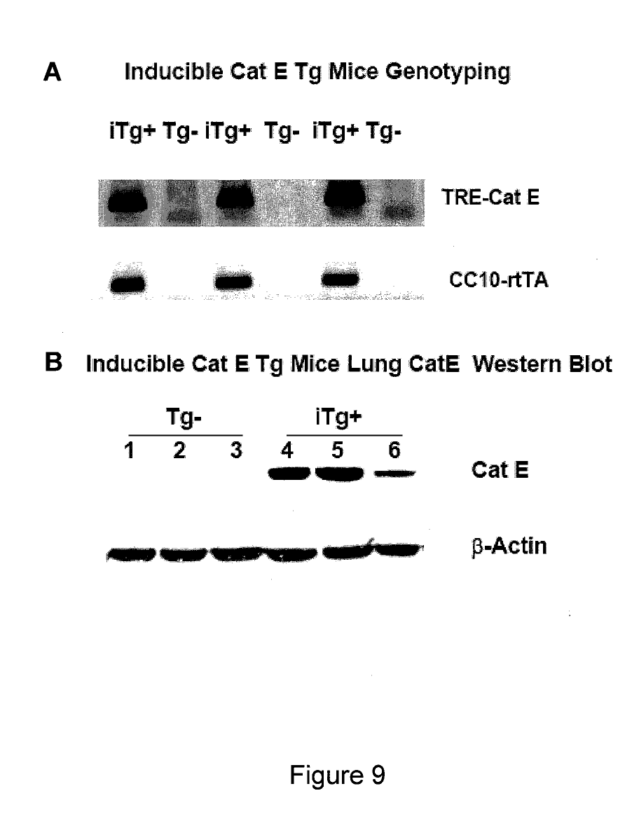

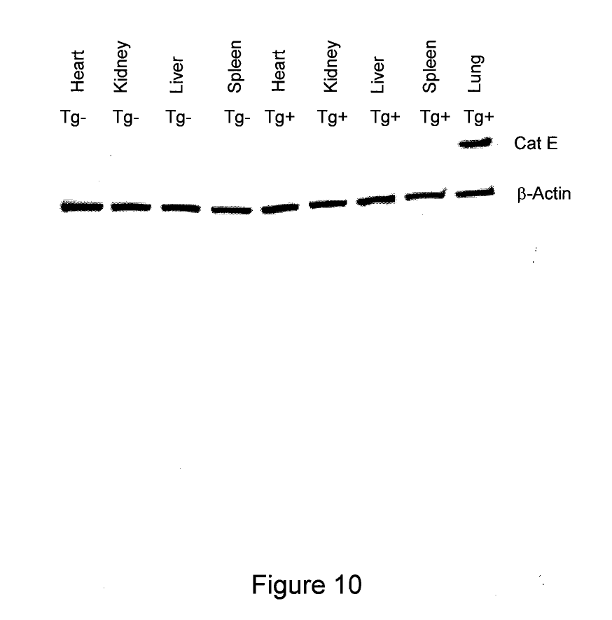

Transgenic mice with lung-targeted overproduction of Cathepsin E (Cathepsin E Tg⁺) were constructed to generate inducible, human Cathepsin E transgenic mice (iTg+), which when fed doxycycline-containing water will induce the human Cathepsin E transgene and result in increased levels of Cathepsin E protein (FIG. 8). These trangenic animals were genotyped (FIG. 9A) and Western blot analysis was used to confirm that inducible and increased levels of human Cathepsin E protein could be detected in iTg+ mice compared to Tg− mice (FIG. 9B). β-Actin levels were used to control for protein loading. Inducible Cathepsin E production was specific to the lung in Cathepsin E iTg⁺ mice (FIG. 10).

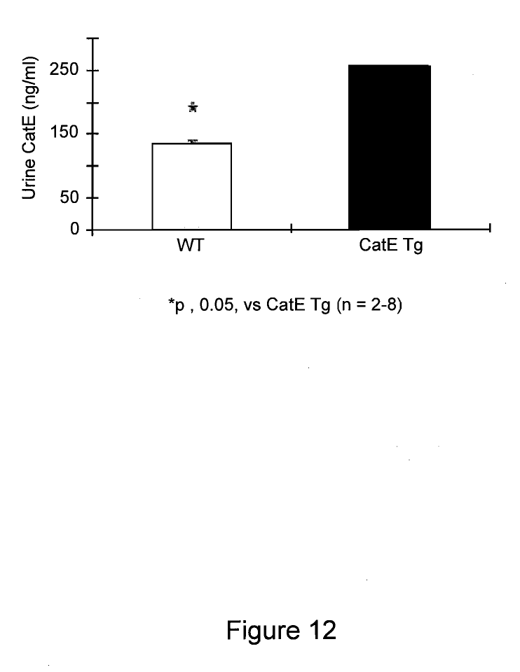

Cathepsin E is a secretable protein and was detectible in various, accessible biological compartments of Cathepsin E iTg+ mice. Broncho-alveolar lavage fluid (BAL) obtained from Cathepsin E iTg⁺ mice was tested for Cathepsin E protein (FIG. 11). Cathepsin E levels were significantly increased in BAL from Cathepsin E iTg⁺ mice as compared to Tg− mice. In addition, transgenic mice that expressed constitutively active Cathepsin E protein in a lung-targeted manner (Cathepsin E Tg+) show increased levels of Cathepsin E detectable in urine compared to wildtype (WT) mice FIG. 12). This indicates that Cathepsin E is a secreted molecule and can potentially be a useful marker that can be detected in urine samples from people.

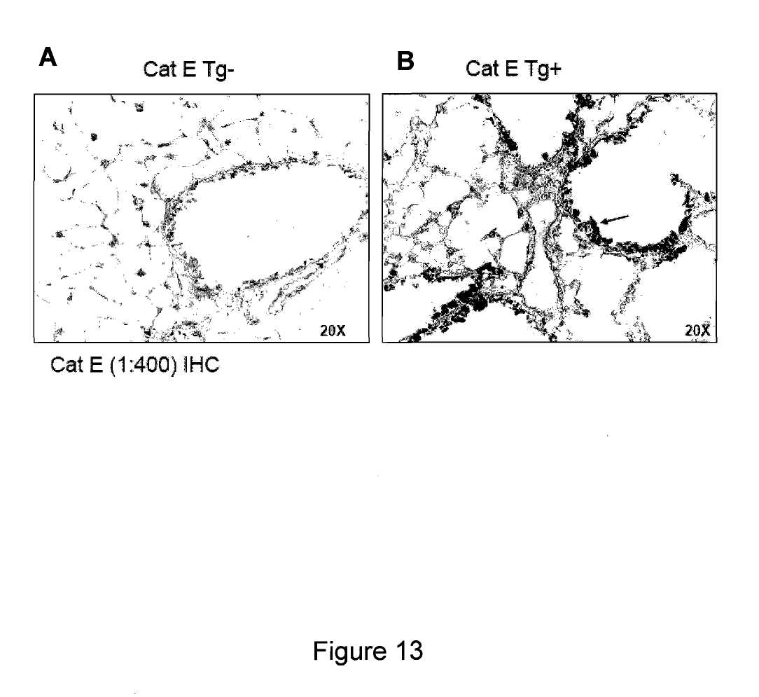

The lungs of Cathepsin E iTG+ mice exhibit numerous pathological changes in lung tissue histology and morphometry that are consistent with emphysema. Immunohistochemical staining of lung sections from wild type (Cathepsin E Tg−; FIG. 13A) and Cathepsin E Tg+ mice show increased levels of Cathepsin E protein in the lung epithelial cells of Cathepsin E Tg+ (FIG. 13B) as compared to Cathepsin E Tg− mice.

Histologic evaluation of lung tissue obtained from transgenic mice with lung-targeted Cathepsin E overexpression revealed characteristic enlargement of the air spaces distal to the terminal bronchioles accompanied by destruction of the normal alveolar architecture typical of emphysema. Littermate controls (Cathepsin E Tg⁻) exhibited normal lung morphology. Quantification revealed significantly increased lung volume in Cathepsin E Tg⁺ mice as compared to Cathepsin E Tg⁻ controls over a three month period (FIG. 14), indicating that Cathepsin E has the biologic ability to cause lung-destructive changes, as seen in emphysema.

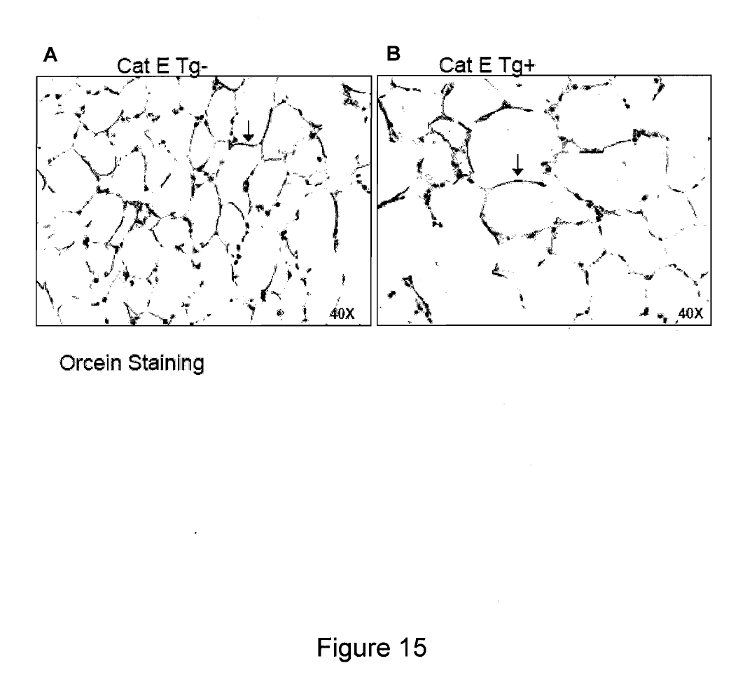

Histochemical analyses for elastin in the elastic fibers as assessed by orcein and elastin staining showed thin, fragmented elastin segments in Cathepsin E Tg⁺ mice, whereas the elastic fibers in Cathepsin E Tg⁻ mice appeared intact (FIG. 15).

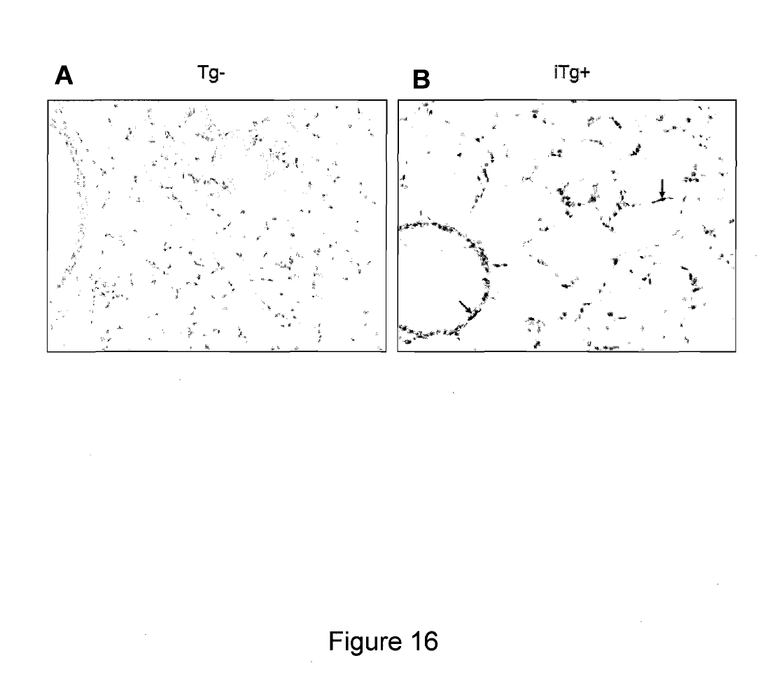

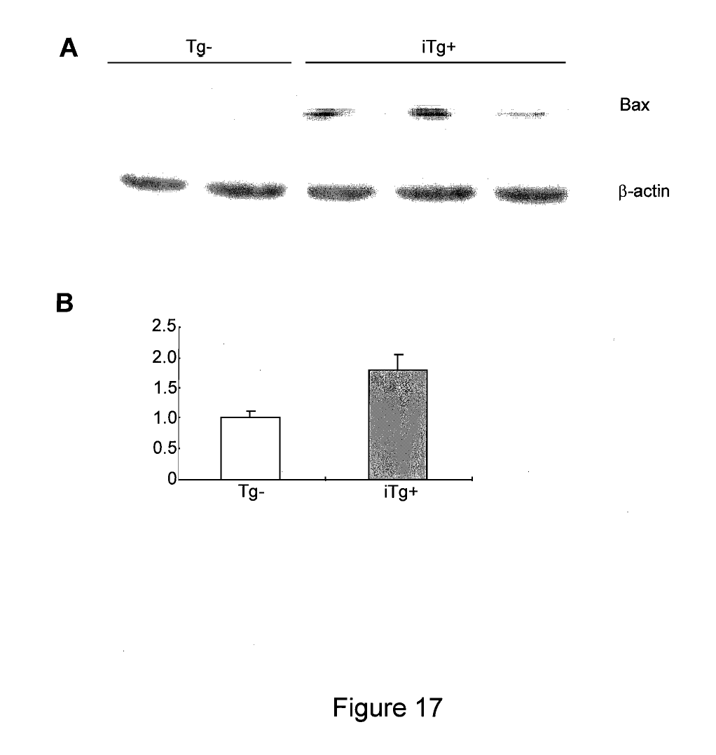

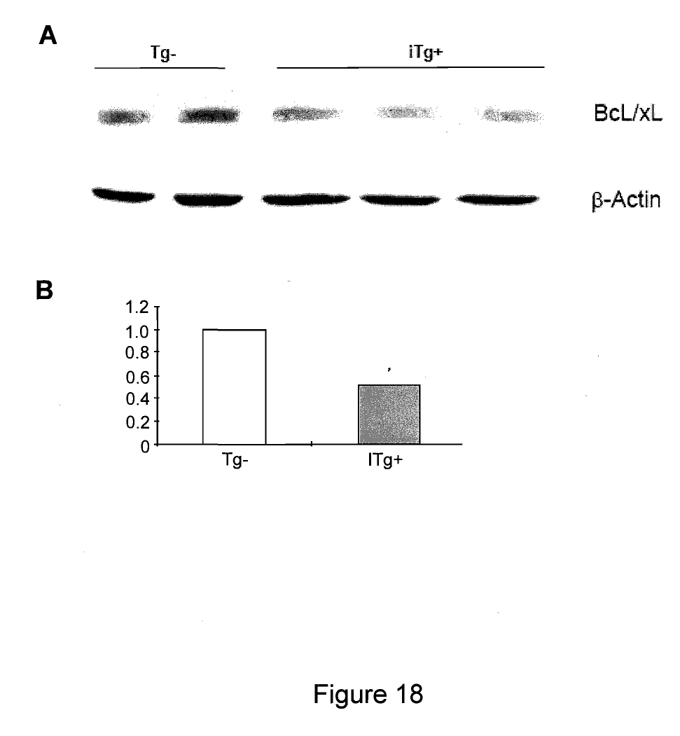

In order to determine potential mechanisms of Cathepsin E-induced lung destruction, TUNEL staining on lung sections was performed to detect levels of apoptosis in the lung (FIG. 16). Increased lung apoptosis has been associated with the development of emphysema. It is demonstrated herein for the first time that Cathepsin E may have apoptotic effects in the lung. Mice that are iTg+, in which Cathepsin E expression is induced, show increased numbers of TUNEL-positive/apoptotic cells (arrows) compared to Tg− mice. In addition, increased levels of the pro-apoptotic protein Bax is detected in the lungs of Cathepsin E iTg+ mice as compared to Tg− mice (FIG. 17), while decreased levels of the anti-apoptotic protein Bcl/xL is detected in the lungs of Cathepsin E iTg+ mice as compared to Tg− mice (FIG. 18). These data confirm that lung apoptosis and cell death may be regulated by Cathepsin E. Thus it is demonstrated herein for the first time that Cathepsin E may induce lung emphysema by activating lung cell apoptosis and cell death.

### Experimental Example 8

### siRNA Silencing of Cathepsin E

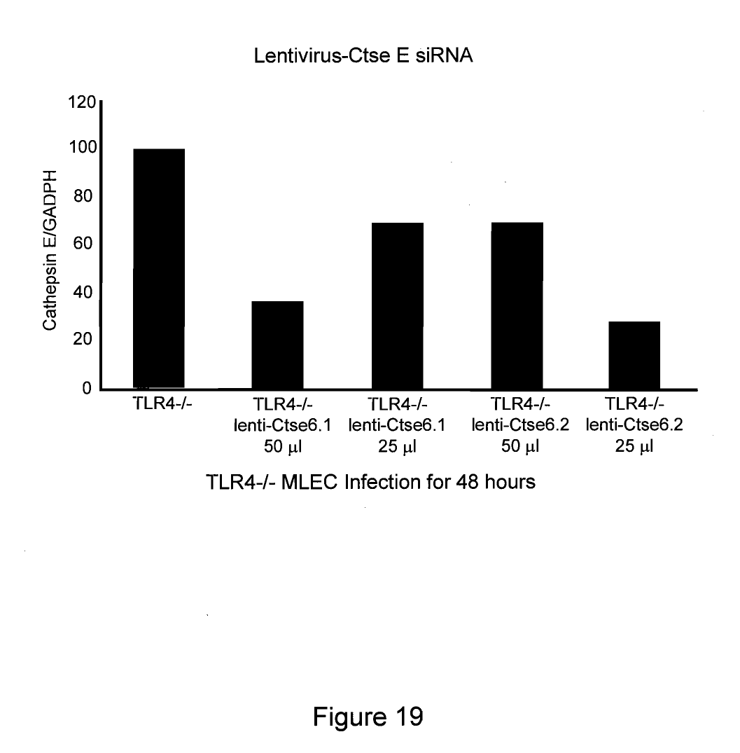

Cathepsin E mRNA can be silenced in lung endothelial cells using lentiviral Cathepsin E siRNA as shown in FIG. 19.

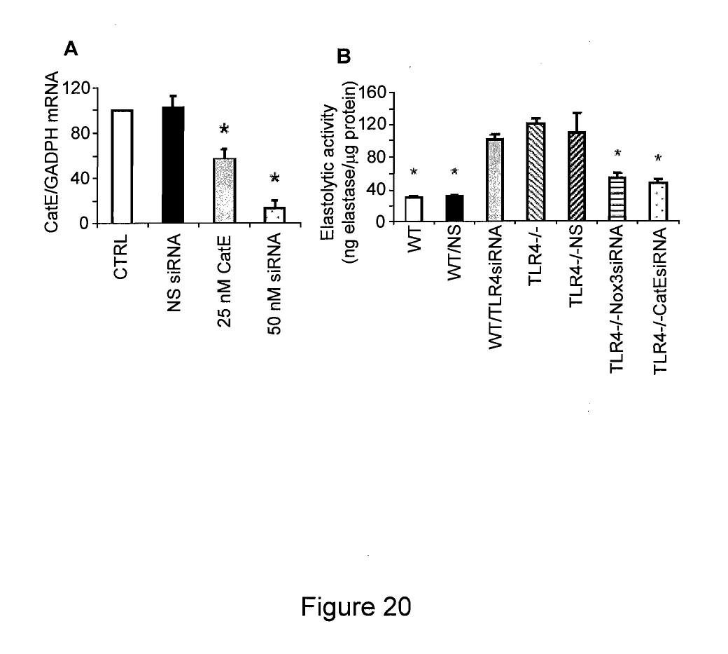

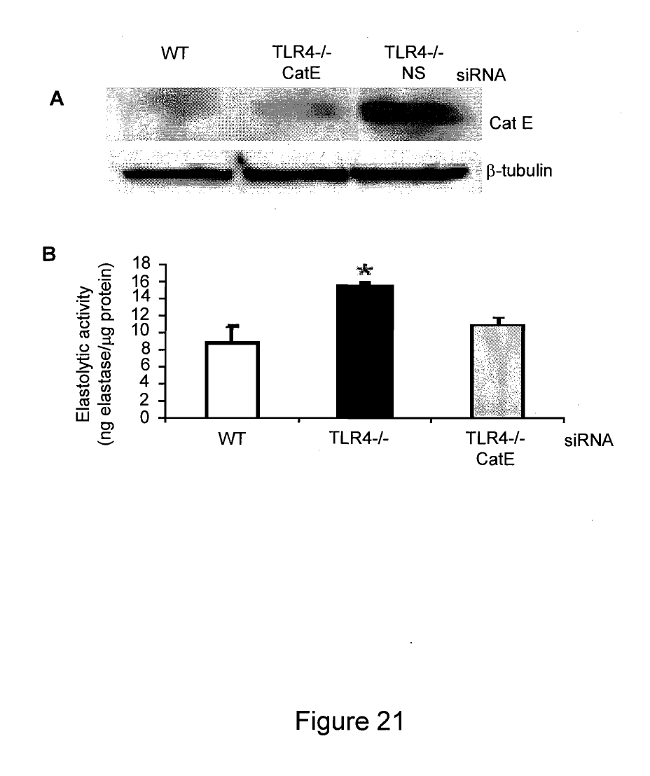

Inhibiting Cathepsin E expression in TLR4−/− Ec significantly decreased elastolytic activity in TLR4−/− Ec, indicating that both Nox3 and Cathepsin E are responsible for the increased elastolytic activity in TLR4−/− Ec. Cat E siRNA were designed using methods previously described and incorporated herein in its entirety (Zhang et al., 2004, J. Biol. Chem. 279:10677-10684). A dose-response was performed by transfecting Ec with Cathepsin E siRNA (FIG. 20A). At the optimal dose (50 nM), Cathepsin E siRNA had no effect on Nox3 mRNA expression, which confirmed the specificity of the siRNA but also suggested that Cat E expression is not upstream of Nox3 induction. Consistent with previous results, wild type Ec transfected with TLR4 siRNA exhibited increased elastolytic activity (comparable to TLR4−/− Ec) while Nox3 siRNA significantly decreased elastolytic activity in TLR4−/− EC (FIG. 20B). Intranasal siRNA administration is also effective in vivo, as evidenced by significant Cathepsin E protein inhibition in TLR4−/− lung lysates compared to non-specific (NS) siRNA administration (FIG. 21A). Consistent with in vitro elastolytic data presented herein, Cathepsin E siRNA in vivo reduced the elastolytic activity detected in the BAL (FIG. 21B) and lung lysates of TLR4−/− mice.

### Experimental Example 9

### Mechanisms of Cathepsin E-Induced Lung Destruction

Cathepsin E is an aspartic protease that requires an acidic environment for optimal activity. Therefore, Cathepsin E activity was assessed in lung lysates isolated from WT and Cathepsin E Tg+ mice using acid (pH=3.5) and neutral (pH=7.4) assay conditions.

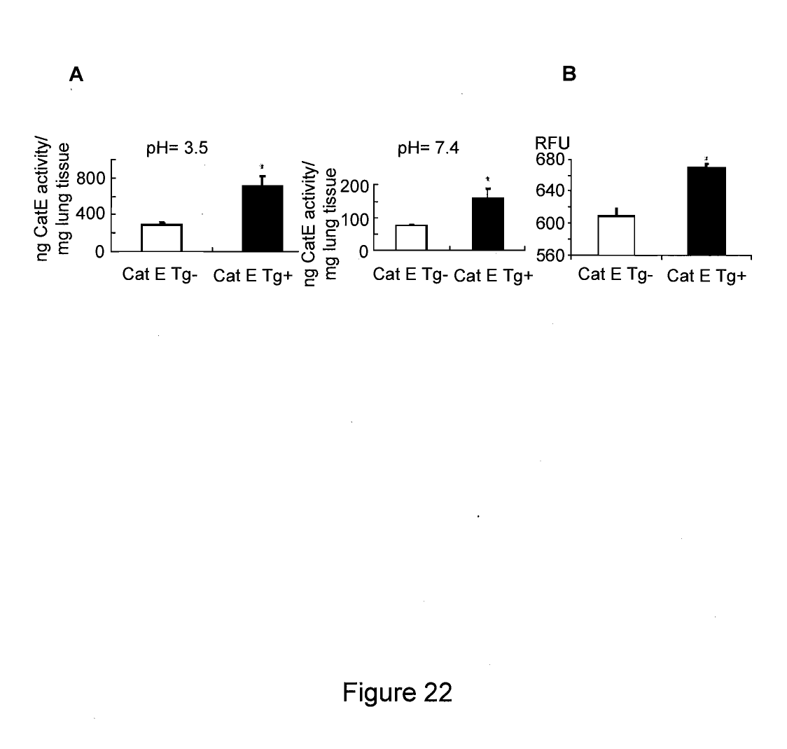

A significant increase in Cathepsin E activity was detected in Cat E Tg+ mice regardless of pH, but overall Cat E activity was higher at an acid pH (FIG. 22A). Of note, we assessed BAL pH, using unbuffered saline, in WT, TLR4−/−, and Cat E Tg+ mice and detected BAL pH=6.0-6.4 in all mice tested, which may partially account for acid pH-sensitive proteases, like Cat E, to be active. Since elastin is not a direct substrate for Cat E proteolytic cleavage, Cat E may have indirect protease activity. We did not detect increased MMP2 or 9 activity in Cat E Tg+ mice (data not shown). Interestingly, we detected increased carboxypeptidase A activity in lung lysates isolated from Cat E Tg+ mice (FIG. 22B).

### Experimental Example 10

### Cathepsin E Expression is Associated with Cigarette Smoking in Humans

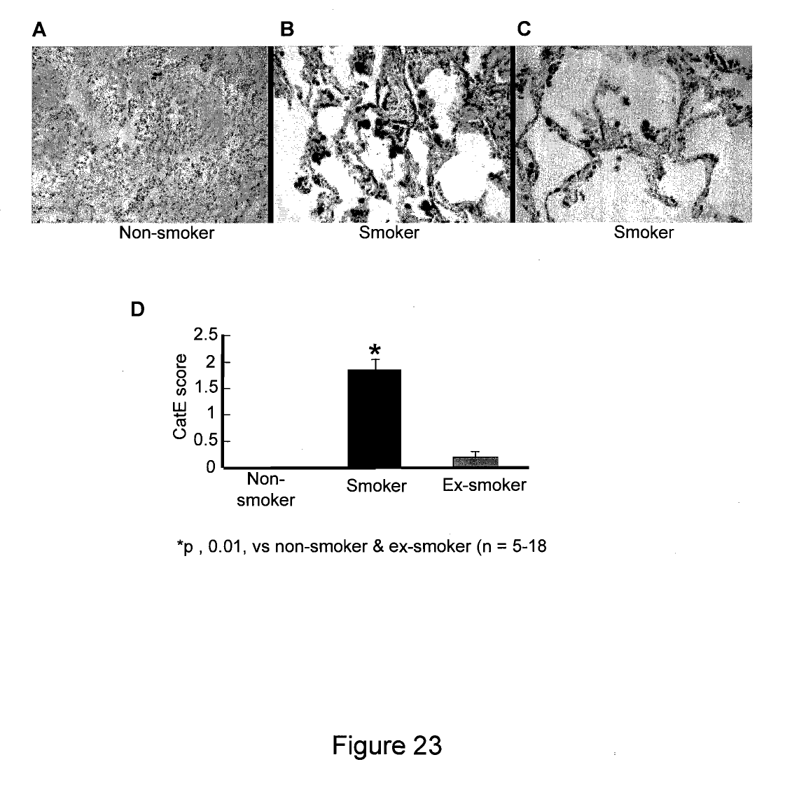

In lung sections obtained from people, Cathepsin E expression levels were strongly associated with cigarette smoking. FIG. 23 compares lung tissue sections obtained from a non-smoker (FIG. 23A) and two smokers (FIG. 23B and FIG. 23C). Cathepsin E staining is elevated in the two smokers as are the histologic destruction of lung tissue. FIG. 23 D provides a quantification of Cathepsin E expression (Cathepsin E score) in lung sections obtained from a non-smoker, a smoker, and an ex-smoker.

### Experimental Example 11

### Cathepsin E Expression is Associated with Emphysema/COPD

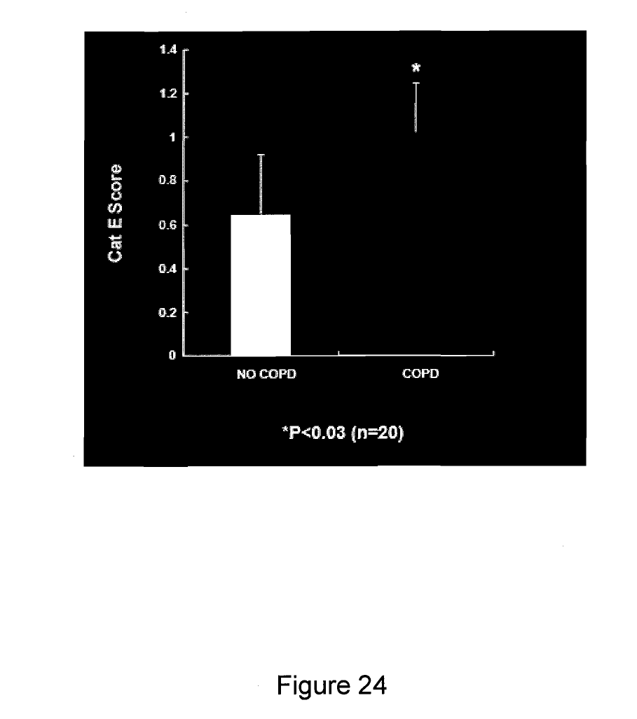

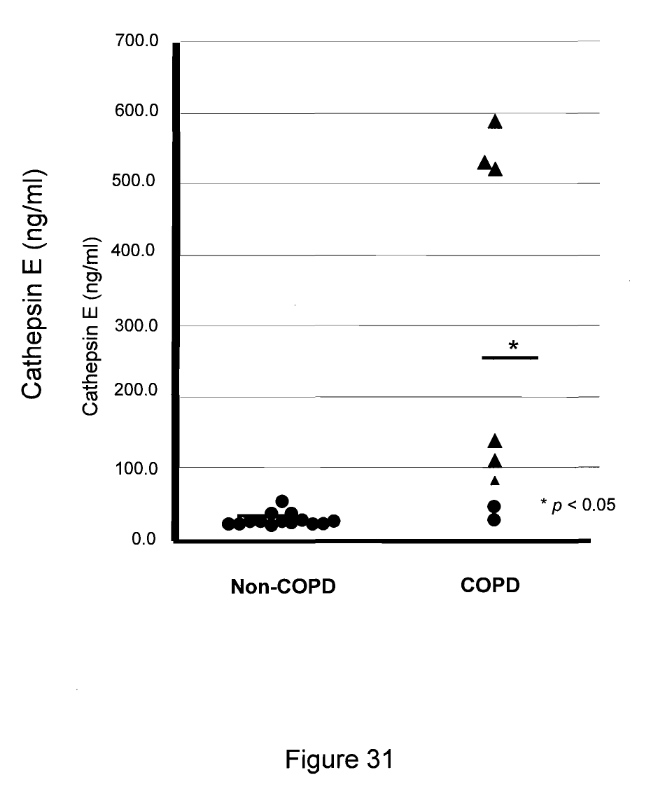

In lung sections obtained from people, Cathepsin E expression levels were associated with COPD, of which emphysema is a major subset. FIG. 24 compares Cathepsin E expression (Cathepsin E score) in lung sections obtained from people without COPD and people diagnosed with COPD. The patients with COPD had a significantly higher Cathepsin E score than those without COPD (N=20, p<0.03). FIG. 31 shows significantly elevated serum cathepsin E levels in patients with COPD as opposed to patients without COPD COPD (N=20, p<0.03).

### Experimental Example 12

### TLR4Deficiency Leads to Increased Nox3 in EC and Lung

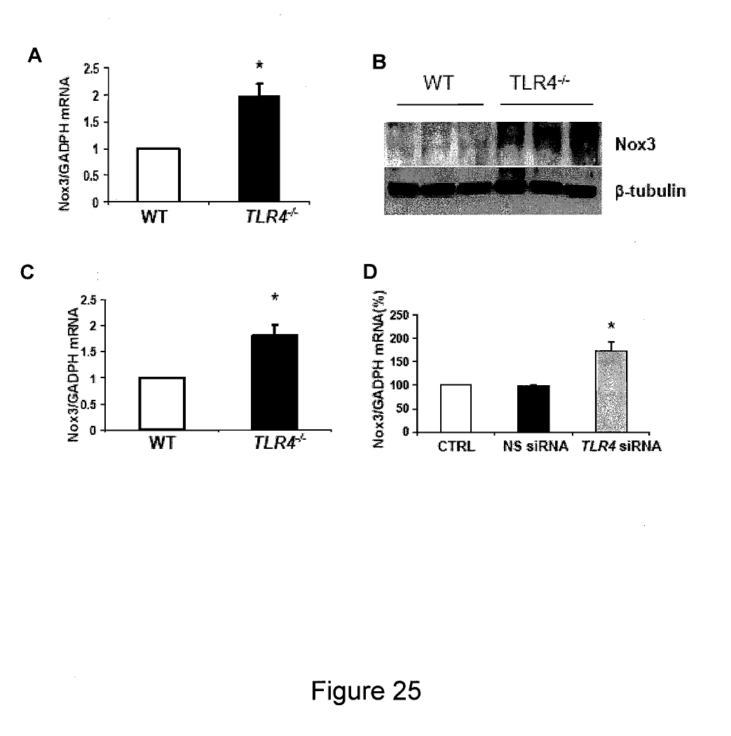

mRNA expression of Noxs 1-4, the most widely distributed Nox proteins was examined. Nox3 was present at a very low level in WT lungs, whereas TLR4−/− lung lysates showed increased expression of Nox3 mRNA and protein (FIG. 25 A and FIG. 25B). Nox 1, 2, and 4 expression levels were similar between TLR4−/− and WT lungs. Consistent with the lung lysate data presented herein, EC isolated from TLR4−/− lungs expressed increased Nox3 mRNA expression (FIG. 25C). In order to confirm that the increased Nox3 expression was due to the absence of TLR4 in EC, TLR4 siRNA was used, which increased Nox3 mRNA expression compared to untreated control and NS siRNA-treated Ec (FIG. 25D).

### Experimental Example 13

### Increased Nox3 Expression Leads to Increased Elastolytic Activity in EC

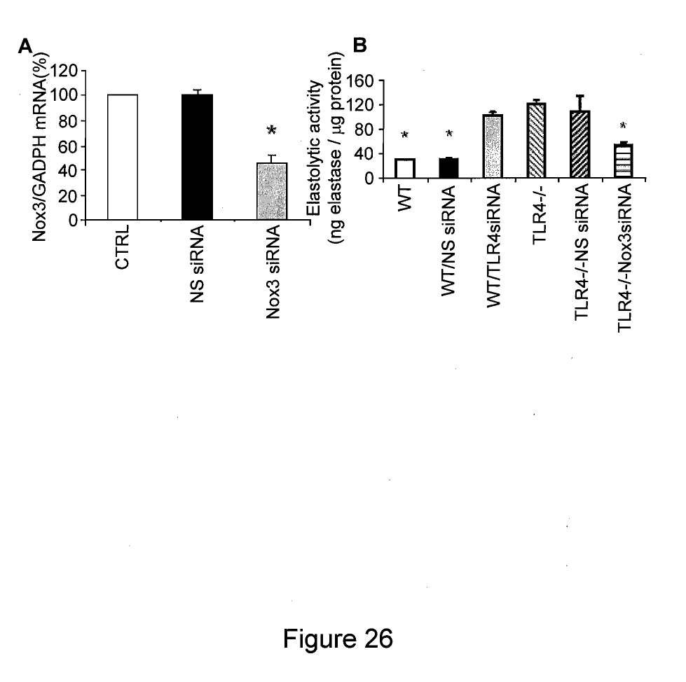

It was then examined whether TLR4 deficiency mediated Nox3 expression was responsible for increased elastolytic activity in EC. After confirming Nox3 inhibition by Nox3 siRNA (FIG. 26A), Nox3 siRNA was found to significantly decreased the elastolytic activity observed in TLR4−/− Ec compared to naive or NS siRNA treated Ec (FIG. 26B). As expected, WT EC transfected with TLR4 siRNA exhibited increased elastolytic activity to levels comparable to that of TLR4−/−. These data indicate that TLR4 deficiency leads to increased Nox activation via the induction of Nox3 expression and, consequently, increased elastolytic activity in Ec. Of note, increased Nox3 expression was detected in TLR4^{-/-} mice at 2 months of age, prior to the development of emphysema, while Nox1, 2 and 4 expression remained unchanged compared to WT mice.

### Experimental Example 14

### Cellular Localization of Nox3 Expression in TLR4 Deficient Lungs

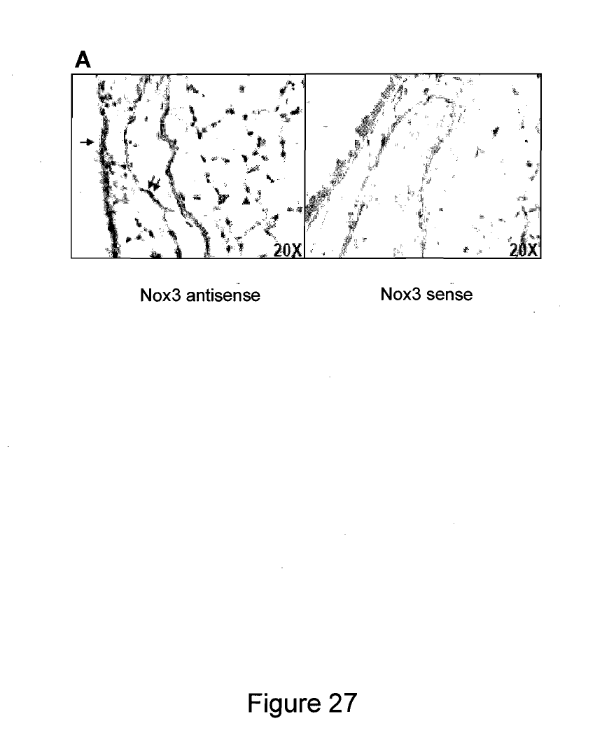

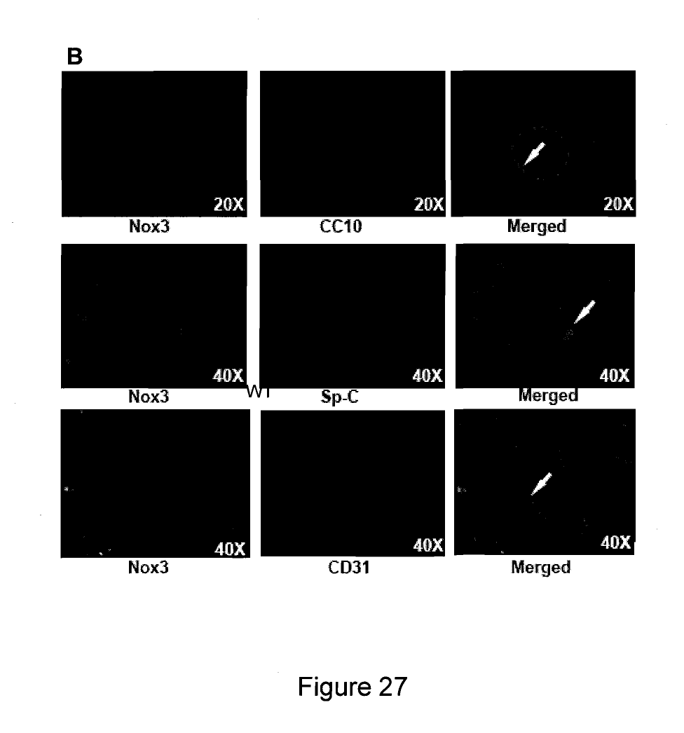

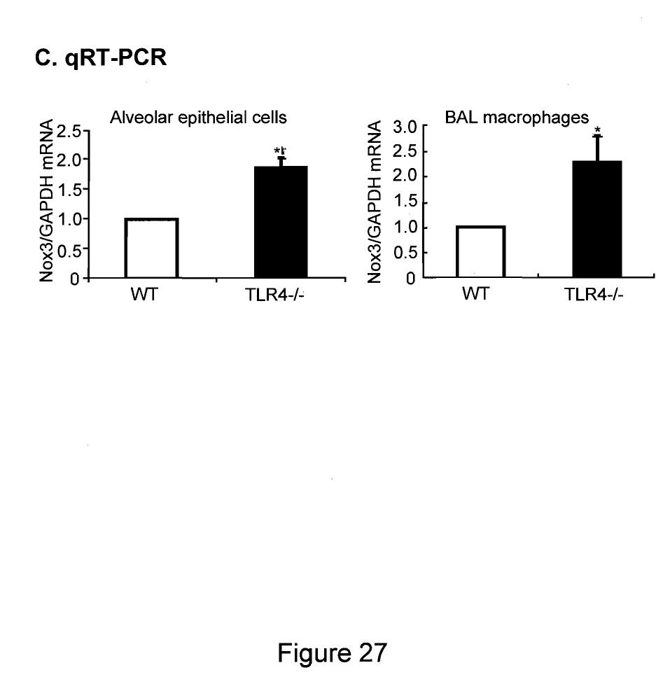

In order to identify the cell type(s) that express Nox3 in TLR4−/− mouse lungs, in situ hybridization was performed using anti-Nox3 probes, which detect Nox3 mRNA expression in vivo. The dark blue staining of airway, alveolar, and endothelial cells with anti-sense Nox3 indicates the presence of Nox3 mRNA expression (FIG. 27A). A sense Nox3 probe was used as a negative control. In situ results were confirmed using antibodies directed against Nox3 and specific cell types. Nox3 staining was specifically visualized in airway epithelium, type II alveolar epithelium, and endothelium in the merged images, indicating the presence of Nox3 in these cell types (FIG. 27B). Furthermore, primary type II alveolar epithelial cells and BAL macrophages were isolated from TLR4−/− mouse lungs and confirmed elevated Nox3 mRNA expression compared to WT cells (FIG. 27C). It has already been shown that lung Ec isolated from TLR4−/− mice express increased Nox3 mRNA in FIG. 25. Therefore, these data indicate that multiple cell types, including lung structural cells, induce Nox3 in the setting of TLR4 deficiency.

### Experimental Example 15

### Distribution of Intranasal Lentiviral Administration

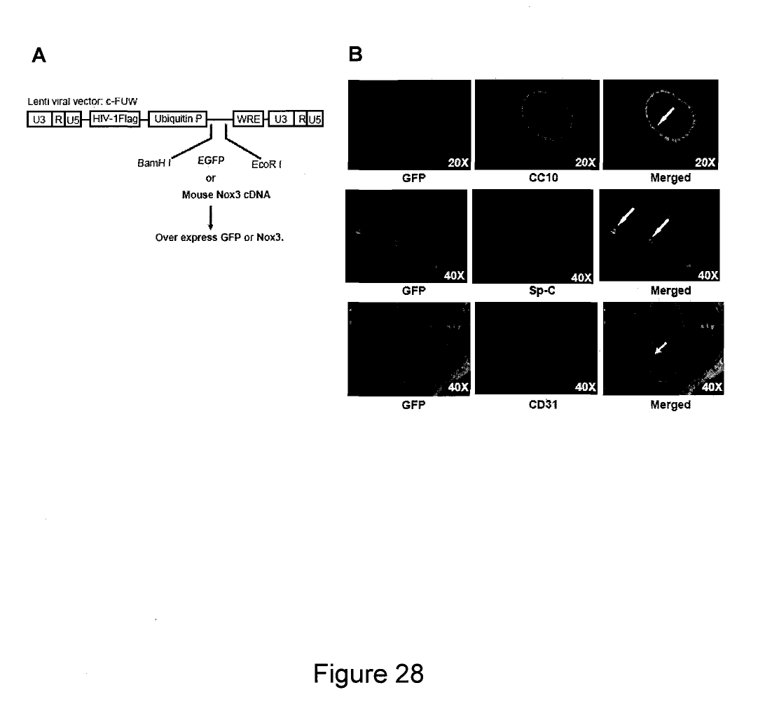

Intranasal viral vectors are an effective, lung-targeted method of modulating gene expression and can provide initial proof-of-concept studies while genetic mouse models are being created. It is demonstrated herein that intranasal viral administration can reach lung endothelium. Recently, intratracheal lentiviral vectors were observed to be effective over 6 weeks in the lung, which is consistent with the ability of lentiviruses to incorporate into the host genome and is useful for experiments re-quiring gene modulation for weeks to months (Hendrickson et al., 2007) Am. J. Resp. Cell Mol. Biol. 37:414-423). A GFP-tagged lentiviral vector was constructed (FIG. 28A) and the efficiency of transfection in vitro was tested. In order to visualize its distribution in airway, type II alveolar, and lung endothelial cells, intranasal lentiviral GFP vector (lenti-GFP) was delivered to WT mice and immunofluorescence analyses was performed to detect GFP (FIG. 28B). The GFP panels (left) show the detection of lenti-GFP, the cell-specific panels (middle) identify specific cell types and the merged images (right) indicate the presence lenti-GFP in specific cell types (FIG. 28B).

These results confirm that lentiviral vectors can be detected in a variety of lung compartments and is consistent with the robust gene induction reported with viral vectors in the lung (Zhang et al., 2006, FASEB J. 20:2156-2158; Siner et al., 2007, FASEB J. 21:1422-1432).

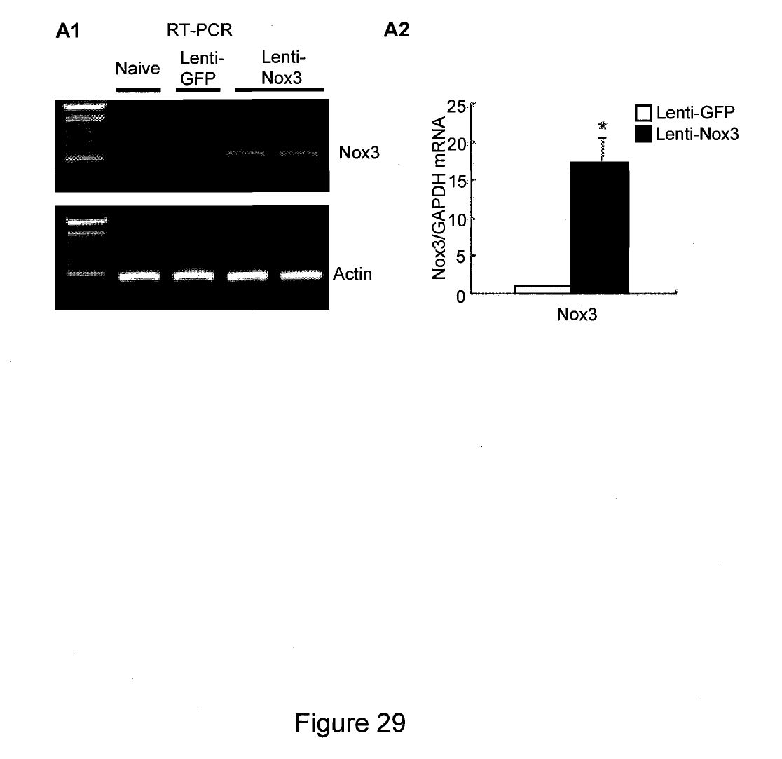

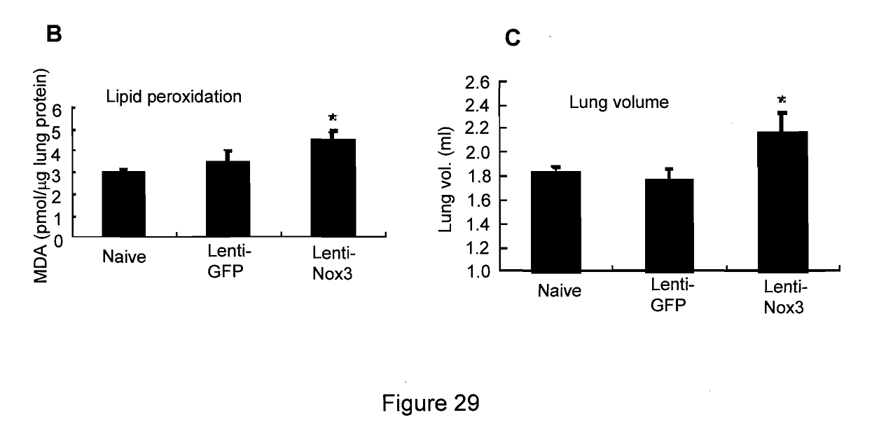

A lentiviral Nox3 (lenti-Nox3) was constructed in order to achieve lung-targeted Nox3 overexpression. Mice administered intranasal lenti-Nox3 at 1 month of age expressed increased Nox3 mRNA in WT (FIG. 29A). Lenti-GFP was used as a negative control. Lenti-Nox3 increased lung lipid peroxidation (FIG. 29B), a reflection of oxidant generation, as well as lung volumes in WT mice by 3 months of age (FIG. 29C).

### Experimental Example 16

### Cathepsin E and Nox3 are Regulated by Distinct Pathways Downstream of TLR4

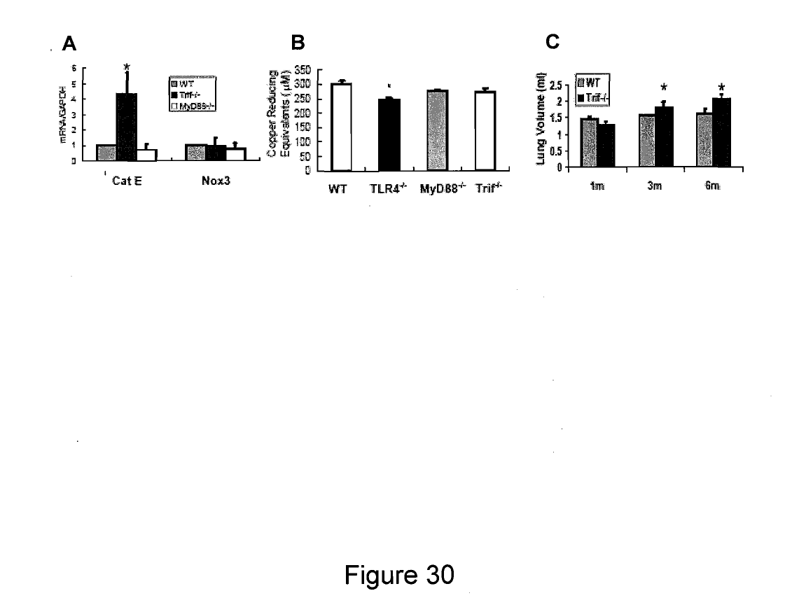

In order to determine the relevant downstream TLR4 pathway for Cathepsin E induction, qRT-PCR was initially performed on lung lysates isolated from MyD88−/− mice (C57BL6), which have previously been shown to develop emphysema (Zhang et al., 2006, J. Clin. Invest. 116:3050-3059). MyD88−/− lung lysates did not exhibit increased Cathepsin E nor Nox3 expression (FIG. 30A). A MyD88-independent adapter, Trif, may be involved. Trif−/− lungs (C57BL6). Trif exhibited significant Cathepsin E mRNA induction, comparable to that of TLR4−/− mice. However, Trif−/− lungs did not express increased Nox3 expression nor oxidant generation (FIG. 30A and FIG. 30B), indicating that Cat E and Nox3 expression are controlled by distinct pathways downstream of TLR4.

Consistent with the premise that a Trif-dependent pathway to emphysema is involved in Cat E regulation, Trif−/− mice exhibited increased lung volumes by 3 months (FIG. 30C). Similar to TLR4−/− mice, Trif−/− lungs do not exhibit significant lung inflammation. Collectively, these findings indicate that both MyD88-dependent and Trif-dependent pathways to emphysema exist but that Cathepsin E-mediated emphysema is Trif-dependent.

Thus, it is demonstrated herein that 1) TLR4 is required to suppress Cathepsin E expression in the lung, BAL, and serum; 2) lung structural cells (Ec and epithelium) express Cathepsin E; 3) Cathepsin E may have protease activity via other proteases, such as carboxypeptidase A, which may be responsible for TLR4- and Trif-deficiency-induced emphysema; 4) Cathepsin E Tg mice develop emphysema; and 5) Cathepsin E and Nox3 represent distinct TLR4-regulated pathways important in maintaining the structural integrity of the lung and which, when dysregulated increase an individual's susceptibility to emphysema/COPD.

### Experimental Example 17

### Role of Other TLRs in Emphysema/COPD

Other Toll-like receptors also play important roles in maintaining lung structural integrity. TLR1 deficiency leads to emphysema/COPD, much like TLR4 deficiency.

TLR2 deficiency counters the emphysema pathogenesis observed in TLR4 deficiency. Thus, TLR2 agonists or activators may represent valuable clinical targets in the treatment of emphysema/COPD.

The effects are specific, however: TLR3 and TLR9 deficiency do not lead to emphysema pathogenesis.

### Experimental Example 18

### Role of Nox3 in Emphysema and COPD

A transgenic, inducible mouse that leads to Nox 3 overexpression was developed. Induction of Nox 3 overexpression or TLR4−/− knockouts in lung of transgenic mice both lead to increased lung volume (emphysema) as compared to wild type mice (p<0.05). However, Nox 3 deficiency as in the case of a Nox 3−/− knockout combined with a TLR4−/−, can prevent the development of emphysema in TLR4−/− transgenic mice.

Human subjects with COPD have increased Nox3 expression in their lungs as compared to normal human subjects without COPD.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of identifying an individual at risk of developing emphysema/COPD, said method comprising the steps of:
a) measuring the level of Cathepsin E present in a body sample obtained from an individual suspected to be at risk of developing emphysema/COPD;
b) comparing said level of Cathepsin E in said body sample to the level of Cathepsin E present in a body sample obtained from an otherwise identical individual not at-risk of developing emphysema/COPD;
wherein, when said level of Cathepsin E is elevated in the individual suspected to be at risk compared to the level of Cathepsin E present in a body sample obtained from an otherwise identical individual not at-risk of developing emphysema/COPD, said individual is at-risk for developing emphysema/COPD.

2. The method of claim 1, wherein said individual is a mammal.

3. The method of claim 2, wherein said mammal is a human.

4. The method of claim 1, wherein said body sample is selected from the group consisting of a tissue, a cell, and a body fluid.

5. The method of claims 4, where said body fluid is urine.

6. The method of claim 1, wherein said measuring of said Cathepsin E comprises an immunoassay for assessing the level of said Cathepsin E in said sample.

7. The method of claim 6, wherein said immunoassay is selected from the group consisting of Western blot, ELISA, immunoprecipitation, immunohistochemistry, immunofluorescence, radioimmunoassay, dot blotting, and FACS.

8. The method of claim 1, wherein said measuring of said Cathepsin E comprises a nucleic acid assay for assessing the level of a nucleic acid encoding said Cathepsin E in said sample.

9. The method of claim 8, wherein said nucleic assay is selected from the group consisting of a Northern blot, Southern blot, in situ hybridization, a PCR assay, an RT-PCR assay, a probe array, and a gene chip.

10. A method of identifying an individual having emphysema/COPD, said method comprising the steps of:
a) measuring the level of Cathepsin E present in a body sample obtained from an individual suspected to have emphysema/COPD;
b) comparing said level of Cathepsin E in said body sample to the level of Cathepsin E present in a body sample obtained from an otherwise identical individual who does not have emphysema/COPD;
wherein, when said level of Cathepsin E is elevated in said individual suspected to have emphysema/COPD compared to the level of Cathepsin E present in a body sample obtained from an otherwise identical individual who does not have emphysema/COPD, said individual has emphysema/COPD.

11. The method of claim 10, wherein said individual is a mammal.

12. The method of claim 11, wherein said mammal is a human.

13. The method of claim 10, wherein said body sample is selected from the group consisting of a tissue, a cell, and a body fluid.

14. The method of claims 13, where said body fluid is urine.

15. The method of claim 10, wherein said measuring of said Cathepsin E comprises an immunoassay for assessing the level of said Cathepsin E in said sample.

16. The method of claim 15, wherein said immunoassay is selected from the group consisting of Western blot, ELISA, immunoprecipitation, immunohistochemistry, immunofluorescence, radioimmunoassay, dot blotting, and FACS.

17. The method of claim 10, wherein said measuring of said Cathepsin E comprises a nucleic acid assay for assessing the level of a nucleic acid encoding said Cathepsin E in said sample.

18. The method of claim 17, wherein said nucleic assay is selected from the group consisting of a Northern blot, Southern blot, in situ hybridization, a PCR assay, an RT-PCR assay, a probe array, and a gene chip.

19. A method of treating a mammal diagnosed with emphysema/COPD, wherein said emphysema/COPD is characterized by lung cell apoptosis, alveolar destruction, increase in lung volume, or loss of lung elasticity, said method comprising administering to said mammal a composition comprising a therapeutically effective amount of at least one Cathepsin E inhibitor wherein said composition attenuates, prevents, or halts said lung cell apoptosis, alveolar destruction, increase in lung volume, or loss of lung elasticity.

20. The method of claim 19 wherein said Cathepsin E inhibitor comprises an antibody, siRNA, a ribozyme, an antisense, an aptamer, a peptidomimetic, a small molecule, or any combination thereof.

21. The method of claim 20, wherein said antibody comprises an antibody selected from a polyclonal antibody, a monoclonal antibody, a humanized antibody, a synthetic antibody, a heavy chain antibody, a human antibody, and a biologically active fragment of an antibody.

22. The method of claim 19, wherein said mammal is a human.

23. A method of preventing a mammal at-risk of developing emphysema/COPD from developing emphysema/COPD, wherein said emphysema/COPD is characterized by lung cell apoptosis, alveolar destruction, increase in lung volume, or loss of lung elasticity, said method comprising administering to said mammal a composition comprising a therapeutically effective amount of at least one Cathepsin E inhibitor, wherein said composition prevents said mammal from developing said lung cell apoptosis, alveolar destruction, increase in lung volume, or loss of lung elasticity.

24. The method of claim 23, wherein said Cathepsin E inhibitor comprises an antibody, siRNA, a ribozyme, an antisense, an aptamer, a peptidomimetic, a small molecule, or any combination thereof.

25. The method of claim 24, wherein said antibody comprises an antibody selected from a polyclonal antibody, a monoclonal antibody, a humanized antibody, a synthetic antibody, a heavy chain antibody, a human antibody, and a biologically active fragment of an antibody.

26. The method of claim 23, wherein said mammal is a human.

27. A method of treating a disease associated with dysregulation of the TLR4 pathway in lung, wherein said dysregulation includes increased Cathepsin E expression, said method comprising administering a therapeutically effective amount of at least one Cathepsin E inhibitor to a mammal wherein said Cathepsin E inhibitor attenuates, prevents, or halts the dysregulation of said TLR4 pathway, thereby reducing said Cathepsin E expression in the lungs of said mammal.

28. The method of claim 27 wherein said Cathepsin E inhibitor comprises an antibody, siRNA, a ribozyme, an antisense, an aptamer, a peptidomimetic, a small molecule, or any combination thereof.

29. The method of claim 28, wherein said antibody comprises an antibody selected from a polyclonal antibody, a monoclonal antibody, a humanized antibody, a synthetic antibody, a heavy chain antibody, a human antibody, and a biologically active fragment of an antibody.

30. The method of claim 27, wherein said mammal is a human.

31. A method of preventing a disease associated with dysregulation of the TLR4 pathway in lung, wherein said dysregulation includes increased Cathepsin E expression, said method comprising administering a therapeutically effective amount of at least one Cathepsin E inhibitor to a mammal wherein said Cathepsin E inhibitor attenuates, prevents, or halts the dysregulation of said TLR4 pathway, thereby reducing Cathepsin E expression in the lungs of a mammal.

32. The method of claim 31 wherein said Cathepsin E inhibitor comprises an antibody, siRNA, a ribozyme, an antisense, an aptamer, a peptidomimetic, a small molecule, or any combination thereof.

33. The method of claim 32, wherein said antibody comprises an antibody selected from a polyclonal antibody, a monoclonal antibody, a humanized antibody, a synthetic antibody, a heavy chain antibody, a human antibody, and a biologically active fragment of an antibody.

34. The method of claim 31, wherein said mammal is a human.

35. A method of treating a mammal diagnosed with emphysema/COPD, wherein said emphysema/COPD is characterized by lung cell apoptosis, alveolar destruction, increase in lung volume, or loss of lung elasticity, said method comprising administering to said mammal a composition comprising a therapeutically effective amount of at least one Nox 3 inhibitor wherein said composition attenuates, prevents, or halts said lung cell apoptosis, alveolar destruction, increase in lung volume, or loss of lung elasticity.

36. The method of claim 35 wherein said Nox 3 inhibitor comprises an antibody, siRNA, a ribozyme, an antisense, an aptamer, a peptidomimetic, a small molecule, or any combination thereof.

37. The method of claim 36, wherein said antibody comprises an antibody selected from a polyclonal antibody, a monoclonal antibody, a humanized antibody, a synthetic antibody, a heavy chain antibody, a human antibody, and a biologically active fragment of an antibody.

38. The method of claim 35, wherein said mammal is a human.

39. A method of preventing a mammal at-risk of developing emphysema/COPD from developing emphysema/COPD, wherein said emphysema/COPD is characterized by lung cell apoptosis, alveolar destruction, increase in lung volume, or loss of lung elasticity, said method comprising administering to said mammal a composition comprising a therapeutically effective amount of at least one Nox 3 inhibitor, wherein said composition prevents said mammal from developing said lung cell apoptosis, alveolar destruction, increase in lung volume, or loss of lung elasticity.

40. The method of claim 39, wherein said Nox 3 inhibitor comprises an antibody, siRNA, a ribozyme, an antisense, an aptamer, a peptidomimetic, a small molecule, or any combination thereof.

41. The method of claim 40, wherein said antibody comprises an antibody selected from a polyclonal antibody, a monoclonal antibody, a humanized antibody, a synthetic antibody, a heavy chain antibody, a human antibody, and a biologically active fragment of an antibody.

42. The method of claim 39, wherein said mammal is a human.

43. A method of treating a disease associated with dysregulation of the TLR4 pathway in lung, wherein said dysregulation includes increased Nox 3 expression, said method comprising administering a therapeutically effective amount of at least one Nox 3 inhibitor to a mammal wherein said Nox 3 inhibitor attenuates, prevents, or halts the dysregulation of said TLR4 pathway, thereby reducing said Nox 3 expression in the lungs of said mammal.

44. The method of claim 43 wherein said Nox 3 inhibitor comprises an antibody, siRNA, a ribozyme, an antisense, an aptamer, a peptidomimetic, a small molecule, or any combination thereof.

45. The method of claim 44, wherein said antibody comprises an antibody selected from a polyclonal antibody, a monoclonal antibody, a humanized antibody, a synthetic antibody, a heavy chain antibody, a human antibody, and a biologically active fragment of an antibody.

46. The method of claim 43, wherein said mammal is a human.

47. A method of preventing a disease associated with dysregulation of the TLR4 pathway in lung, wherein said dysregulation includes increased Nox 3 expression, said method comprising administering a therapeutically effective amount of at least one Nox 3 inhibitor to a mammal wherein said Nox 3 inhibitor attenuates, prevents, or halts the dysregulation of said TLR4 pathway, thereby reducing Nox 3 expression in the lungs of a mammal.

48. The method of claim 47 wherein said Nox 3 inhibitor comprises an antibody, siRNA, a ribozyme, an antisense, an aptamer, a peptidomimetic, a small molecule, or any combination thereof.

49. The method of claim 48, wherein said antibody comprises an antibody selected from a polyclonal antibody, a monoclonal antibody, a humanized antibody, a synthetic antibody, a heavy chain antibody, a human antibody, and a biologically active fragment of an antibody.

50. The method of claim 47, wherein said mammal is a human.


SEQUENCE LISTING (76 sequences)

SEQ ID NO: 1 - protein, 397 aa, Mus musculus
MKPLLVLLLLLLLDLAQAQGALHRVPLRRHQSLRKKLRAQGQLSEFWRSHNLDMTRLSESCNVYSSVNEPLINYLDMEYFGTISIGTPPQNFTVIFDTGSSNLWVPSVYCTSPACKAHPVFHPSQSDTYTEVGNHFSIQYGTGSLTGIIGADQVSVEGLTVDGQQFGESVKEPGQTFVNAEFDGILGLGYPSLAAGGVTPVFDNMMAQNLVALPMFSVYLSSDPQGGSGSELTFGGYDPSHFSGSLNWIPVTKQAYWQIALDGIQVGDTVMFCSEGCQAIVDTGTSLITGPPDKIKQLQEAIGATPIDGEYAVDCATLDTMPNVTFLINEVSYTLNPTDYILPDLVEGMQFCGSGFQGLDIPPPAGPLWILGDVFIRQFYSVFDRGNNQVGLAPAVP

SEQ ID NO: 2 - DNA, 1804 bp, Mus musculus
gagtgcccctgccctgtgatcaagaggaaagaagggtggaagggagatcggagcagagtgagagagaagctaccccttggaccacaatgaagcccctccttgtgctgctgctgctgctgctcctggatctggctcaggcccaaggtgctctgcacagagtgcccctcagaagacatcagtcccttcggaagaaactacgggcccaaggacagctctcagaattctggaggtctcataacttggacatgacccgactcagcgagtcctgtaatgtgtattcgagtgtcaatgaacccctcatcaactacctggatatggaatactttggcaccatctccatcggcaccccgccgcagaacttcactgtcatctttgacaccggttcatccaacctctgggtcccttctgtgtactgcaccagcccagcatgcaaggcacacccagtattccatccatcgcagtccgacacgtacacggaggtagggaatcatttctccatccagtatggtaccgggagcctgacaggaatcattggagctgatcaagtctctgtggaagggttgactgtggatggccagcagtttggagaaagtgtcaaggagccaggccagacctttgtgaatgcagagtttgatgggattctgggtctgggatacccctcattggctgctggaggagtgaccccagtgtttgacaacatgatggcccagaaccttgtggctctgcctatgttttctgtctacttgagcagtgaccctcaaggtggctcaggcagtgagctgactttcggaggctatgacccctctcatttctctgggagcctcaactggatcccagtcaccaagcaagcctattggcagattgccctggatggaatccaggtgggagacactgtgatgttctgctccgaaggctgccaggccatagtggacacagggacctctctcatcactggcccccccgacaagatcaaacagcttcaagaggccattggggccacacccattgatggagaatatgcagtggattgtgccactctcgacacgatgccaaacgttaccttcctcatcaacgaggtttcatataccctcaacccaactgactacatcctgccggacttggtggaaggaatgcagttctgtggcagtggctttcaaggacttgacattccacctccagctgggcccctctggatcctgggggatgtcttcatccgacagttctactcagtctttgaccgtggaaataaccaagtgggattggcccccgcagttccctaaagagggatgtatgcctacatatggatgcctgatacccatttaacctgttagatacctttgtaactatcaaagccgtcatttcccatggggtgtagccaccccagagtattcagaccaatcaaagcataagagtgcaccccactcactgcaaacacacacacacacacaccacctctaccatcaccacgatgaaagaagttctgtctatagtcttcactgcttattgttgactttctattatggaaatctctaaacatgtacacagtagacatgatggcaagataaatacccacacacctctgcctcaggtcacaacccatccatgtgtggcccagactctctatcttccatccctctggttccacgcctctagatttggaagcagattctaagcaccaggtcattttatctaatgtctaacatccttacaaatcagaatttaaatgtctcaccctctcataaatgtggacctgtttttacagttggtttatttgtatcaggattaaaaccagatccataactgg

SEQ ID NO: 3 - DNA, 20 bp, Artificial
  Chemically synthesized
acacgcatacccacacatac

SEQ ID NO: 4 - DNA, 20 bp, Artificial
  Chemically synthesized
tactggagtcactcctccca

SEQ ID NO: 5 - DNA, 25 bp, Artificial
  Chemically synthesized
gtcgctaaagaagaaagggaaacac

SEQ ID NO: 6 - DNA, 25 bp, Artificial
  Chemically synthesized
ttccaagggcatcggtaaacatctg

SEQ ID NO: 7 - DNA, 24 bp, Artificial
  Chemically synthesized
tgccacgaccaagtgacagcaatg

SEQ ID NO: 8 - DNA, 18 bp, Artificial
  Chemically synthesized
agagacggaaatccatcg

SEQ ID NO: 9 - DNA, 20 bp, Artificial
  Chemically synthesized
cgatgcaacgagtgatgagg

SEQ ID NO: 10 - DNA, 20 bp, Artificial
  Chemically synthesized
cgcataaccagtgaaacagc

SEQ ID NO: 11 - DNA, 25 bp, Artificial
  Chemically synthesized
gtcgctaaagaagaaagggaaacac

SEQ ID NO: 12 - DNA, 25 bp, Artificial
  Chemically synthesized
ttccaagggcatcggtaaacatctg

SEQ ID NO: 13 - DNA, 20 bp, Artificial
  Chemically synthesized
tcccctctgtgtactgcact

SEQ ID NO: 14 - DNA, 20 bp, Artificial
  Chemically synthesized
tactggagtcactcctccca

SEQ ID NO: 15 - DNA, 20 bp, Artificial
  Chemically synthesized
acacgcatacccacacatac

SEQ ID NO: 16 - DNA, 20 bp, Artificial
  Chemically synthesized
tactggagtcactcctccca

SEQ ID NO: 17 - DNA, 20 bp, Artificial
  Chemically synthesized
tcccctctgtgtactgcact

SEQ ID NO: 18 - DNA, 20 bp, Artificial
  Chemically synthesized
tactggagtcactcctccca

SEQ ID NO: 19 - DNA, 19 bp, Artificial
  Chemically synthesized
gctagcagtggatgggccc

SEQ ID NO: 20 - DNA, 20 bp, Artificial
  Chemically synthesized
atcagccactgagtggagct

SEQ ID NO: 21 - RNA, 19 nt, Artificial
  Chemically synthesized
gcgcgcuuuguaggauucg

SEQ ID NO: 22 - RNA, 19 nt, Artificial
  Chemically synthesized
cgaauccuacaaagcgcgc

SEQ ID NO: 23 - DNA, 20 bp, Artificial
  Chemically synthesized
tgtcatctcgctctggtacg

SEQ ID NO: 24 - DNA, 20 bp, Artificial
  Chemically synthesized
taacgcgagtgagaatgtgc

SEQ ID NO: 25 - DNA, 20 bp, Artificial
  Chemically synthesized
gcctacgggatagctgtcaa

SEQ ID NO: 26 - DNA, 20 bp, Artificial
  Chemically synthesized
ggactgcagatgggtgactc

SEQ ID NO: 27 - DNA, 21 bp, Artificial
  Chemically synthesized
gtgggccgctctaggcaccaa

SEQ ID NO: 28 - DNA, 24 bp, Artificial
  Chemically synthesized
ctctttgatgtcacgcacgatttc

SEQ ID NO: 29 - DNA, 21 bp, Artificial
  Chemically synthesized
gcaggacttccaaaagaacga

SEQ ID NO: 30 - DNA, 19 bp, Artificial
  Chemically synthesized
gacgaatgcctgccacaag

SEQ ID NO: 31 - DNA, 22 bp, Artificial
  Chemically synthesized
gagaccctaccctggactacca

SEQ ID NO: 32 - DNA, 20 bp, Artificial
  Chemically synthesized
cccagatgagacgccgtact

SEQ ID NO: 33 - DNA, 21 bp, Artificial
  Chemically synthesized
cccctccattcattgcaagat

SEQ ID NO: 34 - DNA, 20 bp, Artificial
  Chemically synthesized
caaaggacgtgccgttcttc

SEQ ID NO: 35 - DNA, 20 bp, Artificial
  Chemically synthesized
gattgtgcccaagccttcaa

SEQ ID NO: 36 - DNA, 21 bp, Artificial
  Chemically synthesized
ctgtcttcttccatgatgccc

SEQ ID NO: 37 - DNA, 21 bp, Artificial
  Chemically synthesized
catttcgcctctctgctgatg

SEQ ID NO: 38 - DNA, 23 bp, Artificial
  Chemically synthesized
ttgatggtggactgctaggtttt

SEQ ID NO: 39 - DNA, 19 bp, Artificial
  Chemically synthesized
ccctggaactcacacgaca

SEQ ID NO: 40 - DNA, 20 bp, Artificial
  Chemically synthesized
ggaaactcacacgccagaag

SEQ ID NO: 41 - DNA, 21 bp, Artificial
  Chemically synthesized
tccctggaattggcaacaaag

SEQ ID NO: 42 - DNA, 24 bp, Artificial
  Chemically synthesized
ggaatttgttggcatgactctcac

SEQ ID NO: 43 - DNA, 20 bp, Artificial
  Chemically synthesized
aaggctgatttggcaaccat

SEQ ID NO: 44 - DNA, 19 bp, Artificial
  Chemically synthesized
agcgcttcctccgaacatt

SEQ ID NO: 45 - DNA, 20 bp, Artificial
  Chemically synthesized
ccttccataagctggtggca

SEQ ID NO: 46 - DNA, 22 bp, Artificial
  Chemically synthesized
ggcctgttggcttcttctgtag

SEQ ID NO: 47 - DNA, 24 bp, Artificial
  Chemically synthesized
cttatcacagccacaagcattgaa

SEQ ID NO: 48 - DNA, 22 bp, Artificial
  Chemically synthesized
cacccattcacactgacctctg

SEQ ID NO: 49 - DNA, 20 bp, Artificial
  Chemically synthesized
ctcgttgcctacgggatagc

SEQ ID NO: 50 - DNA, 19 bp, Artificial
  Chemically synthesized
ccttcagcatccttggcct

SEQ ID NO: 51 - DNA, 26 bp, Artificial
  Chemically synthesized
acaaccaagggccagaatactactac

SEQ ID NO: 52 - DNA, 20 bp, Artificial
  Chemically synthesized
ggatgaggctgcagttgagg

SEQ ID NO: 53 - DNA, 24 bp, Artificial
  Chemically synthesized
tctgatcatggcactgttcttctc

SEQ ID NO: 54 - DNA, 21 bp, Artificial
  Chemically synthesized
gggattcaagcttcctggtgt

SEQ ID NO: 55 - DNA, 23 bp, Artificial
  Chemically synthesized
ctgaactcaactgtgaaatgcca

SEQ ID NO: 56 - DNA, 20 bp, Artificial
  Chemically synthesized
aaaggtttggaagcagccct

SEQ ID NO: 57 - DNA, 19 bp, Artificial
  Chemically synthesized
agttcccaaatggcctccc

SEQ ID NO: 58 - DNA, 20 bp, Artificial
  Chemically synthesized
gctacgacgtgggctacagg

SEQ ID NO: 59 - DNA, 27 bp, Artificial
  Chemically synthesized
agaaaacaatctgaaacttccagagat

SEQ ID NO: 60 - DNA, 26 bp, Artificial
  Chemically synthesized
gaagaccagaggaaattttcaatagg

SEQ ID NO: 61 - DNA, 65 bp, Artificial
  Chemically synthesized
gatcccatacacattacaggactcgttgatatccgcgagtcctgtaatgtgtatttttttccaac

SEQ ID NO: 62 - DNA, 65 bp, Artificial
  Chemically synthesized
tcgagttggaaaaaaatacacattacaggactcgcggatatcaacgagtcctgtaatgtgtatgg

SEQ ID NO: 63 - DNA, 20 bp, Artificial
  Chemically synthesized
gcacctttgatatgggcaca

SEQ ID NO: 64 - DNA, 64 bp, Artificial
  Chemically synthesized
gatcccgcacctttgatatgggcacaactcgagttgtgcccatatcaaaggtgctttttccaac

SEQ ID NO: 65 - DNA, 64 bp, Artificial
  Chemically synthesized
tcgagttggaaaaagcacctttgatatgggcacaactcgagttgtgcccatatcaaaggtgcgg

SEQ ID NO: 66 - DNA, 13257 bp, Mus musculus
ctattacacatagtgtttactctgggcatggctattgactccagaccttatcattgggtcctcagactgggcagggcaggtctgggcaccaggaagagccagtgcccctgccctgtgatcaagaggagagaagggtggaagggagatcggagcagagtgagagagaagctaccccttggaccacaatgaagcccctccttgtgctgctgctgctgctgctcctggatctggctcaggcccaaggtgctctgcacaggtaaggccctgtctccagcactaagttcaattgaggcatcccccaaccccaacccagggctgccccgggggaggaaagctaatgaggaaatgaaaagaagaaaccgggatctctcaggaatccctagagaagattccccaggcctctccttccagtgtaggtgcctgcctcagctcaagggactccccctctgttgttcaaaggtgtgctccatagatgtgatttgtgactccaagccttcaaatactttcccaagggggcctgagcactgctatccacagggtccttccatccctttaagtcagccctttatgatcagactatagactgtgtctgacctcccattccctagaaaatctcaaaaggccagtgcccaggagggaaagccatttctacccaaattctgagtctgcaagacaaagccattgctgcttctggtcttgaaggttccctcctgggtcttgctgtagtcaggctctgaacaggtatagaggcaatgggagcctttatttataaatgcccctttatttcctgatctttctccagagtgcccctcagaagacatcagtcccttcggaagaaactacgggcccaaggacagctctcagaattctggaggtctcataacttggacatgacccgactcagcgagtcctgtaatgtgtattcgagtgtcaatgaacccctcatcaactacctggatgtaagatttctcaaattgaagcctgaggacactgggtgtagggaggaaggctggggccttcagcaggggccagtgctgcattggtggcattatgatcaggcatggatttgtttgcttggaggattcagacagtcccccagtctctatctactctgtaaagcaggcttctaaatgacccagtatgcagagctttcttcaagaggaaagagataagtatgatatgtgttccaaaaagaaagacccctactgtgtgcccagctgggcattttactagctattcctctgggcaaagaacttggtttgccacacctccaggtccctgatgacatcgtgaggatcaggataatgagagaaggatggacccaagtttcctgggctatcctcagtttcctccctggtggtccttgctctgttccttccatcctttgcctccagccttgcctcttctgggctctgtagattgtagcttgtggtacttcatgatttagtgcccttcccctccactccacccacacactctctctctctctcttccctacagatggaatactttggcaccatctccatcggcaccccgccgcagaacttcactgtcatctttgacaccggttcatccaacctctgggtcccttctgtgtactgcaccagcccagcatgcagtaagtgaacttggcagtggcgggagctcactaggctaagatggtaccttgtggcatagagagactcaggttctctccacttatcttccctgccatactggcttccccttcctatatctgtttgttgtgcctgagccaagcacagacaagaaaggcaactaccagttaagataacaaccttcatgttcattagaattgatggagagaaaattagccattaggacgtggtgactcacttcctttggaacacccaaaggaacatgggggaaagttccttcggtctgtacctgaaccgatagctagattgaattctaaggaaaactgtgctcctgctccagggaggccctgatgcactggctcccttggaggtgtggctcgtgggacacagtggtgtaggtgtgtccatttgaccagaggtaatgtaggggctacatagagattagaaacagacaccctaaacagagaaagcttgttctgtgtgggacacatctcaaggacagaagctctttgaaactctgacaacagacattgtcaaggtcaggtgaggaggtaggagtgatttcttagagtagtaattgaaactcggtgtgggtcgggcccacactgatggctgaggctgctgctggcgcctggtggacggtgcttatcttgtgtctatgaagtcactgtgactaaccaggtttgtccagggacaaatatttggcccctggctagaacacacctgtggctagggaacacagccatggtatgcctgtctaggaagccccatcttcaacctagtgtggggagcagaggggagccactccacccccctgggatggggtggaagactcaagcagcattcttgctttccagaggcacacccagtattccatccatcgcagtccgacacatacacggaggtagggaatcatttctccatccagtatggtaccgggagcctgacaggaatcattggagctgatcaagtctctgtgagtacaatgcccacatcttgccccagaactaggcctggggatagcaggatatgattcaaagcctcaggggcacagtttcagctctgccttgggacgagaagacaaggttatttctagcacaagggaacagctcgttctacctgagatagcccggtggccatctttgcaggatgctgaacctagagagcagtgtggggcttccttcagggaacaagggtcagtcagggcatgatgccttgtgtcagggctggatcaggaaaccctgggaactgaggggataagaacaggctgccatcttgaaagaaaaaagatgttgaagcccgtagacaggctggggctccattagtggagtgggaaggcaggaggcaggaatccaagacagggaagaagccaggtatcaaagccccaagttccttgaacttctcatggccagaggtcccgaatgtccctgatttgtaggaatcagagagtcaggagggccctggtcctgcctggatactttatgcaagtgtatagtcccagggcttggtagaaatagagaatgggccacccggatgggtgagtattattctcgatctagcctgatccaaaggccagcaaagcttagtcttcatcagcatctcttccaaactttgaacatgggatatatgattaactgaaaaagggctgctaacagaaatctctcttttcttctctgtccccctcctcacattaaacctggtgcatatctttctgggcccttatgacgtgtgtggggtcggagtactgtagtgagctggtcatcaaatagtataaagcaaagcaaatatgtcaatggccttaattattctctttagggtagtaggtctccttaaaatatgtctaagtgggttggatagatggctcagtggttaagagcacagactgctcttccaaaagaccccggttcaatccccagtacccaatgtcagctcgtatctgtctgtagctccagtcccagcagagccaggactcagacacaagtgcaggaaaacaccaatgcaaagaaagagaagccaatacaagaaaaagaaatcactaaagacaatttaaaaaaacaatttaagtaaagccagctatggtggcacacacctttaatcccagaacttagaaagctgcagccaatggatctctgagttcgaggccagcctagcctacatagagagttccaggccagctaaggactacatagtgaaaccctgtctccaaacttgaaaaaagtttcagtaatgtcttcttatcataaaaatgctttttatgtaaaattatataaatgaagcagagctatcagttgaaattttacatgcatatgtaaatacacattatgtgtatatgttacagtataaataaccacttggaaggctgcatgtcaacctgatcattgtagaaagtctggagagcagggaggagactgagatctacaggaggtgggctctggcatacagcttctcgcctctctgctagttgccctgaacagagcagagagcagccaagcccatagcaacttcagctgcaatggcctctccttcagcacttctggctctggggcaggtgcccatttgcctctgtggcagtgttagcttctactgtgaagcattccttcactgaatatagaggacatcatggtgccagtccatccctacaggctgcagctggcatctttcccacttggcacgtagcttccctctgctgctgcttctgtagacctcaggctgcaaccctaggttagcaaccacatcctaagtgcactcccagctggcatctgcaatggtataaagccaagtacctaacacatatatagcatataagcacacagattagtacagttcatgcagcctgaaatgtaaagatcagaaaatcagtgatctaagccgcggtttggagtaacaaacactaagaaacaagctgttaataaacaggcgcaagccaaacccaagggaaagaaatcacatagagaaggacagggcatcggtacaataggaaatccacactataaacatacagccaggcgtggtggcgcacacctttaattccagcacttgggaggtagaggcagaggcaggcagatttctgagttcgaggccagcctggtctacaaagtgagttccaggacagccaggactacaaagagaaaccctgtctcaaaaaacatacaaacaaaaaaacaaaacaaaaaaatcaactacgtcacagattaactgtttgcaaagagcaaccaaattggtaaccctctggcataactaatccagaaaaatgaaggaaatacaatgaccttgtcaatctcaaaacaagatgtcaaaaaatgcttgatattgatacactacaataattttaaatagatgactatcaaaactgatacaataaaatagaaatgattaataatctggtatctattttaagtggtgattctgctattaaaacaacaacaaaacaaaacaaaaaacccacttcccacaaggaagtgacagatccatcttccagagaattgatagaattccttcataacttcaccagaacaactatgataacaaaatgtgaaaagattcttatacacactgagacttgctagacatagtggcacaggcctttaatccctgtactcagggggcagaggcaggcagacctctatgaatgtcaggccagtctggtccacatagagaattttaggactgccaagactatgtagtgagaccatgtctcaaccagtcgcccagtgcagtgagcagagtgcttgtctagtgtacgcaaagcccagaattgcatccccaggaccacataaactggacaaggtgatacatatgtataatcccagcacccaggaggcagaggcaagagggtcagaagttgaagatttcctccattgcatagtatgctcagtgctagcctgggctatatgagatcctgtaggaatatgtaatgtgttcaccagaggaagtgccagtgcaccattattgataatcaaatattggaaacagcccagctatccacccatagtataaggcagtgtttctcaacctgtgggtagcaacctctttggatcaatttgtaacaatgaaaatataagccacacacacacacacacacacacacacacacacacacacattatatatatacatatataatgtaatatatgtgtgtgtatttacattatgatccacaatagtagcaaaattacagttacaaagtagcagcgaaaatgtcatggcgggtggtcatcacaacatgagaaactgtattaaaggttgccccagtagtaggaaggctgatgaccccaaaaataggcaaaaccaatgttgcttgagagatgagtcactcttgagggatacaaagactggcagggtatatgaaaggcctgctggggcttcggattgtcctgtcttatttcagaagggttacataagaatgtggactgttattaaaaaatagacactcatgttttctgcattacattatattttgatttttaaagtcacatgaagaaagtccaagagtgggattatagtgaaaatctgtgtgcctatttccccaggaggctgagaaaggaagatctagcttcacccgggaatctgagaccctccttcaactaacctttgtgtaattctcccttcaggtggaagggttgactgtggatggccagcagtttggagaaagtgtcaaggagccaggccagacctttgtgaatgcagagtttgatgggattctgggtctgggatacccctcattggctgctggaggagtgaccccagtgtttgacaacatgatggcccagaaccttgtggctctgcctatgttttctgtctacttgagcaggtgaggccagtcaagtcactgaggtccaatcagtgaatgactacaggcagatggcctggtacataagacttcaaggttctgaggcccatgaatctctcgaatctcttccttggcctatgatgtatatgatcttgagaaaaaaaaaacaaaaacaaaaacaaaaacaaaaaaaccctgaagcgatttggatttcaaggaaatggggtttgactcataaaagagttgagttgagcactttacaatctagcacggtgagttaaagttatagaccgttgcgattggctagtgatttatttggtcattaaattctaaagtccttttatatgctaagtgtgacagtagacaccaaaacacaaggaaacaagagtagtctccgtggttgggagagaaggaaaagattaaccagcaagatgatactaaaagtaacttctacattagggcgaggaaaggaaactgtagtatagccatgtagtagaatatcacttggtctctggaaggaaaaccagtgagggatgcagtgtgcagtagaggacttacctagctgacacaaggctccgggttcactctccaatgcctcacagacatgtgctagaacatggattatgctaaatgaactaagccagtagcccaaaggacagatacaaattatactttatgcgaggtacctagaactgaattcataggcgaaaagtgcagtggtggttgcgacggggtagagggataaataggaagggggttctcgcttaatgagtctcagttctacactgtgaaaatgttctggagaacagcttcccaggaatgaaattctgaagtgcacactcaaaaaatggttaagcttgttctatcttatggctgtactgccacgagttttaaagatgcacacactacagttaaaaagagcatggagctcaccgctctagcctggggtgctgaggaagtcactttcctacctacacagaaagatttacctttcccactctggcctggcctggcctgagctcgccacattccagtgtaagtcccctttccctgtccccctcccacatcagaagttcctggagtgagtggctgcagctgggaccaggcctgggtagggttttgattgagcccctgctgtcagaactcctgtttcctctgcagaggctcctgaactacctgctaaagaagtgtgcagctttggtcagtagcaaggccagcatagcctggaactaagtctgcatgtttgtctcccagggcctagggttcctcaggcaaatgtgaggctgctgtgagaattcaacaagatcgggtggagtgagaattttggtttcttcaaagacagaaatgtcagcggaatctgttttcattttaatccctggtgtggcatatggggctgctttagattgtccacagcagccaactatgatttacccaatgcatgctctagagggggcatgattttgccagctgcagataggttctgcaggtatatgatgtttgaaagtctggagactcttgagagggtatagaagctgctgctggtggtggttgctgctgatgcggtttgtcaagtagtcacgcacaaagggtcacaagaaggaaaaattagatagcctgattgctaagatcgaacttgtcccaaggaactctatgcccctaatcagcaggaagtaatctaacaatatcattgacccctttcctctcaaccttctttctctctacctagttcttagggtgggggcaaatggaagggtgaaataggggtgaagaagagtggcaggaaaaagaacctataaagtagccaaaagttggcttactgtcatgtggttagattgctagaacccggcaagcaagtgttctaccccttagctatgtcctcagtccccaataaacctgaaattctctactgacatatacttccttccctactgctactctatcctgcagcaccccaggcttagactctttcctcctggaaatgccctcctttccagaatcctcctaaatcccagcataaaacttgagaaataactaccaaggaaaattagccgatagtaaagccccagtaagacatgttgctttcaaatgatcttttctgaagaagtggatacggtagaaaccccagacaccacagcatcaatagcagggaagatactgtgagaaagagtattttgggggcatcctcagttgcacagtgtttgtacccaacacccaagatagtcctgaccttagatctagagttcagatactccctacctcctgaactttttatctaagaagcaggggatatgcacagggtgttagacagagttgttcctatctttgatccccaaagacgcaggctacctaggtagtttctgcctactttgccaccagatgtgagtgcccggacatgcaacaaaggggccctgaactcctgtctgtgtttccagaggccatttcttgacaacctgcctttctctctgcatagtgaccctcaaggtggctcaggcagtgagctgactttcggaggctatgacccctctcatttctctgggagcctcaactggatcccagtcaccaagcaagcctattggcagattgccctggatgggtgagtatcctctagatagtagctatggttgaatggaagagtggccacaactcaaactggattaactgggtgatgctgcaggacactagaacagatgctgggagcctcacatctggactggagatgtgaaaacacacaggaaggacaagaggggcagcttccttctgtgtacatttagctggcctccctctgactcacacaaccagctatcctgtcttcaagcagccttaattgatacacaaggcaagggctgagcaccagggaaaagacactggctagctgaactcaaagcctgtatggaatatgaggtgtggtcccaaatcaccaccacacaaaatggatgtggtcagtgttctgaaagccaccggtatggccagccaacagcacaagatgtctctgtctgaagactgctcatgttttgtggaggcaatgactctggaactgagccttgaaagccatgcctgggcatgaagcaaatggggaagcagcattgcgtacctgatagatatatggccagccccgtgctagttaaggggacagtttaaggagaacaatctagtttctgttcctcagatgatctggataaaaagtgaaagttccaactctttgtagctcctgacgcttttccgactgagatttacctatttatgctcatgagatcgttttaaggaatctcggtccgtttgcttggttttattttttgaggcaggatcttcccatgtagccccagctgtcctagaacgctcaggatcctgtcagcctcctgcatgctggagttacaggtgtccccaccatgcccagctctccatctgcatttctgaataaaaacagagcacagtagaaaattcaagaatgtatttcagtgtgtgtgtgttcatgtgctcgtgcatacttgatcatgaattaaatgtattcctgccctaaatctcagtcaaaaaagcaagttgagtcaagtataatggctcatgcctataattccagcatccatgaggactgctatgggtttgagattagcctggacaggatgaagatgtcttaaaaagaagcaagttggccaggcatagtggtgcatgcctttaatcccagcacctgggagacaaaggcaggtggatctctgaattcaaggccagtctatatagtgagttctagaaccaccaaagcgatgcagagagaccctatctcaaacaaacaaatatgctagttgaaataagctactactcctgtggtagaagagaccaaagagaacaccattattgtgacctaatatagtaaagggccccagagagagagagagagagagagagagagagagagagagagagagagagagagagagagcaggaatggccaaggatcatgtgttaaccatgggaccatgggactctttgtcctcagaatccaggtgggagacactgtgatgttctgctccgaaggctgccaggccatagtggacacagggacctctctcatcactggcccccccgacaagatcaaacagcttcaagaggccattggggccacacccattgatggagaagtgagtgtctaccgagggtggaggatggtagaggagcaggagatcctgtccttgtctggggagagagtgggggtctgagttgatccttctctgttttagtatgcagtggattgtgccactctcgacacgatgccaaacgttaccttcctcatcaacgaggtttcatataccctcaacccaactgactacatcctgccggtaagggctgtttccttattctgtgagtcacagtaccctccccacgtgcacctgccacttcccccttcaaagtcatctacatgaggctaccactcttccaaggctcctcccagctagtccaggggatgcctatgtggcattctctctgaaagagatgaagctgcctgcggtagaaactcctggacttctagtttccagttctgctatgaagcagcagctactcaggtacaagacacttaagggggatttctgatattgccatgaggaatttatcaacaaacacattaatcctccccactaccggtgtgcaactggagatgttattatcctattatacatgggaggaagctaaggtcctcagagttaaataaatgcttcagatctgactgctggggttggagagatggctcagcagttaagagcacttggatgctccaggggaccacaattgagttctgcacctgcatgtatggctcacaaaccatatgtaactccagctccaggaaatcaaatgccctcttctgatctctgtgggaccccctcatgcatgtgcacagatattgcacacatacacataaacgttttttttaagatttatgaatttattttatttatgtaagcacactgtagctgtcttcagacacaccagaagagggcatcatgtcttactacatggttgtgaactaccatgtggttgctgggaattgaactcaggacctctggaagagcagtcagtgctcttaacccctgagccatctctacagctccccacacaaaaatttttaaagagtgactttaaaatagataatgagctgcttaaggaggggccaccattcaaattacctttcttcaaggtaaaattcgtcttttattgaaatgattcccaagttctgagggtgaggtggaggtgaaagtctggacattttggaaacctcaggtcatttcccatcccccagggcctaggttttgatgttgtgctcctttggcaacacactttaatgtgttgatctttaagcacagtcccctataacatcaggatctgtgtcagcatcaagatgccaaggtagaaggtgaccttcaccttatccttcccaaagccacccgatgtaaacagacatccaaacaaacacaagcacttgctccaaggcatagtttcatggtaaagcactcaagctctagggacagtctggaacagcctctgcagacaccatgttgagcacataccactgttgaaactgagccctgctctcactctcagtctctttgccatgctactttggaccctaggacttaagttactaagtcttagtagtcagaatgcagttctgcagcagaggctctcaaggacatgacatcaccctccagctgtgccccctctgggattctgggggatgcccagctcctttactctcctacttttatccaacaagaggctctgctggtaaccacaccccggccctacctgccaggttttaatcagggatctcaacccataaaggatgctgggatagctttaaattgtatcccagtcctctcacaaggctgtggctttcccacctcttcatcagcaactttgagtgtcagatgttgggaaggccacacctgctgatagaactcaaatctttgctaaactgaaaaataagactgaaagacaggagtggcggtgaatgtctaccatcccagtggagcagaggcaggaaagggagttctaggcaacagaggttacagaatgtctcaaaaaaaaaaaaaaaaaagattgggggagtttacagtctcttcatagaacaatggatttgtgcttgccttgtgtctcagagagcccactcaaatgcttcttccccagatgtgaagaaggaagtggtagtagctagccatttatagaacagatattaagtcaagctccgcttaaaactaaaccagattgggtttaaaccaatgcgctctaacgccaaaccttctgcagtaaggttctgtttccacgtttccagttatgaactgatggtagagtatggccgtaaaaacatacagttctcagaattgtagaacacccataatgaagggagcaagcataccctgtactgagcaagctggaataggagcaggaaatgagagaaagccctccaaccacaaccgtggcactcagcaaccatccaaggccattgcacggccagcaccaccaacagtacagctcccaggactacatccattgtcccctgtctgtttcttttgtgccctaggacttggtggaaggaatgcagttctgtggcagtggctttcaaggacttgacattccacctccagctgggcccctctggatcctgggggatgtcttcatccgacagttctactcagtctttgaccgtggaaataaccaagtgggattggcccccgcagttccctaaagagggatgtatgcctacatatggatgcctgatacccatttaacctgttagatacctttgtaactatcaaagccgtcatttcccatggggtgtagccaccccagagtattcagaccaatcaa

SEQ ID NO: 67 - DNA, 156 bp, Mus musculus
agtgcccctgccctgtgatcaagaggagagaagggtggaagggagatcggagcagagtgagagagaagctaccccttggaccacaatgaagcccctccttgtgctgctgctgctgctgctcctggatctggctcaggcccaaggtgctctgcacag

SEQ ID NO: 68 - DNA, 159 bp, Mus musculus
agagtgcccctcagaagacatcagtcccttcggaagaaactacgggcccaaggacagctctcagaattctggaggtctcataacttggacatgacccgactcagcgagtcctgtaatgtgtattcgagtgtcaatgaacccctcatcaactacctggat

SEQ ID NO: 69 - DNA, 118 bp, Mus musculus
atggaatactttggcaccatctccatcggcaccccgccgcagaacttcactgtcatctttgacaccggttcatccaacctctgggtcccttctgtgtactgcaccagcccagcatgca

SEQ ID NO: 70 - DNA, 119 bp, Mus musculus
aggcacacccagtattccatccatcgcagtccgacacatacacggaggtagggaatcatttctccatccagtatggtaccgggagcctgacaggaatcattggagctgatcaagtctct

SEQ ID NO: 71 - DNA, 200 bp, Mus musculus
gtggaagggttgactgtggatggccagcagtttggagaaagtgtcaaggagccaggccagacctttgtgaatgcagagtttgatgggattctgggtctgggatacccctcattggctgctggaggagtgaccccagtgtttgacaacatgatggcccagaaccttgtggctctgcctatgttttctgtctacttgagcag

SEQ ID NO: 72 - DNA, 123 bp, Mus musculus
tgaccctcaaggtggctcaggcagtgagctgactttcggaggctatgacccctctcatttctctgggagcctcaactggatcccagtcaccaagcaagcctattggcagattgccctggatgg

SEQ ID NO: 73 - DNA, 142 bp, Mus musculus
aatccaggtgggagacactgtgatgttctgctccgaaggctgccaggccatagtggacacagggacctctctcatcactggcccccccgacaagatcaaacagcttcaagaggccattggggccacacccattgatggagaa

SEQ ID NO: 74 - DNA, 99 bp, Mus musculus
tatgcagtggattgtgccactctcgacacgatgccaaacgttaccttcctcatcaacgaggtttcatataccctcaacccaactgactacatcctgccg

SEQ ID NO: 75 - DNA, 187 bp, Mus musculus
gacttggtggaaggaatgcagttctgtggcagtggctttcaaggacttgacattccacctccagctgggcccctctggatcctgggggatgtcttcatccgacagttctactcagtctttgaccgtggaaataaccaagtgggattggcccccgcagttccctaaagagggatgtatgcctacatat

SEQ ID NO: 76 - protein, 568 aa, Homo sapiens
MMGCWILNEGLSTILVLSWLGINFYLFIDTFYWYEEEESFHYTRVILGSTLAWARASALCLNFNCMLILIPVSRNLISFIRGTSICCRGPWRRQLDKNLRFHKLVAYGIAVNATIHIVAHFFNLERYHWSQSEEAQGLLAALSKLGNTPNESYLNPVRTFPTNTTTELLRTIAGVTGLVISLALVLIMTSSTEFIRQASYELFWYTHHVFIVFFLSLAIHGTGRIVRGQTQDSLSLHNITFCRDRYAEWQTVAQCPVPQFSGKEPSAWKWILGPVVLYACERIIRFWRFQQEVVITKVVSHPSGVLELHMKKRGFKMAPGQYILVQCPAISSLEWHPFTLTSAPQEDFFSVHIRAAGDWTAALLEAFGAEGQALQEPWSLPRLAVDGPFGTALTDVFHYPVCVCVAAGIGVTPFAALLKSIWYKCSEAQTPLKLSKVYFYWICRDARAFEWFADLLLSLETRMSEQGKTHFLSYHIFLTGWDENQALHIALHWDENTDVITGLKQKTFYGRPNWNNEFKQIAYNHPSSSIGVFFCGPKALSRTLQKMCHLYSSADPRGVHFYYNKESF